SEQUENCE LISTING (231 sequences)

SEQ ID NO: 1 - DNA, 11 bp, Artificial
  Synthetic K10
acagctcagaa

SEQ ID NO: 2 - DNA, 11 bp, Artificial
  Synthetic K11
cttccgcaggt

SEQ ID NO: 3 - DNA, 9 bp, Artificial
  Synthetic K13
acgctgagt

SEQ ID NO: 4 - DNA, 10 bp, Artificial
  Synthetic K15
attggtggnc

SEQ ID NO: 5 - DNA, 9 bp, Artificial
  Synthetic K17
tcccctgca

SEQ ID NO: 6 - DNA, 9 bp, Artificial
  Synthetic K18
acgcgtcct

SEQ ID NO: 7 - DNA, 11 bp, Artificial
  Synthetic K1
atgtgcgccaa

SEQ ID NO: 8 - DNA, 11 bp, Artificial
  Synthetic K20
gtgggtagggt

SEQ ID NO: 9 - DNA, 10 bp, Artificial
  Synthetic K24
ctagccgcgt

SEQ ID NO: 10 - DNA, 10 bp, Artificial
  Synthetic K25
aatctgtccg

SEQ ID NO: 11 - DNA, 10 bp, Artificial
  Synthetic K27
gtacggtgga

SEQ ID NO: 12 - DNA, 10 bp, Artificial
  Synthetic K28
ttgcgcttct

SEQ ID NO: 13 - DNA, 10 bp, Artificial
  Synthetic K29
gtaagtctga

SEQ ID NO: 14 - DNA, 10 bp, Artificial
  Synthetic K30
attgggcctg

SEQ ID NO: 15 - DNA, 11 bp, Artificial
  Synthetic K8
gatagggggga

SEQ ID NO: 16 - DNA, 10 bp, Artificial
  Synthetic K2
gatgaccgcg

SEQ ID NO: 17 - DNA, 10 bp, Artificial
  Synthetic K34
ataaggacat

SEQ ID NO: 18 - DNA, 8 bp, Artificial
  Synthetic K35
gtaaagcg

SEQ ID NO: 19 - DNA, 12 bp, Artificial
  Synthetic K14
gtaacgcgcgga

SEQ ID NO: 20 - DNA, 10 bp, Artificial
  Synthetic K16
gtaacgccca

SEQ ID NO: 21 - DNA, 6 bp, Artificial
  Synthetic K3
atggat

SEQ ID NO: 22 - DNA, 11 bp, Artificial
  Synthetic K4
ttttgagtgat

SEQ ID NO: 23 - DNA, 11 bp, Artificial
  Synthetic K5
actcccacgct

SEQ ID NO: 24 - DNA, 10 bp, Artificial
  Synthetic K6
tgtaattcga

SEQ ID NO: 25 - DNA, 10 bp, Artificial
  Synthetic K9
ggacaacggt

SEQ ID NO: 26 - DNA, 11 bp, Artificial
  Synthetic K21
tttcccctccg

SEQ ID NO: 27 - DNA, 10 bp, Artificial
  Synthetic K32
ttgcctccgt

SEQ ID NO: 28 - DNA, 10 bp, Artificial
  Synthetic K37
tgtcgcctgt

SEQ ID NO: 29 - DNA, 11 bp, Artificial
  Synthetic K42
tcttcgctatc

SEQ ID NO: 30 - DNA, 10 bp, Artificial
  Synthetic K38
gcagttcact

SEQ ID NO: 31 - DNA, 10 bp, Artificial
  Synthetic K36
gcgttcatta

SEQ ID NO: 32 - DNA, 10 bp, Artificial
  Synthetic K39
tcttggggca

SEQ ID NO: 33 - DNA, 11 bp, Artificial
  Synthetic K44
tctgcgggaca

SEQ ID NO: 34 - DNA, 10 bp, Artificial
  Synthetic K40
acaagtctcg

SEQ ID NO: 35 - DNA, 11 bp, Artificial
  Synthetic K41
tatggacggct

SEQ ID NO: 36 - DNA, 11 bp, Artificial
  Synthetic K45
ttgtggtcgag

SEQ ID NO: 37 - DNA, 10 bp, Artificial
  Synthetic K50
acgccttcag

SEQ ID NO: 38 - DNA, 10 bp, Artificial
  Synthetic K51
gatttttggt

SEQ ID NO: 39 - DNA, 9 bp, Artificial
  Synthetic K52
gaggcggaa

SEQ ID NO: 40 - DNA, 10 bp, Artificial
  Synthetic K53
tctggcacat

SEQ ID NO: 41 - DNA, 10 bp, Artificial
  Synthetic K55
gaatgttttg

SEQ ID NO: 42 - DNA, 10 bp, Artificial
  Synthetic K56
tcgtttgtgt

SEQ ID NO: 43 - DNA, 10 bp, Artificial
  Synthetic K58
aggaggtgga

SEQ ID NO: 44 - DNA, 10 bp, Artificial
  Synthetic K59
tgtggcttag

SEQ ID NO: 45 - DNA, 12 bp, Artificial
  Synthetic K60
ccctcctactgt

SEQ ID NO: 46 - DNA, 11 bp, Artificial
  Synthetic K61
ttgcgccctgt

SEQ ID NO: 47 - DNA, 10 bp, Artificial
  Synthetic K63
gtgggtagta

SEQ ID NO: 48 - DNA, 10 bp, Artificial
  Synthetic K64
gagagtgggg

SEQ ID NO: 49 - DNA, 12 bp, Artificial
  Synthetic K70
ggtccgaggatc

SEQ ID NO: 50 - DNA, 10 bp, Artificial
  Synthetic K71
tattcttcgg

SEQ ID NO: 51 - DNA, 10 bp, Artificial
  Synthetic K73
agcgattgga

SEQ ID NO: 52 - DNA, 10 bp, Artificial
  Synthetic K74
tttgaggcct

SEQ ID NO: 53 - DNA, 10 bp, Artificial
  Synthetic K75
tctttggttt

SEQ ID NO: 54 - DNA, 11 bp, Artificial
  Synthetic K76
attgtccactg

SEQ ID NO: 55 - DNA, 9 bp, Artificial
  Synthetic K77
agaccggca

SEQ ID NO: 56 - DNA, 10 bp, Artificial
  Synthetic K78
tttgcggcta

SEQ ID NO: 57 - DNA, 10 bp, Artificial
  Synthetic K79
gttttgtgta

SEQ ID NO: 58 - DNA, 10 bp, Artificial
  Synthetic K80
atgccgtgat

SEQ ID NO: 59 - DNA, 10 bp, Artificial
  Synthetic K82
gagtggaagg

SEQ ID NO: 60 - DNA, 6 bp, Artificial
  Synthetic K85
gtcaca

SEQ ID NO: 61 - DNA, 10 bp, Artificial
  Synthetic K86
cttcctgttt

SEQ ID NO: 62 - DNA, 10 bp, Artificial
  Synthetic K87
tttgcggcta

SEQ ID NO: 63 - DNA, 11 bp, Artificial
  Synthetic K88
gatagtttaga

SEQ ID NO: 64 - DNA, 10 bp, Artificial
  Synthetic K89
gcaattatga

SEQ ID NO: 65 - DNA, 11 bp, Artificial
  Synthetic K90
taatggtgggt

SEQ ID NO: 66 - DNA, 10 bp, Artificial
  Synthetic K91
attgtacggg

SEQ ID NO: 67 - DNA, 8 bp, Artificial
  Synthetic K92
gtcggtca

SEQ ID NO: 68 - DNA, 10 bp, Artificial
  Synthetic K94
gctcttgctg

SEQ ID NO: 69 - DNA, 11 bp, Artificial
  Synthetic K95
ctttcctctct

SEQ ID NO: 70 - DNA, 10 bp, Artificial
  Synthetic K96
acagtgcagc

SEQ ID NO: 71 - DNA, 10 bp, Artificial
  Synthetic K97
gggtgacgtt

SEQ ID NO: 72 - DNA, 10 bp, Artificial
  Synthetic K98
acgcgtacct

SEQ ID NO: 73 - DNA, 10 bp, Artificial
  Synthetic K99
atcgtggtag

SEQ ID NO: 74 - DNA, 11 bp, Artificial
  Synthetic K101
gggtaggttgc

SEQ ID NO: 75 - DNA, 11 bp, Artificial
  Synthetic K102
tatacgcgcat

SEQ ID NO: 76 - DNA, 10 bp, Artificial
  Synthetic K103
cgtgagggat

SEQ ID NO: 77 - DNA, 10 bp, Artificial
  Synthetic K104
atttagcatt

SEQ ID NO: 78 - DNA, 11 bp, Artificial
  Synthetic K105
tcgatggnnnt

SEQ ID NO: 79 - DNA, 11 bp, Artificial
  Synthetic K107
tatggcacgaa

SEQ ID NO: 80 - DNA, 10 bp, Artificial
  Synthetic K108
gcgtccatgt

SEQ ID NO: 81 - DNA, 10 bp, Artificial
  Synthetic K109
gttggccggg

SEQ ID NO: 82 - DNA, 10 bp, Artificial
  Synthetic K110
tcgtaggctt

SEQ ID NO: 83 - DNA, 11 bp, Artificial
  Synthetic K111
gaccggcccct

SEQ ID NO: 84 - DNA, 10 bp, Artificial
  Synthetic K112
gcgccgttac

SEQ ID NO: 85 - DNA, 10 bp, Artificial
  Synthetic K113
gtgctgacat

SEQ ID NO: 86 - DNA, 10 bp, Artificial
  Synthetic K115
tccccgatcg

SEQ ID NO: 87 - DNA, 10 bp, Artificial
  Synthetic K116
gggtagagaa

SEQ ID NO: 88 - DNA, 10 bp, Artificial
  Synthetic K117
atttgtacgt

SEQ ID NO: 89 - DNA, 11 bp, Artificial
  Synthetic K118
gacgtctatgg

SEQ ID NO: 90 - DNA, 10 bp, Artificial
  Synthetic K119
agtggctggg

SEQ ID NO: 91 - DNA, 10 bp, Artificial
  Synthetic K120
gatggcggct

SEQ ID NO: 92 - DNA, 10 bp, Artificial
  Synthetic K121
tgcacatatt

SEQ ID NO: 93 - DNA, 10 bp, Artificial
  Synthetic K121
gttattgttc

SEQ ID NO: 94 - DNA, 10 bp, Artificial
  Synthetic K123
ggtaccgtta

SEQ ID NO: 95 - DNA, 10 bp, Artificial
  Synthetic K124
tatccctttg

SEQ ID NO: 96 - DNA, 11 bp, Artificial
  Synthetic K126
atagagccctt

SEQ ID NO: 97 - DNA, 10 bp, Artificial
  Synthetic K128
ttggttttgt

SEQ ID NO: 98 - DNA, 10 bp, Artificial
  Synthetic K129
ccgtgagcaa

SEQ ID NO: 99 - DNA, 10 bp, Artificial
  Synthetic K131
ggttgtagtt

SEQ ID NO: 100 - DNA, 10 bp, Artificial
  Synthetic K132
tccgtccgag

SEQ ID NO: 101 - DNA, 10 bp, Artificial
  Synthetic K133
cgtcctgcga

SEQ ID NO: 102 - DNA, 10 bp, Artificial
  Synthetic K134
gtatgggtgt

SEQ ID NO: 103 - DNA, 10 bp, Artificial
  Synthetic K137
tggtattgtg

SEQ ID NO: 104 - DNA, 10 bp, Artificial
  Synthetic K138
ggtccaaagt

SEQ ID NO: 105 - DNA, 10 bp, Artificial
  Synthetic K139
gtattgggca

SEQ ID NO: 106 - DNA, 10 bp, Artificial
  Synthetic K141
cctggacctt

SEQ ID NO: 107 - DNA, 11 bp, Artificial
  Synthetic K142
agatcagcgcg

SEQ ID NO: 108 - DNA, 10 bp, Artificial
  Synthetic K143
attagcctgg

SEQ ID NO: 109 - DNA, 9 bp, Artificial
  Synthetic K144
ggttatact

SEQ ID NO: 110 - DNA, 11 bp, Artificial
  Synthetic K145
tttgcggcgga

SEQ ID NO: 111 - RNA, 6 nt, Artificial
  Synthetic NCCNYN
gccccg

SEQ ID NO: 112 - RNA, 6 nt, Artificial
  Synthetic NSK1
gccucg

SEQ ID NO: 113 - RNA, 6 nt, Artificial
  Synthetic NSK1
gccgcg

SEQ ID NO: 114 - RNA, 6 nt, Artificial
  Synthetic NSK1
gccuug

SEQ ID NO: 115 - RNA, 6 nt, Artificial
  Synthetic NSK1
accgcg

SEQ ID NO: 116 - RNA, 6 nt, Artificial
  Synthetic NSK1
accacg

SEQ ID NO: 117 - RNA, 6 nt, Artificial
  Synthetic NSK1
accccg

SEQ ID NO: 118 - RNA, 6 nt, Artificial
  Synthetic NSK1
accuug

SEQ ID NO: 119 - RNA, 6 nt, Artificial
  Synthetic NSK1
gccccu

SEQ ID NO: 120 - RNA, 6 nt, Artificial
  Synthetic NSK1
gccgcu

SEQ ID NO: 121 - RNA, 6 nt, Artificial
  Synthetic NSK1
gccucu

SEQ ID NO: 122 - RNA, 6 nt, Artificial
  Synthetic NSK1
ucccug

SEQ ID NO: 123 - RNA, 6 nt, Artificial
  Synthetic NSK1
uccacg

SEQ ID NO: 124 - RNA, 6 nt, Artificial
  Synthetic NSK1
uccaug

SEQ ID NO: 125 - RNA, 6 nt, Artificial
  Synthetic NSK1
uccuug

SEQ ID NO: 126 - RNA, 6 nt, Artificial
  Synthetic NSK1
accgcc

SEQ ID NO: 127 - RNA, 6 nt, Artificial
  Synthetic NSK1
uccgcc

SEQ ID NO: 128 - RNA, 6 nt, Artificial
  Synthetic NSK1
cccgcu

SEQ ID NO: 129 - RNA, 6 nt, Artificial
  Synthetic NSK1
cccauc

SEQ ID NO: 130 - RNA, 6 nt, Artificial
  Synthetic NSK1
gccauc

SEQ ID NO: 131 - RNA, 6 nt, Artificial
  Synthetic NSK1
uccauc

SEQ ID NO: 132 - RNA, 6 nt, Artificial
  Synthetic NSK1
accauu

SEQ ID NO: 133 - RNA, 6 nt, Artificial
  Synthetic NSK1
cccauu

SEQ ID NO: 134 - RNA, 6 nt, Artificial
  Synthetic NSK1
uccacu

SEQ ID NO: 135 - RNA, 6 nt, Artificial
  Synthetic NSK1
gcccca

SEQ ID NO: 136 - RNA, 6 nt, Artificial
  Synthetic NSK1
gccccc

SEQ ID NO: 137 - RNA, 6 nt, Artificial
  Synthetic NSK1
accuca

SEQ ID NO: 138 - RNA, 6 nt, Artificial
  Synthetic NSK1
cccucc

SEQ ID NO: 139 - RNA, 6 nt, Artificial
  Synthetic NSK1
cccucg

SEQ ID NO: 140 - RNA, 6 nt, Artificial
  Synthetic NSK1
cccuuc

SEQ ID NO: 141 - RNA, 6 nt, Artificial
  Synthetic NSK1
accccc

SEQ ID NO: 142 - RNA, 6 nt, Artificial
  Synthetic NSK1
uccccc

SEQ ID NO: 143 - RNA, 6 nt, Artificial
  Synthetic NSK1
cccccc

SEQ ID NO: 144 - RNA, 6 nt, Artificial
  Synthetic NSK1
cccccu

SEQ ID NO: 145 - RNA, 6 nt, Artificial
  Synthetic NSK1
accucu

SEQ ID NO: 146 - RNA, 6 nt, Artificial
  Synthetic NSK1
ccccuc

SEQ ID NO: 147 - RNA, 6 nt, Artificial
  Synthetic NSK1
uggggc

SEQ ID NO: 148 - RNA, 6 nt, Artificial
  Synthetic NSK1
gggggg

SEQ ID NO: 149 - RNA, 6 nt, Artificial
  Synthetic NSK1
agggga

SEQ ID NO: 150 - RNA, 6 nt, Artificial
  Synthetic NSK1
ggaggg

SEQ ID NO: 151 - RNA, 6 nt, Artificial
  Synthetic NSK1
ggaggu

SEQ ID NO: 152 - RNA, 6 nt, Artificial
  Synthetic NSK1
ugcggc

SEQ ID NO: 153 - RNA, 6 nt, Artificial
  Synthetic NSK1
ugcggu

SEQ ID NO: 154 - RNA, 6 nt, Artificial
  Synthetic NSK1
ugcggg

SEQ ID NO: 155 - RNA, 6 nt, Artificial
  Synthetic NSK1
agcggg

SEQ ID NO: 156 - RNA, 6 nt, Artificial
  Synthetic NSK1
gaaggu

SEQ ID NO: 157 - RNA, 6 nt, Artificial
  Synthetic NSK1
agcggg

SEQ ID NO: 158 - RNA, 6 nt, Artificial
  Synthetic NSK1
ggcggg

SEQ ID NO: 159 - RNA, 6 nt, Artificial
  Synthetic NSK1
ugcggg

SEQ ID NO: 160 - RNA, 6 nt, Artificial
  Synthetic NSK1
gguggu

SEQ ID NO: 161 - RNA, 6 nt, Artificial
  Synthetic NSK1
gguggg

SEQ ID NO: 162 - RNA, 6 nt, Artificial
  Synthetic NSK1
uggggu

SEQ ID NO: 163 - RNA, 6 nt, Artificial
  Synthetic NSK1
aggggg

SEQ ID NO: 164 - RNA, 6 nt, Artificial
  Synthetic NSK1
gggggc

SEQ ID NO: 165 - RNA, 6 nt, Artificial
  Synthetic NSK1
gggggg

SEQ ID NO: 166 - RNA, 6 nt, Artificial
  Synthetic NSK1
caaggg

SEQ ID NO: 167 - RNA, 6 nt, Artificial
  Synthetic NSK1
gggggu

SEQ ID NO: 168 - RNA, 6 nt, Artificial
  Synthetic NSK1
cggggc

SEQ ID NO: 169 - RNA, 6 nt, Artificial
  Synthetic NSK1
ggcggu

SEQ ID NO: 170 - RNA, 6 nt, Artificial
  Synthetic NSK1
cgcggu

SEQ ID NO: 171 - RNA, 6 nt, Artificial
  Synthetic NSK1
cgcgga

SEQ ID NO: 172 - RNA, 6 nt, Artificial
  Synthetic NSK1
ugaggc

SEQ ID NO: 173 - RNA, 6 nt, Artificial
  Synthetic NSK1
agaggg

SEQ ID NO: 174 - RNA, 6 nt, Artificial
  Synthetic NSK1
gagggg

SEQ ID NO: 175 - RNA, 6 nt, Artificial
  Synthetic NSK1
gaggga

SEQ ID NO: 176 - RNA, 6 nt, Artificial
  Synthetic NSK2
uaggga

SEQ ID NO: 177 - RNA, 6 nt, Artificial
  Synthetic NSK2
uguggc

SEQ ID NO: 178 - RNA, 6 nt, Artificial
  Synthetic NSK2
gguggu

SEQ ID NO: 179 - RNA, 6 nt, Artificial
  Synthetic NSK2
aguggc

SEQ ID NO: 180 - RNA, 6 nt, Artificial
  Synthetic NSK2
cguggu

SEQ ID NO: 181 - RNA, 6 nt, Artificial
  Synthetic NSK2
gguggg

SEQ ID NO: 182 - RNA, 6 nt, Artificial
  Synthetic NSK2
aguggg

SEQ ID NO: 183 - RNA, 6 nt, Artificial
  Synthetic NSK2
aaugga

SEQ ID NO: 184 - RNA, 6 nt, Artificial
  Synthetic NSK2
gaaggg

SEQ ID NO: 185 - RNA, 6 nt, Artificial
  Synthetic NSK2
ggcggu

SEQ ID NO: 186 - RNA, 6 nt, Artificial
  Synthetic NSK2
ggcggg

SEQ ID NO: 187 - RNA, 6 nt, Artificial
  Synthetic NSK2
ggcgga

SEQ ID NO: 188 - RNA, 6 nt, Artificial
  Synthetic NSK2
uauggc

SEQ ID NO: 189 - RNA, 6 nt, Artificial
  Synthetic NSK2
gguggg

SEQ ID NO: 190 - RNA, 6 nt, Artificial
  Synthetic NSK2
agugga

SEQ ID NO: 191 - RNA, 6 nt, Artificial
  Synthetic NSK2
gggggu

SEQ ID NO: 192 - RNA, 6 nt, Artificial
  Synthetic NSK2
agggga

SEQ ID NO: 193 - RNA, 6 nt, Artificial
  Synthetic NSK2
ggaggg

SEQ ID NO: 194 - RNA, 6 nt, Artificial
  Synthetic NSK2
ugagga

SEQ ID NO: 195 - RNA, 6 nt, Artificial
  Synthetic NSK2
ugaggg

SEQ ID NO: 196 - RNA, 6 nt, Artificial
  Synthetic NSK2
gaaggc

SEQ ID NO: 197 - RNA, 6 nt, Artificial
  Synthetic NSK2
aaagga

SEQ ID NO: 198 - RNA, 6 nt, Artificial
  Synthetic NSK2
gggggg

SEQ ID NO: 199 - RNA, 6 nt, Artificial
  Synthetic NSK2
aggggg

SEQ ID NO: 200 - RNA, 6 nt, Artificial
  Synthetic NSK2
gggggu

SEQ ID NO: 201 - RNA, 6 nt, Artificial
  Synthetic NSK2
ugggga

SEQ ID NO: 202 - RNA, 6 nt, Artificial
  Synthetic NSK2
ugagga

SEQ ID NO: 203 - RNA, 6 nt, Artificial
  Synthetic NSK2
ugaggg

SEQ ID NO: 204 - RNA, 6 nt, Artificial
  Synthetic NSK2
uaggga

SEQ ID NO: 205 - RNA, 6 nt, Artificial
  Synthetic NS1
ugcucg

SEQ ID NO: 206 - RNA, 6 nt, Artificial
  Synthetic NS2
cgagca

SEQ ID NO: 207 - RNA, 6 nt, Artificial
  Synthetic NS3
acgagc

SEQ ID NO: 208 - RNA, 6 nt, Artificial
  Synthetic NS4
gcucgu

SEQ ID NO: 209 - RNA, 18 nt, Artificial
  Synthetic KG51
acgagcuggggcgcucgu

SEQ ID NO: 210 - RNA, 18 nt, Artificial
  Synthetic KC24 [Aptakiss]
ugcucggccccgcgagca

SEQ ID NO: 211 - DNA, 22 bp, Artificial
  Synthetic Adenoswitch
tgggggacuggggcgggaggaa

SEQ ID NO: 212 - DNA, 24 bp, Artificial
  Synthetic Adenoswitch TA
ttgggggacuggggcgggaggaaa

SEQ ID NO: 213 - DNA, 26 bp, Artificial
  Synthetic Adenoswitch TAGC
gttgggggacuggggcgggaggaaac

SEQ ID NO: 214 - RNA, 18 nt, Artificial
  Synthetic KC24 - Aptakiss
ugcucggccccgcgagca

SEQ ID NO: 215 - RNA, 8 nt, Artificial
  Synthetic KG51 loop sequence
cuggggcg

SEQ ID NO: 216 - RNA, 18 nt, Artificial
  Synthetic KC24 [Aptakiss]
ugcucggccccgcgagca

SEQ ID NO: 217 - RNA, 45 nt, Artificial
  Synthetic Oligonucleotides containing 10 or 11 random nucleotides
gguuaccagccuucacugcucgncgagcaccacggucggucacac

SEQ ID NO: 218 - RNA, 18 nt, Artificial
  Synthetic Oligonucleotide a consensus motif flanked by invariant primer annealing sites
acgagcnrnggngcucgu

SEQ ID NO: 219 - RNA, 50 nt, Artificial
  Synthetic Oligonucleotide containing a consensus motif flanked by invariant primer annealing sites
gguuaccagccuucacugcucgnccnnncgagcaccacggucggucacac

SEQ ID NO: 220 - RNA, 49 nt, Artificial
  Synthetic Oligonucleotide containing a consensus motif flanked by invariant primer annealing sites
gggaggacgaagcggacgagcnrnggngcucgucagaagacacgcccga

SEQ ID NO: 221 - DNA, 36 bp, Artificial
  Synthetic primer
taatacgactcactataggttaccagccttcactgc

SEQ ID NO: 222 - DNA, 32 bp, Artificial
  Synthetic primer
taatacgactcactatagggaggacgaagcgg

SEQ ID NO: 223 - DNA, 16 bp, Artificial
  Synthetic primer
tcgggcgtgtcttctg

SEQ ID NO: 224 - RNA, 20 nt, Artificial
  Synthetic K18 (long)
ugcucgacgcguccucggca

SEQ ID NO: 225 - RNA, 18 nt, artificial
  Synthetic Kx3
ggucggucccagacgacc

SEQ ID NO: 226 - RNA, 38 nt, artificial
  Synthetic Kx4
gguuucagggcagugauguugccccucggaagauaacc

SEQ ID NO: 227 - RNA, 16 nt, artificial
  Synthetic Kx3′
cgagccugggagcucg

SEQ ID NO: 228 - RNA, 15 nt, artificial
  Synthetic Kx4′
ccugacaucaccagg

SEQ ID NO: 229 - RNA, 35 nt, artificial
  Synthetic GTPswitch
uccgaagugguugggcuggggcgugugaaaacgga

SEQ ID NO: 230 - RNA, 18 nt, artificial
  Synthetic aptakissmut
ugcucggccgcgcgagca

SEQ ID NO: 231 - DNA, 22 bp, artificial
  Synthetic adenoswitchmut
tgggggacugcggcgggaggaa

(19) United States
(12) Patent Application Publication
(10) Pub. No.: US 2016/0274095 A1
(43) Pub. Date: Sep. 22, 2016

(54) Kits-of-Parts Comprising Nucleic Acids Able to Form a Kissing Complex and Their uses Thereof

(71) Applicants: INSERM (INSTITUT NATIONAL DE LA SANTE ET DE LA RECHERCHE MEDICALE, Paris (FR); UNIVERSITE DE BORDEAUX, Bordeaux (FR); Centre National de la Recherche Scientifique (CNRS, Paris (FR); Universite Grenoble Alpes, Grenoble (FR)

(72) Inventors: Jean-Jacques Toulme, Lormont (FR); Eric Dausse, Bordeaux (FR); Guillaume Durand, Bordeaux (FR); Eric Peyrin, Saint-Egreve (FR); Corinne Ravelet, Lancey (FR)

(21) Appl. No.: 15/036,558

(22) PCT Filed: Nov. 13, 2014
(86) PCT No.: PCT/EP2014/074548

§ 371 Date: May 13, 2016

(30) Foreign Application Priority Data
Nov. 13, 2013 (EP) 13306546.6

(51) Int. Cl. G01N 33/53; C12N 15/10; C07H 21/02; C12N 15/115

(52) U.S. Cl. CPC G01N 33/5308; C12N 15/115; C12N 15/1048; C07H 21/02; C12N 2310/16; C12N 2310/531

(57) ABSTRACT

The present invention relates to a kit-of-parts comprising at least one nucleic acid molecule NA1 and at least one nucleic acid molecule NA2 wherein the nucleic acid molecules NA1 and NA2 are capable of forming duplexes via the formation of a kissing complex. The present invention also describes use of such kit-of-parts for detecting target molecules of interest but also for selecting aptamers of interest in solution.

### FIELD OF THE INVENTION

The present invention relates to kits-of-parts comprising nucleic acids able to form a kissing complex and their uses thereof.

### BACKGROUND OF THE INVENTION

Aptamers are DNA or RNA oligomers selected from random pools on the basis of their ability to bind other molecules (Ellington et al (1990) Nature 346 (6287): 818, Robertson and Joyce (1990) Nature 344 (6265): 467, Tuerk and Gold (1990) Science 249 (4968): 505). To date, aptamers have been selected against many different types of targets: small organic compounds, proteins, nucleic acids and complex scaffolds such as live cells (Dausse et al. (2009) Curr. Opin. Pharmacol 9(5): 602, Hall et al. (2009) Curr. Protoc. Mol. Biol. Chapter 24, Unit 24 (3)). These molecules rival with antibodies in terms of binding properties, specificity of recognition and potential uses in medicine and technology. Aptamers are generally obtained by systematic evolution of ligands by exponential enrichment (SELEX) (Gold et al. (1997) Proc. Natl. Acad. Sci. USA 94 (1): 89) even though selection without any amplification step (non-SELEX) has also been described (Berezovski M et al. (2006) J Am Chem Soc. 2006 Feb. 8; 128(5):1410-1, Javaherian et al. (2009) Nucleic Acids Res 37 (8): e62).

Selection of RNA candidates to RNA hairpins led to hairpin aptamers whose loop is complementary to that of the target hairpin thus generating loop-loop interaction. The stability of such so-called kissing complexes originates in Watson Crick base pairs of loop-loop helix but also in stacking interactions at the junctions between the loop-loop module and the double stranded stem of each hairpin partner. Indeed the binding of the Trans-Activating Responsive (TAR) RNA imperfect stem loop element of the Human Immunodeficiency Virus to a hairpin aptamer generating a 6 base pair loop-loop helix was characterized by a melting temperature 20° C. higher than that of the complex between TAR and an antisense oligomer giving rise to the same 6 base pair duplex. However the potential of hairpins to discriminate between folded and linear structures for melting temperature 20° C. higher than that of the complex between has not yet been exploited.

### SUMMARY OF THE INVENTION

The present invention relates to a kit-of-parts comprising at least one nucleic acid molecule NA1 and at least one nucleic acid molecule NA2 wherein the nucleic acid molecules NA1 and NA2 are capable of forming duplexes via the formation of a kissing complex. The present invention also describes the use of such kit-of-parts for detecting target molecules of interest but also for selecting aptamers of interest in solution.

### DETAILED DESCRIPTION OF THE INVENTION

### Kits-of-Parts

The present invention relates to a kit-of-parts comprising at least one nucleic acid molecule NA1 and at least one nucleic acid molecule NA2 wherein:

a) the first nucleic acid molecule NA1 comprises the nucleotide acid sequence of NS1-NSK1-NS2, wherein
NS1 and NS2 consist of polynucleotides having at least 1 nucleotide in length, and NS1 and NS2 have complementary sequences;
NSK1 has a nucleotide acid sequence of at least 2 nucleotides,

b) the second nucleic acid molecule NA2 comprises the nucleotide sequence of NS3-NSK2-NS4 wherein:
NS3 and NS4 consist of polynucleotides having at least 1 nucleotide in length, and NS3 and NS4 have complementary sequences;
NSK2 has a nucleotide acid sequence of at least 2 nucleotides

c) the nucleic acid molecules (NA1 and NA2) are both capable to form in appropriate conditions at least one hairpin loop comprising the sequences NSK1 and NSK2 respectively and

d) the nucleic acid molecules NA1 and NA2 are able to form a duplex by the formation of a kissing complex between the hairpin loops comprising the sequences NSK1 and NSK2 respectively.

As used herein the terms “nucleotide” has its general meaning in the art and includes, but is not limited to, a natural nucleotide, a synthetic nucleotide, or a nucleotide analogue. The nucleoside phosphate may be a nucleoside monophosphate, a nucleoside diphosphate or a nucleoside triphosphate. The sugar moiety in the nucleoside phosphate may be a pentose sugar, such as ribose, and the phosphate esterification site may correspond to the hydroxyl group attached to the C-5 position of the pentose sugar of the nucleoside. A nucleotide may be, but is not limited to, a deoxyribonucleoside triphosphate (dNTP) or a ribonucleoside triphosphate (NTP). The nucleotides may be represented using alphabetical letters (letter designation), as described in Table A. For example, A denotes adenosine (i.e., a nucleotide containing the nucleobase, adenine), C denotes cytosine, G denotes guanosine, and T denotes thymidine. W denotes either A or T/U, and S denotes either G or C. N represents a random nucleotide (i.e., N may be any of A, C, G, or T/U). As used herein, the term “nucleotide analogue” refers to modified compounds that are structurally similar to naturally occurring nucleotides. The nucleotide analogue may have an altered phosphorothioate backbone, sugar moiety, nucleobase, or combinations thereof. Generally, nucleotide analogues with altered nucleobases confer, among other things, different base pairing and base stacking properties. Nucleotide analogues having altered phosphate-sugar backbone (e.g., PNA, LNA, etc.) often modify, among other things, the chain properties such as secondary structure formation. At times in the instant application, the terms “nucleotide analogue,” “nucleotide analogue base,” “modified nucleotide base,” or “modified base” may be used interchangeably.

TABLE A
letter designations of various nucleotides

| Symbol Letter | Nucleotide |
|---|---|
| G | Guanosine |
| A | Adenosine |
| T | Thymidine |
| C | Cytosine |
| U | Uracil |
| R | G or A |
| Y | T, U, or C |
| N | G, A, T, U or C |
| W | A, T/U |
| S | G/C |

As used herein, the term “hairpin loop” is meant to refer to a feature of ribonucleic acid (RNA) secondary structure. A hairpin loop occurs when RNA folds back on itself. Base pairing along the double-stranded stems may be either perfectly complementary or may contain mismatches.

As used herein, the term “kissing complex” is meant to refer to the base-pairing between complementary sequences in the apical loops of two hairpins which is a basic type of RNA tertiary contact (Lee et al., Structure 6:993-1005.1998). This complex facilitates the pairing of hairpin loops permitting the two nucleic acid molecules to form a duplex. Molecular dynamics, liquid-crystal NMR spectroscopy and X-ray crystallography showed that the tridimensional structure of the kissing complex is characterized by: i) quasi-continuous stacking from one stem to the other through the intermolecular loop-loop helix, ii) two phosphate clusters flanking the major groove of the loop-loop helix that likely constitute the binding sites for magnesium ions that were shown to be crucial for stability, iii) non canonical interactions such as stacking interactions and interbackbone H-bond network. According to the invention, the kissing complex is formed between the pair of hairpin loops which comprise sequences NSK1 and NSK2 respectively.

As used herein the expression “appropriate conditions” refer to any condition that favour the formation of a kissing complex as above defined. In particular, the appropriate conditions refer to the conditions under which the nucleic acids NA1 and NA2 are correctly folded (i.e. the hairpin loop comprising the sequence comprising the sequences NSK1 and NSK2 respectively are correctly formed).

In some embodiments, a kit-of-parts according to the invention comprises a first nucleic acid molecule NA1 folded in a hairpin structure wherein NSK1 is represented by sequence loops able to interact with a second nucleic acid sequences NSK2 present in the loop of a second acid nucleic acid molecule NA2 folded in an hairpin structure.

In some embodiments, NKS1 has a nucleotide acid sequence of 2, 3, 4, 5, 6, 7, 8, 9, 10, 11, 12, 13, 14, or 15 nucleotides.

In some embodiments, NKS2 has a nucleotide acid sequence of 2, 3, 4, 5, 6, 7, 8, 9, 10, 11, 12, 13, 14, or 15 nucleotides.

In some embodiments, a kit-of-parts according to the invention comprises a first nucleic acid molecule NA1 wherein NSK1 has a sequence selected from the group consisting of YRYR, RYRY, YYRY, RYRR, YYYR, YRYY, RYYR, YRRY, YRRR, RYYY, RRYR, RRYY, RRRR, RRRY, YYYY, YYRR and a second nucleic acid molecule NA2 wherein NKS2 is able to form a kissing complex with NKS1.

In some embodiments, a kit-of-parts according to the invention comprises a first nucleic acid molecule NA1 wherein NSK1 is represented by Kₙ and a second nucleic acid molecule NA2 wherein NKS2 is represented Kₙ′, wherein Kₙ and Kₙ′ are selected as depicted in Table B (Kₙ and Kₙ′ may be identical or not).

TABLE B
description of possible couples between Kₙ and Kₙ′

| Kₙ is | Kₙ′ is selected from the group consisting of |
|---|---|
| K1 | K11, K14, K15, K1, K28, K34, K39, K51, K52, K53, K59, K61, K73, K75, and K85 |
| K10 | K13, K25, K29, K37, K4, K60bis, K61, K74, K86, and K94, |
| K11 | K14, K17, K1, K30, K37, K44, K52, K61, K78, K82, K86, K87, and K98 |
| K13 | K10, K18, K24, K35, K36, K50, K73, K96, and K98 |
| K15 | K1, K76, and K89 |
| K16 | K24, K30, K36, K39, K52, K63, K90, and K98 |
| K17 | K11, K17, K20, K38, K39, K8, and K96 |
| K18 | K14, K16, K18, K24, K2, K34, K36, K41, K44, K50, K58, K5, K70, K97, and K98 |
| K2 | K14, K18, K24, K2, K36, K45, K52, K78, K87, and K98 |
| K20 | K16, K17, K5, K60bis, K61, K76, and K98 |
| K21 | K39, K44, K52, K58, K64, K82, and K8 |
| K24 | K14, K18, K24, K2, K41, K42, K50, K52, K5, K77, K78, K87, and K92bis |
| K25 | K10, K34, K41, and K44 |
| K27 | K27, K2, K32, K5, K76, K80, K91, and K98 |
| K28 | K11, K14, K1, K28, K35, K61, and K89 |
| K29 | K10, K50, K59, and K77 |
| K30 | K11, K16, K1, K30, K52, K61, K74, and K89 |
| K32 | K14, K27, K39, K52, K53, K58, K74, K77, K89, K91, K92bis, and K9 |
| K34 | K18, K25, K55, K76, K86, and K89 |
| K35 | K13, K28, K42, K59, K5, K75, K88, and K95 |
| K36 | K13, K14, K16, K18, K2, K50, K5, K89, K98, and K9 |
| K37 | K10, K11, K34, K44, K52, K73, K74, and K9 |
| K38 | K17, K44, K4, K60bis, K64, K76, and K82 |
| K39 | K16, K17, K1, K32, K5, K61, and K80 |
| K4 | K10, K38, K76, and K85 |
| K40 | K45, K56, K64, K70, K76, K77, K79, and K94 |
| K41 | K18, K24, K25, K70, and K76 |
| K42 | K35, K73, K88, and K8 |
| K44 | K10, K11, K18, K24, K25, K2, K38, K5, and K76 |
| K45 | K2, K40, K53, K5, K6, K76, K77, and K85 |
| K5 | K13, K24, K35, K39, K45, K4, K64, K73, K80, K82, K8, K90, and K99 |
| K50 | K13, K18, K24, K29, K36, K52, K74, K82, and K98 |
| K51 | K1, K55, and K80 |
| K52 | K11, K16, K1, K21, K24, K25, K2, K30, K32, K37, K50, K60bis, K61, and K74 |
| K53 | K10, K1, K45, K55, K56, K59, K79, K80, and K96 |
| K55 | K34, K51, K53, K6, and K71 |
| K56 | K16, K53, K85, and K9 |
| K58 | K18, K21, K32, K60bis, K76, K86, and K98 |
| K59 | K1, K24, K29, K35, K53, K5, K76, and K85 |
| K6 | K36, K45, K6, and K89 |
| K60bis | K20, K70, K74, K96, and K99 |
| K61 | K11, K14, K1, K20, K28, K30, K52, K61, K89, and K8 |
| K63 | K16, K5, K76, and K98 |
| K64 | K16, K38, K40, K5, K76, K94, and K95 |
| K70 | K14, K18, K21, K2, K32, K34, K40, K41, K52, K71, K77, K92bis, and K95 |
| K71 | K10, K55, K70, and K82 |
| K73 | K13, K1, K28, K37, K42, K5, K76, K89, and K99 |
| K74 | K10, K21, K30, K32, K50, K52, K60bis, and K74, |
| K76 | K15, K20, K27, K34, K38, K3, K40, K41, K44, K45, K58, K59, K63, K64, K73, K82, |
| | K89, K90, K96, K99, and K9 |
| K77 | K24, K29, K40, K45, K70, K77, K80, and K9 |
| K78 | K11, K24, K2, K80, and K89 |
| K79 | K40, K53, and K85 |
| K8 | K17, K21, K42, and K61 |
| K80 | K27, K38, K39, K41, K51, K53, K5, K77, K78, K85, K87, K91, K92bis, and K9 |
| K82 | K11, K21, K38, K50, K5, K71, K76, K86, and K95 |
| K85 | K2, K45, K4, K56, K59, K79, K80, and K97 |
| K86 | K11, K58, and K82 |
| K87 | K11, K24, K2, K80, and K89 |
| K89 | K15, K28, K32, K36, K61, K6, K73, K76, K78, K87, K90, K91, and K94 |
| K9 | K18, K25, K32, K56, K76, K79, K80, K91, and K97 |
| K90 | K16, K36, K5, K76, and K89 |
| K91 | K27, K32, K89, K91, and K98 |
| K92bis | K24, K32, K70, and K80 |
| K94 | K40, K64, and K96 |
| K95 | K34, K35, K64, K70 and K82 |
| K96 | K13, K17, K25, K37, K38, K53, K60bis, K61, K76, K86, and K94 |
| K97 | K38, K85, K97, and K9 |
| K98 | K11, K13, K14, K16, K18, K24, K27, K2, K36, K50, K5, K91, and K98 |
| K99 | K5, K60bis, K73, K76, and K98 |

In some embodiments, a kit-of-parts according to the invention comprises a first nucleic acid molecule NA1 wherein NSK1 is represented by Kₙ and a second nucleic acid molecule NA2 wherein NKS2 is represented Kₙ′, wherein Kₙ and Kₙ′ are selected as depicted in Table C1 (Kₙ and Kₙ′ may be identical or not).

TABLE C1
description of the best couples between Kn and Kn′

| Kₙ is | Kₙ′ is |
|---|---|
| K1 | K39 |
| K10 | K13 |
| K10 | K4 |
| K11 | K44 |
| K13 | K10 |
| K14 | K14 |
| K15 | K76 |
| K16 | K30 |
| K17 | K8 |
| K18 | K18 |
| K18 | K24 |
| K18 | K98 |
| K2 | K36 |
| K20 | K17 |
| K20 | K24 |
| K20 | K76 |
| K21 | K58 |
| K21 | K8 |
| K24 | K78 |
| K24 | K87 |
| K25 | K41 |
| K27 | K76 |
| K28 | K52 |
| K29 | K59 |
| K3 | K32 |
| K3 | K76 |
| K30 | K30 |
| K30 | K70 |
| K32 | K58 |
| K34 | K18 |
| K34 | K25 |
| K35 | K13 |
| K35 | K28 |
| K35 | K42 |
| K35 | K5 |
| K36 | K2 |
| K37 | K97 |
| K38 | K41 |
| K39 | K1 |
| K39 | K70 |
| K4 | K10 |
| K40 | K44 |
| K41 | K25 |
| K42 | K52 |
| K42 | K8 |
| K44 | K11 |
| K45 | K2 |
| K5 | K64 |
| K50 | K58 |
| K51 | K70 |
| K52 | K21 |
| K52 | K28 |
| K53 | K1 |
| K55 | K44 |
| K56 | K41 |
| K58 | K21 |
| K59 | K24 |
| K6 | K45 |
| K6 | K4 |
| K6 | K97 |
| K60bis | K82 |
| K60bis | K8 |
| K61 | K8 |
| K63 | K76 |
| K64 | K5 |
| K70 | K70 |
| K71 | K70 |
| K73 | K24 |
| K74 | K74 |
| K75 | K70 |
| K76 | K27 |
| K76 | K63 |
| K77 | K44 |
| K77 | K80 |
| K78 | K24 |
| K79 | K44 |
| K8 | K21 |
| K80 | K99 |
| K82 | K95 |
| K85 | K59 |
| K86 | K8 |
| K87 | K24 |
| K88 | K25 |
| K89 | K99 |
| K9 | K76 |
| K90 | K21 |
| K91 | K17 |
| K92bis | K80 |
| K92bis | K92bis |
| K94 | K11 |
| K95 | K82 |
| K96 | K60bis |
| K97 | K37 |
| K98 | K18 |
| K98 | K98 |
| K99 | K5 |

In some embodiments, a kit-of-parts according to the invention comprises a first nucleic acid molecule NA1 wherein NSK1 comprises a nucleic acid sequence consisting of CCNY and a second nucleic acid molecule NA2 wherein NKS2 comprises a nucleic acid sequence consisting of RNGG.

In some embodiments, a kit-of-parts according to the invention comprises a first nucleic acid molecule NA1 wherein NSK1 comprises a nucleic acid sequence consisting of NCCNYN and a second nucleic acid molecule NA2 wherein NKS2 comprises a nucleic acid sequence consisting of NRNGGN.

In some embodiments, a kit-of-parts according to the invention comprises a first nucleic acid molecule NA1 wherein NSK1 comprises a nucleic acid sequence consisting of NCCNYN and a second nucleic acid molecule NA2 wherein NKS2 comprises a nucleic acid sequence consisting of NRNGGN, wherein sequences NCCNYN and sequence NRNGGN are respectively selected as depicted in Table C2.

TABLE C2
description of NCCNYN/NRNGGN possible couples

| NCCNYN is selected from the group | |
|---|---|
| consisting of | NRNGGN is selected from the group consisting of |
| GCCCCG | UGGGGC GGGGGG and AGGGGA |
| GCCUCG | GGAGGG and GGAGGU |
| GCCGCG | UGCGGC UGCGGU UGCGGG and AGCGGG |
| GCCUUG | GAAGGU |
| ACCGCG | AGCGGG GGCGGG and UGCGGG |
| ACCACG | GGUGGU and GGUGGG |
| ACCCCG | UGGGGU AGGGGG GGGGGC and GGGGGG |
| ACCUUG | CAAGGG |
| GCCCCU | GGGGGU and CGGGGC |
| GCCGCU | GGCGGU CGCGGU and CGCGGA |
| GCCUCU | UGAGGC and AGAGGG |
| UCCCUG | GAGGGG GAGGGA and UAGGGA |
| UCCACG | UGUGGC GGUGGU AGUGGC CGUGGU |
| | GGUGGG and AGUGGG |
| UCCAUG | AAUGGA |
| UCCUUG | GAAGGG |
| ACCGCC UCCGCC and CCCGCU | GGCGGU GGCGGG and GGCGGA |
| CCCAUC GCCAUC UCCAUC ACCAUU | UAUGGC |
| and CCCAUU | |
| UCCACU | GGUGGG and AGUGGA |
| GCCCCA GCCCCC | GGGGGU and AGGGGA |
| ACCUCA CCCUCC CCCUCG | GGAGGG UGAGGA and UGAGGG |
| CCCUUC | GAAGGC and AAAGGA |
| ACCCCC UCCCCC CCCCCC CCCCCU | GGGGGG AGGGGG GGGGGU and UGGGGA |
| ACCUCU | UGAGGA and UGAGGG |
| CCCCUC | UAGGGA |

In some embodiments, NS1, NS2, NS3 or NS4 comprises 1, 2, 3, 4, 5, 6, 7, 8, 9, 10, 11, 12, 13, 14 or 15 nucleotides.

In some embodiments, NS1 is represented by UGCUCG and NS2 is represented by CGAGCA.

In some embodiments, NS3 is represented by ACGAGC and NS4 is represented GCUCGU.

In some embodiments, the loop of the nucleic acid comprises the D21 DNA loop, in particular as provided in the EXAMPLES.

In some embodiments, a kit-of-parts according to the invention comprises a first nucleic acid molecule comprising a nucleic acid sequence as set forth by ACGAGCUGGGGCGCUCGU (KG51) and second nucleic acid molecule comprising a nucleic acid sequence as set forth by UGCUCGGCCCCGCGAGCA (KC24-Aptakiss).

In some embodiments, a kit-of-parts according to the invention comprises a first nucleic acid molecule comprising a nucleic acid sequence as set forth by TGGGGGACUGGGGCGGGAGGAA and a second nucleic acid molecule comprising a nucleic acid sequence as set forth by UGCUCGGCCCCGCGAGCA (KC24-Aptakiss).

In some embodiments, a kit-of-parts according to the invention comprises a first nucleic acid molecule consisting of a nucleic acid sequence as set forth by TTGGGGGACUGGGGCGGGAGGAAA and second nucleic acid molecule consisting of a nucleic acid sequence as set forth by UGCUCGGCCCCGCGAGCA (KC24-Aptakiss).

In some embodiments, a kit-of-parts according to the invention comprises a first nucleic acid molecule consisting of a nucleic acid sequence as set forth by GTTGGGGGACUGGGGCGGGAGGAAAC and second nucleic acid molecule consisting of a nucleic acid sequence as set forth by UGCUCGGCCCCGCGAGCA (KC24-Aptakiss).

In some embodiments, at least one nucleic acid molecule is an aptamer, i.e. a nucleic acid molecule that exhibit specificity and affinity for a target molecule, so that the RNA loop part of this aptamer could be any nucleic acid sequence able to form a kissing complex with the second nucleic acid hairpin.

In some embodiments, the NSK1 and/or NSK2 sequence (i.e. the sequence forming the loop of the molecule) is a DNA or RNA nucleic acid sequence.

As used herein, “specificity” refers to the ability of the nucleic acid molecule to distinguish in a reasonably unique way between the target molecule and any other molecules.

The “affinity” of the nucleic acid molecule for its target molecule corresponds to stability of the complex between the two and can be expressed as the equilibrium dissociation constant (KD). The techniques used to measure affinity are well-known by the skilled person. They can be, for example Surface Plasmon Resonance. The affinity depends on the nature of the nucleic acid molecule and of the target molecule. The one skilled in the art is able to determine the desired conditions depending on the tested nucleic acid molecules and target molecules. More precisely, the one skilled in the art is able to define the sufficient level of affinity for obtaining the desired aptamers.

Particularly, the aptamer can be used for targeting various organic and inorganic materials or molecules. Typically the aptamer is specific for any kind of target such as, nucleic acid molecules, lipids, microorganisms, viruses, oligopeptides, polypeptides proteins, polymers, macromolecules, small organic molecules . . . .

In some embodiments, the aptamer is specific for a small organic molecule. The term “small organic molecule” refers to a molecule of a size comparable to those organic molecules generally used in pharmaceuticals. The term excludes biological macromolecules (e.g., proteins, nucleic acids, etc.). Preferred small organic molecules range in size up to about 5000 Da, more preferably up to 2000 Da, and most preferably up to about 1000 Da.

In some embodiments, the aptamer is specific for a small organic molecule which contains at least one aromatic ring group. As used herein, the term “aromatic ring group” may refer to a group where electrons are delocalized or resonaned, and examples may include an aryl group, a heteroaryl group, and the like.

In some embodiments, the aptamer that binds a small organic molecule undergoes conformational changes upon interactions with the small organic molecule, thus permitting the formation of the hairpin loop that is able to form the kissing complex. Accordingly, in absence of the small organic molecule, the aptamer is not able to form a heterodimer via the formation of the kissing complex, while in presence of the small organic molecule the aptamer adopts conformation changes and thus is able to form a heterodimer via the formation of the kissing complex.

In some embodiments, the aptamer derives from a previously known aptamer (i.e. a primary aptamer) which has been raised against the target molecule. As used herein the term “derives” means that the primary aptamer has been modified to include a sequence as described herein that is able to form a kissing complex. Typically, the previous primary aptamer is converted to the secondary aptamer of the kit-of-parts by substituting a sequence of a hairpin loop of the previous known aptamer (e.g. which forms the apical part of the previously known aptamer) with a sequence as described herein that is able to form a kissing complex. The EXAMPLE 2 describes one example in which a primary aptamer is converted to a secondary aptamer according to the invention.

In some embodiments, the aptamer of the invention is preferably a synthetic nucleic acid molecule selected by the SELEX method from an underlying synthetic combinatorial library. Indeed one skilled in the art may perform the known SELEX method under the usual conditions and with a suitable affinity, i.e. to obtain a candidate enriched mixture containing the nucleic acid molecules having a strong affinity (those having the strongest affinity in the starting mixture). Accordingly, the SELEX method involves the combination of a selection of nucleic acid candidates which all contain a sequence as described herein that is able to form a kissing complex and which bind to a target molecule with an amplification of those selected nucleic acids. Iterative cycling of the selection/amplification steps allows selection of nucleic acids which bind most strongly to the target from a pool which contains a very large number of nucleic acids. For example, the SELEX method (hereinafter termed SELEX), was first described in U.S. application Ser. No. 07/536,428, filed Jun. 11, 1990, entitled “Systematic Evolution of Ligands By Exponential Enrichment,” now abandoned. U.S. Pat. No. 5,475,096, entitled “Nucleic Acid Ligands,” and U.S. Pat. No. 5,270,163, entitled “Methods for Identifying Nucleic Acid Ligands,” also disclose the basic SELEX process.

The SELEX-type process as used in a method according to the invention may, for example, be defined by the following series of steps:

i) Contacting a mixture of candidate nucleic acids which all contain a sequence as described herein that is able to form a kissing complex with the target molecule; nucleic acids having a strongest affinity to the target molecule relative to the candidate mixture may be partitioned from the remainder of the candidate nucleic acid mixture. Preferably, the mixture is contacted with the selected target molecule under conditions suitable for binding to occur between them. Under these circumstances, complexes between the target molecule and the nucleic acids having the strongest affinity for the target molecule can be formed.

ii) Partitioning the nucleic acids with the strongest affinity for the target molecule from the remainder of the candidate mixture. At this step, the nucleic acids with the strongest affinity for the target molecule are partitioned from those nucleic acids with lesser affinity to the target molecule.

iii) Amplifying the nucleic acids with the strongest affinity to the target molecule to yield a candidate enriched mixture of nucleic acids. In this step, those nucleic acids selected during partitioning as having a relatively higher affinity to the target molecule are amplified to create a new candidate mixture that is enriched in nucleic acids having a relatively higher affinity for the target.

In some embodiments, the partitioning and amplifying steps above can be repeated (cycling) so that the newly formed candidate mixture contains fewer unique sequences and the average degree of affinity of the nucleic acid mixture to the target is increased.

“Partitioning” means any process whereby nucleic acid candidates bound to target molecules, identified herein as candidate-target complexes, can be separated from nucleic acids not bound to target molecules. Partitioning can be accomplished by various methods known in the art. For example, candidate-target complexes can be bound to nitrocellulose filters while unbound candidates are not. Columns which specifically retain candidate-target complexes can be used for partitioning. Liquid-liquid partition can also be used as well as filtration gel retardation, affinity chromatography and density gradient centrifugation. Alternatively, the partitioning can be performed by attaching the target molecules on magnetic beads followed by binding of the nucleic acids to the target molecules and subsequent separation of the magnetic beads/target molecules/nucleic acids particles. Several different methods of automated separation of magnetic beads are known from the art. The first method is to insert a magnetic or magnetizable device into the medium containing the magnetic beads, binding the magnetic beads to the magnetic or magnetizable device, and remove the magnetic or magnetizable device. In a second method the separation of medium and the magnetic particles, both aspirated into a pipette tip, is facilitated by a magnetic or magnetizable device which is brought into spatial proximity to the pipette tip. The choice of the partitioning method will depend on the properties of the target and of the candidate-target complexes and can be made according to principles known to those of ordinary skill in the art.

After the candidate nucleic acids bound to the target molecules have been separated from those which have remained unbound, the next step in partitioning is to separate them from the target molecules. Thus, the candidate nucleic acids can be separated by heating in water at a temperature sufficient to allow separation of the species. Alternatively separation can be achieved by addition of a denaturing agent or a degrading agent, for instance an enzyme. Bound candidates can also be collected by competition with the free target. For example, the candidate nucleic acids can be separated by heating in water for one minute at 75° C. A mixture of nucleic acids with increased affinity to the target molecule is thus obtained.

After partitioning, the candidate nucleic acids with high affinity may be amplified. As intended herein “amplifying” means any process or combination of process steps that increases the amount or number of copies of a molecule or class of molecules.

The amplification step can be performed by various methods which are well known to the person skilled in the art. A method for amplifying DNA molecules can be, for example, the polymerase chain reaction (PCR). In its basic form, PCR amplification involves repeated cycles of replication of a desired single-stranded DNA (or cDNA copy of an RNA) using specific oligonucleotides complementary to the 3′ and 5′ ends of the single stranded DNA as primers, achieving primer extension with a DNA polymerase followed by DNA denaturation. The products generated by extension from one primer serve as templates for extension from the other primer. Descriptions of PCR methods are found in Saiki et al. (1985) Science 230:1350-1354 or Saiki et al. (1986) Nature 324:163-166. Methods for amplifying RNA molecules are well known from the person skilled in the art. For example, amplification can be carried out by a sequence of three reactions: making cDNA copies of selected RNAs (using reverse transcriptase), using the polymerase chain reaction to increase the copy number of each cDNA, and transcribing the cDNA copies to obtain RNA molecules having the same sequences as the selected RNAs. In accordance with the invention, the candidate nucleic acids are preferably amplified with the help of oligonucleotides capable of hybridizing to fixed sequences common to these nucleic acids. In accordance with the invention, an amplification step is preferentially carried out on the mixture of nucleic acids with increased affinity obtained during the partitioning step to yield a candidate enriched mixture of nucleic acids. The relative concentrations of target molecules to nucleic acid employed to achieve the desired partitioning will depend for example on the nature of the target molecule, on the strength of the binding interaction and on the buffer used. The relative concentrations needed to achieve the desired partitioning result can be readily determined empirically without undue experimentation.

Cycling (repetition) of the partitioning/amplification procedure can be continued until a selected goal is achieved. For example, cycling can be continued until a desired level of binding of the nucleic acids in the test mixture is achieved or until a minimum number of nucleic acid components of the mixture is obtained. It could be desired to continue cycling until no further improvement of binding is achieved. The number of cycles to be carried out is preferably below 100, more preferably below 10. According to one way of performing the invention, the number of cycles is 7. According to another way of performing the invention, the number of cycles is less than 7, preferentially equal to 6, 5, 4, 3, 2 or 1 cycle(s).

Accordingly, in some embodiments, the combinatorial random library for the SELEX consists of nucleic acid molecules having an internal variable region, (e.g. 10-60 nucleotides), a region comprising a sequence as described herein that is able to form a kissing complex wherein the two region are flanked at the 5′ and 3′ end with primer regions. The primer regions serve as primer binding sites for the amplification step of the SELEX.

In some embodiments, the combinatorial random library for the SELEX consists of nucleic acid molecules having an internal region comprising a sequence NSK1 or NSK2 as above described that is able to form a kissing complex which is flanked by at least one variable region, (e.g. 6-60 nucleotides).

In a particular embodiment, NSKn is a DNA or RNA nucleic acid sequence.

Accordingly a further aspect of the invention relates to a library comprising a plurality of nucleic acid molecules having the general formula 5′-P1-V-NSKₙ-P2-3′ or 5′-P1-NSKₙ-V-P2-3′ wherein P1 and P2 represent the primer regions, V represents the variable region of at least 2 nucleotides, NSKₙ represent the nucleic acid molecule NSK1 or NSK2 as above described.

The primer regions serve as primer binding sites for the amplification step of the SELEX.

In some embodiments, the variable region V comprises 2; 3; 4; 5; 6; 7; 8; 9; 10; 11; 12; 13; 14; 15; 16; 17; 18; 19; 20; 21; 22; 23; 24; 25; 26; 27; 28; 29; or 30 nucleotides.

Accordingly a further aspect of the invention relates to a library comprising a plurality of nucleic acid molecules having the general formula 5′-P1-V1-NSKₙ-V2-P2-3′ wherein P1 and P2 represent the primer regions, V1 and V2 represent the variable region of at least 5 nucleotides, NSKₙ represent the nucleic acid molecule NSK1 or NSK2 as above described.

In some embodiments, each of the variable regions V1 and V2 comprise 2; 3; 4; 5; 6; 7; 8; 9; 10; 11; 12; 13; 14; 15; 16; 17; 18; 19; 20; 21; 22; 23; 24; 25; 26; 27; 28; 29; 30 or more nucleotides.

In some embodiments, the variable regions V1 and V2 have or have not the same length (i.e. the same number of nucleotides).

Accordingly a further aspect of the invention relates to a library comprising a plurality of nucleic acid molecules having the general formula 5′P1-Xn-V1-NSKn-V2-Yn-P2 wherein P1 and P2 represent the primer regions, V1 and V2 represents the variable region of at least 5 nucleotides, Xn and Yn represent a nucleotide sequence of 1, 2, 3 or more nucleotides and Xn and Yn can hybridize, and NSKₙ represent the nucleic acid molecule NSK1 or NSK2 as above described.

In some embodiments, the variable regions V1 and V2 comprise 2; 3; 4; 5; 6; 7; 8; 9; 10; 11; 12; 13; 14; 15; 16; 17; 18; 19; 20; 21; 22; 23; 24; 25; 26; 27; 28; 29; 30 or more nucleotides.

In some embodiments, the variable regions V1 and V2 have or do not have the same length (i.e. the same number of nucleotides).

In some embodiments, Xn represent a nucleotide sequence of 1; 2; 3; 4; 5; 6; 7; 8; 9; 10; 11; 12; 13; 14; 15; 16; 17; 18; 19; 20; 21; 22; 23; 24; 25; 26; 27; 28; 29; 30 or more nucleotides.

In some embodiments, Yn represent a nucleotide sequence of 1; 2; 3; 4; 5; 6; 7; 8; 9; 10; 11; 12; 13; 14; 15; 16; 17; 18; 19; 20; 21; 22; 23; 24; 25; 26; 27; 28; 29; 30 or more nucleotides.

In some embodiments, the kit-of-parts according to the invention comprises at least one nucleic acid molecule NA1 and/or NA2 which is (are) chemically modified.

For example, one potential problem encountered in the use of nucleic acid molecules is that oligonucleotides in their phosphodiester form may be quickly degraded in biological fluids (e.g. body fluids) by intracellular and extracellular enzymes such as endonucleases and exonucleases before the desired effect is manifest.

Examples of such modifications include chemical substitutions at the sugar and/or phosphate and/or base positions. For example U.S. Pat. No. 5,660,985 describes oligonucleotides containing nucleotide derivatives chemically modified at the 2′ position of ribose, 5 position of pyrimidines, and 8 position of purines. U.S. Pat. No. 5,756,703 describes oligonucleotides containing various 2′-modified pyrimidines, and U.S. Pat. No. 5,580,737 describes highly specific nucleic acid ligands containing one or more nucleotides modified with 2′-amino (2′-NH.sub.2), 2′-fluoro (2′-F), and/or 2′-OMe substituents. Techniques for 2′-chemical modification of nucleic acids are also described in the US patent applications No US 2005/0037394 and No US 2006/0264369. Modifications of the nucleic acid molecules contemplated in this invention include, but are not limited to, those which provide other chemical groups that incorporate additional charge, polarizability, hydrophobicity, photosensitivity, hydrogen bonding, electrostatic interaction, staking interaction and fluxionality to the bases or to the nucleic acid molecules as a whole. Modifications to generate oligonucleotide populations which are resistant to nucleases can also include one or more substituted intemucleotide linkages, altered sugars, altered bases, or combinations thereof. Such modifications include, but are not limited to, 2′-position sugar modifications, 5-position pyrimidine modifications, 8-position purine modifications, modifications at exocyclic amines, substitution by 4-thiouridine, substitution by 5-bromo or 5-iodo-uracil, backbone modifications, phosphorothioate or alkyl phosphate modifications, methylations, use of extended aromatic rings and unusual base-pairing combinations such as the isobases isocytidine and isoguanidine. Modifications can also include 3′ and 5′ modifications such as capping.

In some embodiments, the nucleic acid molecules (NA1 and/or NA2) are provided in which the P(O)O group is replaced by P(O)S (“thioate”), P(S)S (“dithioate”), P(O)NR₂ (“amidate”), P(O)R, P(O)OR′, CO or CH₂ (“formacetal”) or 3′-amine (—NH—CH₂—CH₂—), wherein each R or R′ is independently H or substituted or unsubstituted alkyl. Linkage groups can be attached to adjacent nucleotides through an —O—, —N—, or —S— linkage. Not all linkages in the oligonucleotide are required to be identical. As used herein, the term phosphorothioate encompasses one or more non-bridging oxygen atoms in a phosphodiester bond replaced by one or more sulfur atoms.

In some embodiments, the nucleic acid molecules (NA1 and/or NA2) comprise modified sugar groups, for example, one or more of the hydroxyl groups is replaced with halogen, aliphatic groups, or functionalized as ethers or amines. In one embodiment, the 2′-position of the furanose residue is substituted by any of an O-methyl, O-alkyl, O-allyl, S-alkyl, S-allyl, or halo group. Methods of synthesis of 2′-modified sugars are described, e.g., in Sproat, et al., Nucl. Acid Res. 19:733-738 (1991); Cotten, et al., Nucl. Acid Res. 19:2629-2635 (1991); and Hobbs, et al, Biochemistry 12:5138-5145 (1973). Other modifications such as locked sugar ring (LNA) are known to one of ordinary skill in the art.

Another way to obtain highly resistant aptamers is the use of L aptamers (L for levogyre, mirror of the natural enantiomer D). This strategy has been developed by Klussmann and Nolte in 1996 against the targets adenosine and arginine.

Nucleic acid molecules of the invention can be produced recombinantly or synthetically by methods that are routine for one of skill in the art. For example, synthetic RNA molecules can be made as described in US Patent Application Publication No.: 20020161219, or U.S. Pat. Nos. 6,469,158, 5,466,586, 5,281,781, or 6,787,305.

In some embodiments, the kit-of-parts according to the invention comprises at least one nucleic acid molecule NA1 and/or NA2 which is (are) labelled. The term “label” is used herein in a broad sense to refer to agents that are capable of providing a detectable signal, either directly or through interaction with one or more additional members of a signal producing system. According to the invention labels are visual, optical, photonic, electronic, acoustic, opto-acoustic, by mass, electro-chemical, electro-optical, spectrometry, enzymatic, or otherwise chemically, biochemically hydrodynamically, electrically or physically detectable. Label can be, for example tailed reporter, marker or adapter molecules. Accordingly, the nucleic acid molecule is labelled with a detectable molecule selected form the group consisting of radioisotopes, fluorescent compounds, bioluminescent compounds, chemiluminescent compounds, metal chelators or enzymes. Examples of labels include, but are not limited to, the following radioisotopes (e.g., 3H, 14C, 35S, 125I, 131I), fluorescent labels (e.g., FITC, rhodamine, lanthanide phosphors), luminescent labels such as luminol; enzymatic labels (e.g., horseradish peroxydase, beta-galactosidase, luciferase, alkaline phosphatase, acetylcholinestease), biotinyl groups (which can be detected by marked avidin, e.g., streptavidin containing a fluorescent marker or enzymatic activity that can be detected by optical or calorimetric methods), predetermined polypeptide epitopes recognized by a secondary reporter {e.g., leucine zipper pair sequences, binding sites for secondary antibodies, metal binding domains, epitope tags).

In some embodiments, the kit-of-parts according to the invention comprises at least one nucleic acid molecule NA1 and/or NA2 which is immobilized in a solid support, in particular to form a microarray.

In some embodiments, the microarray is high density, with a density over about 100, preferably over about 1000, 1500, 2000, 3000, 4000, 5000 and further preferably over about 9000, 10000, 11000, 12000 or 13000 spots per cm², formed by attaching nucleic acid molecule (NA1 or NA2) onto a support surface.

In some embodiments, the microarray comprises a relatively small number of nucleic acid molecule (NA1 or NA2) (e.g., 10 to 50).

Although the characteristics of the substrate or support may vary depending upon the intended use, the shape, material and surface modification of the substrates must be considered. Although it is preferred that the substrate have at least one surface which is substantially planar or flat, it may also include indentations, protuberances, steps, ridges, terraces and the like and may have any geometric form (e.g., cylindrical, conical, spherical, concave surface, convex surface, string, or a combination of any of these). For example the solid support may be, for example, sheets, strips, membranes, films, gels, beads, microparticles and nanoparticles. Suitable substrate materials include, but are not limited to, glasses, ceramics, plastics, metals, alloys, carbon, papers, agarose, silica, quartz, cellulose, polyacrylamide, polyamide, and gelatin, as well as other polymer supports, other solid-material supports, or flexible membrane supports. Polymers that may be used as substrates include, but are not limited to: polystyrene; poly(tetra)fluoroethylene (PTFE); polyvinylidenedifluoride; polycarbonate; polymethylmethacrylate; polyvinylethylene; polyethyleneimine; polyoxymethylene (POM); polyvinylphenol; polylactides; polymethacrylimide (PMI); polyalkenesulfone (PAS); polypropylene; polyethylene; polyhydroxyethylmethacrylate (HEMA); polydimethylsiloxane; polyacrylamide; polyimide; and various block co-polymers. The substrate can also comprise a combination of materials, whether water-permeable or not, in multi-layer configurations.

There are many established methods for immobilizing assay nucleic acid molecules to a solid support. These include, without limitation, nucleic acid molecules which are immobilized through conjugation of biotin and streptavidin. Such biotinylated assay components can be prepared from biotin-NHS (N-hydroxy-succinimide) using techniques known in the art (e.g., biotinylation kit, Pierce Chemicals, Rockford, Ill.), and immobilized in the wells of streptavidin-coated 96 well plates (Pierce Chemical). In certain embodiments, the surfaces with immobilized assay components can be prepared in advance and stored.

### Methods for Detecting Target Molecules in a Sample

A further aspect of the present invention relates to a method for detecting at least one target molecule in a sample comprising the steps consisting of i) providing a kit-of-parts of the invention which comprises a nucleic acid molecule NA1 or NA2 which is an aptamer specific for the target molecule, ii) bringing into contact the sample with the nucleic acid molecules of the kit-of-parts and iii) detecting the formation of the duplexes formed between the 2 nucleic acids NA1 and NA2.

In some embodiments, a plurality of target molecules is detected in the sample. At least 1, 2, 3, 4, 5, 6, 7, 8, 9, 10, 11, 12, 13, 14, 15, 16, 17, 18, 19, 20, 30, 50, or 100 target molecules are detected in the sample. Accordingly, a further aspect of the present invention also relates to a method for detecting a plurality of target molecules in a sample comprising the steps consisting of i) providing a plurality of kit-of-parts of the invention which comprise a nucleic acid molecule NA1 or NA2 which is an aptamer specific for a target molecule, ii) bringing into contact the sample with the nucleic acid molecules of the kits-of-parts and iii) detecting the formation of the duplexes formed by the two nucleic acids.

In some embodiments, the target molecule(s) is (are) small organic molecule(s).

As used herein the “sample” refers to any sample that is liable to contain the target molecule(s). For example, a sample may further be any biological material that have been isolated from individuals, for example, biological tissues and fluids, which include blood, skin, plasma, serum, lymph, urine, cerebrospinal fluid, tears, smears . . . . A sample may also be a sample of water, in particular drinking water, ground water, surface water or wastewater sample. The sample may also be a sample prepared from a material from the environment, a clinical specimen or a food sample.

In some embodiments, the sample comprises an amount of magnesium (i.e. the kissing complexes are magnesium sensitive).

In some embodiments, the nucleic acid molecule which is the aptamer specific for the target molecule is capable to form a complex with the other nucleic acid molecule of the kit only when it binds to the target molecule (i.e. the aptamer that binds the target molecule undergoes conformational changes upon interactions with the target molecule, thus permitting the formation of the hairpin loop that is able to form the kissing complex).

Detection of the complexes formed between the nucleic acid molecules NA1 and the nucleic acid molecules NA2 (via the formation of the kissing complex) may be performed by any method well known in the art.

In some embodiments, detection can be conducted with nucleic acid molecules as solutes in a liquid phase. In such an assay, the complexes (via the formation of the kissing complex) are separated from individual unbound components by any of a number of standard techniques, including but not limited to chromatography, electrophoresis, filtration . . . . For example, standard chromatographic techniques may also be utilized to separate complexed molecules from unbound ones. For example, gel filtration chromatography separates molecules based on size, and through the utilization of an appropriate gel filtration resin in a column format, for example, the relatively larger complex may be separated from the relatively smaller unbound components. Similarly, the relatively different charge properties of the complex as compared to the unbound components may be exploited to differentiate the complex from unbound components, for example through the utilization of ion-exchange chromatography resins. Such resins and chromatographic techniques are well known to one skilled in the art (see, e.g., Heegaard, N. H., 1998, J. Mol. Recognit. Winter 11(1-6):141-8; Hage, D. S., and Tweed, S. A. J Chromatogr B Biomed Sci Appl 1997 Oct. 10; 699(1-2):499-525). Gel or capillary electrophoresis may also be employed to separate complexes from unbound components (see, e.g., Ausubel et al., ed., Current Protocols in Molecular Biology, John Wiley & Sons, New York, 1987-1999). In this technique, complexes of nucleic acid molecules are separated based on size or charge, for example. In order to maintain the binding interaction during the electrophoretic process, non-denaturing gel matrix materials and conditions in the absence of reducing agent are typically preferred.

In some embodiments, the nucleic acid molecule which is not the aptamer specific for the target molecule is immobilized onto a solid support as above described. Indeed, once immobilized onto a solid support, the nucleic acid molecule can be used as a biosensor element capable of binding to the nucleic acid molecule which is the aptamer specific for the target molecule. A biosensor is an analytical device that integrates a biological element (i.e. the nucleic acid molecules NA1 or NA2) on a solid-state surface, enabling a reversible biospecific interaction with the analyte (i.e. target molecule), and a signal transducer. Biosensors combine high analytical specificity with the processing power of modern electronics to achieve highly sensitive detection systems. In general, these biosensors consist of two components: a highly specific recognition element and a transducer that converts the molecular recognition event into a quantifiable signal. Signal transduction can be accomplished by many methods, including fluorescence, interferometry, gravimetry . . . .

In order to conduct assays with the above mentioned approach, the sample is then contacted with the beads or the microarray upon which the nucleic acid molecule which is not the aptamer specific for the target molecule is immobilized. The then non-immobilized nucleic acid molecule of the kit (i.e. the aptamer) is added. After the reaction is complete (the formation of duplexes between the nucleic acid molecules via the formation of the kissing complex), unbound components (irrelevant target molecules, nucleic acid molecule that did not bind to their target molecules . . . ) may be removed (e.g., by washing) under conditions such that any complex formed will remain immobilized onto the microarray. The detection of the complexes anchored to the microarray may be finally accomplished in a number of methods well known in the art and described herein.

In some embodiments, the nucleic acid molecule (i.e. aptamer) which is not immobilized onto the micorarray can be labelled for the purpose of detection and readout of the assay, either directly or indirectly, with detectable labels discussed herein and which are well-known to one skilled in the art.

It is also possible to directly detect the complex formation without further manipulation or labelling of either component (e.g. aptamer), for example by utilizing the technique of fluorescence energy transfer or fluorescence anisotropy (see EXAMPLE) (see, for example, Lakowicz et al., U.S. Pat. No. 5,631,169; Stavrianopoulos, et al., U.S. Pat. No. 4,868,103). A fluorophore label on the first, ‘donor’ molecule is selected such that, upon excitation with incident light of appropriate wavelength, its emitted fluorescent energy will be transferred to a fluorescent label on a second ‘acceptor’ molecule, which in turn is able to fluoresce due to the absorbed energy. Labels are chosen that emit different wavelengths of light, such that the ‘acceptor’ molecule label may be differentiated from that of the ‘donor’. Since the efficiency of energy transfer between the labels is related to the distance separating the molecules, spatial relationships between the molecules can be assessed. In a situation in which binding occurs between the molecules, the fluorescent emission of the ‘acceptor’ molecule label in the assay should be maximal. A FRET binding event can be conveniently measured through standard fluorometric detection means well known in the art (e.g., using a fluorimeter).

In some embodiments, detection of the complex formation can be accomplished by utilizing a technology such as real-time Biomolecular Interaction Analysis (BIA) (see, e.g., Sjolander, S. and Urbaniczky, C., 1991, Anal. Chem. 63:2338-2345 and Szabo et al., 1995, Curr. Opin. Struct. Biol. 5:699-705). As used herein, “BIA” or “surface plasmon resonance” is a technology for studying biospecific interactions in real time, without labeling any of the interactants (e.g., BIAcore). Changes in the mass at the binding surface (indicative of a binding event) result in alterations of the refractive index of light near the surface (the optical phenomenon of surface plasmon resonance (SPR)), resulting in a detectable signal which can be used as an indication of real-time reactions between biological molecules.

In some embodiments, the detection can be accomplished with an optical biosensor such as described by Edwards and Leatherbarrow (Edwards and Leatherbarrow, 1997, Analytical Biochemistry, 246: 1-6) or also by Szabo et al. (Szabo et al., 1995, Curr. Opinion Struct. Biol., 5(5): 699-705). This technique allows the detection of interactions between molecule in real time, without the need of labelled molecules. This technique is based on the surface plasmon resonance (SPR) phenomenon. For this purpose, a light beam is directed towards the side of the surface area of the substrate that does not contain the sample to be tested and is reflected by said surface. The SPR phenomenon causes a decrease in the intensity of the reflected light with a specific combination of angle and wavelength. The formation of the complex of nucleic acids NA1 and NA2 causes a change in the refraction index on the substrate surface, which change is detected as a change in the SPR signal. This technique is fully illustrated in the EXAMPLE herein.

In some embodiments, the detection can be accomplished with means of piezoelectric transducers which are for example QCM sensors (quartz crystal microbalance) that detect a mass change when the complex is formed. A mass change on the surface of the quartz resonator results in a change in the resonant frequency, which can be quantified.

In some embodiments, the detection can be accomplished by capillary electrophoresis that detects by electrophoresis a mass change when the complex is formed.

In some embodiments, the detection can be accomplished by the alpha-screen technology that allows the emission of luminescence when the complex is formed.

The methods of the invention are particularly suitable—but not restricted to—for use in food, water and environmental analyses. The methods of the invention are also particularly suitable for diagnostic purposes. In particular, the methods of the invention are particularly suitable for the detection of small organic molecules, in any media and environments, particularly in water and other liquids, such as in drinking and wastewater samples. Accordingly, the target molecule can be selected from the group consisting of metabolites, drugs, and pollutants. In a particular embodiment, the media or environment is previously treated with a RNAse inhibitor before contacting said media or environment with the nucleic acid molecules, kit-of-parts or combinatorial library of the invention.

### SELEX in Solution

The present invention also relates to a method for identifying an aptamer directed against a target molecule comprising the following steps:

i) contacting the target molecule with a combinatorial random library according to the invention which consists of a plurality of nucleic acid molecules having an internal region comprising a sequence NSK1 or NSK2 as above described which is flanked by at least one variable region

i) contacting the mixture of step i) with a nucleic acid comprising the corresponding NSK1 or NSK2

iii) partitioning the nucleic acids having affinity for the target molecule from the remainder of the library wherein detecting the formation of the complexes formed between the 2 nucleic acids comprising the sequences NSK1 and NSK2 respectively indicates the presence of nucleic acids having affinity for the target molecule.

In some embodiments, the method may further comprise the steps of amplifying the nucleic acid having affinity to yield a candidate enriched mixture of nucleic acids having affinity for the target molecule, optionally reiterating step i) to iii) in a number of times for selecting the aptamers having the strongest affinity for the target molecule and the step of sequencing and producing the aptamers with the strongest affinity.

Indeed, cycles of selection and amplification are repeated until a desired goal is achieved: identifying the aptamer having the strongest affinity for the target molecule. In the most general case, selection/amplification is continued until no significant improvement in binding strength is achieved on repetition of the cycle.

The method relies on the principle that the aptamer having affinity for the target molecule is capable to form a complex with the nucleic acid comprising the corresponding NSK1 or NSK2 only when it binds to the target molecule (i.e. the aptamer that binds the target molecule undergoes conformational changes upon interactions with the target molecule, thus permitting the formation of the hairpin loop that is able to form the kissing complex).

According to the invention the target molecule is not immobilized on a solid support as classically described for the SELEX™ method but is free in a fluid sample. Typically, the fluid sample is an aqueous solution.

As used herein, a “library” is a mixture of nucleic acid molecules, referred to as library “members”, which are potentially capable of binding to the target molecule. Typically, the members of the library are randomised in sequence such that a large number of the possible sequence variations are available within the library. The randomised region(s) may be in essence of any length, but a length of up to 100 nucleotides, which may be interspersed with non-randomised insertion(s), is preferred. Typically, the randomised region will be between 2 and 60 or more.

The randomised portion of the library members can be derived in a number of ways. For example, full or partial sequence randomisation can be readily achieved by direct chemical synthesis of the members (or portions thereof) or by synthesis of a template from which the members (or portions thereof) can be prepared by use of appropriate enzymes. End addition, catalysed by terminal transferase in the presence of non limiting concentrations of all four nucleotide triphosphates can add a randomised sequence to a segment. Sequence variability in the test nucleic acids can also be achieved by employing size-selected fragments of partially digested (or otherwise cleaved) preparations of large, natural nucleic acids, such as genomic DNA preparations or cellular RNA preparations. A randomised sequence is preferably generated by using a mixture of all four nucleotides (preferably in the ratio 6:5:5:4, A:C:G:T, to allow for differences in coupling efficiency) during the synthesis of each nucleotide in that stretch of the oligonucleotide library. However as mentioned above the nuclei acid sequences can comprise modified nucleotides. Examples of such modifications include chemical substitutions at the sugar and/or phosphate and/or base positions as above described (e.g. nucleotide derivatives chemically modified at the 2′ position of ribose, 5 position of pyrimidines, and 8 position of purines) Modifications of the nucleic acid molecules also include, but are not limited to, those which provide other chemical groups that incorporate additional charge, polarizability, hydrophobicity, hydrogen bonding, electrostatic interaction, and fluxionality to the bases or to the nucleic acid molecules as a whole. Modifications to generate oligonucleotide populations which are resistant to nucleases can also include one or more substitute internucleotide linkages, altered sugars, altered bases, or combinations thereof. Such modifications include, but are not limited to, 2′-position sugar modifications, 5-position pyrimidine modifications, 8-position purine modifications, modifications at exocyclic amines, substitution of 4-thiouridine, substitution of 5-bromo or 5-iodo-uracil, backbone modifications, phosphorothioate or alkyl phosphate modifications, methylations, and unusual base-pairing combinations such as the isobases isocytidine and isoguanidine. Modifications can also include 3′ and 5′ modifications such as capping. In some embodiments, the nucleic acid molecules (NA1 and/or NA2) are provided in which the P(O)O group is replaced by P(O)S (“thioate”), P(S)S (“dithioate”), P(O)NR₂ (“amidate”), P(O)R, P(O)OR′, CO or CH₂ (“formacetal”) or 3′-amine (—NH—CH₂—CH₂—), wherein each R or R′ is independently H or substituted or unsubstituted alkyl. Linkage groups can be attached to adjacent nucleotides through an —O—, —N—, or —S— linkage. Not all linkages in the oligonucleotide are required to be identical. As used herein, the term phosphorothioate encompasses one or more non-bridging oxygen atoms in a phosphodiester bond replaced by one or more sulfur atoms. In some embodiments, the nucleic acid molecules comprise modified sugar groups, for example, one or more of the hydroxyl groups is replaced with halogen, aliphatic groups, or functionalized as ethers or amines. In one embodiment, the 2′-position of the furanose residue is substituted by any of an O-methyl, O-alkyl, O-allyl, S-alkyl, S-allyl, or halo group. Methods of synthesis of 2′-modified sugars are described, e.g., in Sproat, et al., Nucl. Acid Res. 19:733-738 (1991); Cotten, et al., Nucl. Acid Res. 19:2629-2635 (1991); and Hobbs, et al, Biochemistry 12:5138-5145 (1973). Other modifications are known to one of ordinary skill in the art. Modifications can also include 3′ and 5′ modifications such as capping. Another way to obtain highly resistant aptamers is the use of artificial L aptamers built from L nucleotides, mirror image of natural nucleotides and resistant to nuclease. This strategy has been developed by Klussmann and Nolte in 1996 against the targets adenosine and arginine Nucleic acid molecules of the invention can be produced recombinantly or synthetically by methods that are routine for one of skill in the art. For example, synthetic RNA molecules can be made as described in US Patent Application Publication No.: 20020161219, or U.S. Pat. Nos. 6,469,158, 5,466,586, 5,281,781, or 6,787,305.

In some embodiments, the library consists of a plurality of nucleic acid molecules having the general formula 5′-P1-V-NSKn-P2-3′ or 5′-P1-NSKn-V-P2-3′ wherein P1 and P2 represent the primer regions, V represents the variable region of at least 2 nucleotides, NSKn represent the nucleic acid molecule NSK1 or NSK2 as above described.

In some embodiments, each of the variable region V comprises 2; 3; 4; 5; 6; 7; 8; 9; 10; 11; 12; 13; 14; 15; 16; 17; 18; 19; 20; 21; 22; 23; 24; 25; 26; 27; 28; 29; 30 or more nucleotides.

In some embodiments, the library consists of a plurality of nucleic acid molecules having the general formula 5′-P1-V1-NSKn-V2-P2-3′ wherein P1 and P2 represent the primer regions, V1 and V2 represent the variable region of at least 5 nucleotides, NSKn represent the nucleic acid molecule NSK1 or NSK2 as above described.

In some embodiments, each of the variable regions V1 and V2 comprise 2; 3; 4; 5; 6; 7; 8; 9; 10; 11; 12; 13; 14; 15; 16; 17; 18; 19; 20; 21; 22; 23; 24; 25; 26; 27; 28; 29; 30 or more nucleotides.

In some embodiments, the library consists of a plurality of nucleic acid molecules having the general formula 5′P1-Xn-V1-NSKn-V2-Yn-P2 wherein P1 and P2 represent the primer regions, V1 and V2 represent the variable region of at least 5 nucleotides, Xn and Yn represent a nucleotide sequence of 1, 2, 3 or more nucleotides and Xn and Yn can hybridize, and NSKn represent the nucleic acid molecule NSK1 or NSK2 as above described.

In some embodiments, each of the variable regions V1 and V2 comprise 2; 3; 4; 5; 6; 7; 8; 9; 10; 11; 12; 13; 14; 15; 16; 17; 18; 19; 20; 21; 22; 23; 24; 25; 26; 27; 28; 29; 30 or more nucleotides.

In some embodiments, Xn represents a nucleotide sequence of 1; 2; 3; 4; 5; 6; 7; 8; 9; 10; 11; 12; 13; 14; 15; 16; 17; 18; 19; 20; 21; 22; 23; 24; 25; 26; 27; 28; 29; 30 or more nucleotides.

In some embodiments, Yn represents a nucleotide sequence of 1; 2; 3; 4; 5; 6; 7; 8; 9; 10; 11; 12; 13; 14; 15; 16; 17; 18; 19; 20; 21; 22; 23; 24; 25; 26; 27; 28; 29; 30 or more nucleotides.

In some embodiments, therefore, the present invention relies on the establishment of equilibrium for a substantial number of the library members, especially those having slow dissociation kinetics. Preferably, the library and the target molecule are incubated together for a sufficient time to allow interaction between the target molecules and the members of the library especially for a sufficient time that will allow the conformational rearrangement of the members of the library with the target molecules. The period required will depend on the target and library, and also on the round of selection; preferably, for example, the first round of selection may involve an incubation of between about 5 min (or less) and about 48 hours. Advantageously, the first round of selection is at least about 30 minutes to about 4 hours, preferably 1 hour. The remaining rounds involve an incubation of at least 30 minutes to about 4 hours, preferably 1 hour, in order to allow the establishment of a full equilibrium.

In some embodiments, the corresponding nucleic acid molecule is immobilized on a solid support as above described.

In some embodiments, the corresponding nucleic acid molecule is free in solution.

In some embodiments, the method further comprises a step consisting of a counter-selection of the library, in absence of the target molecule, against the immobilized hairpin (otherwise referred to as “aptakiss” in the present application) and the support in order to eliminate the non specific candidates and candidates that could form a kissing complex with the immobilized hairpin without the target molecule.

In some embodiments, the method further comprises a step of collecting the positive candidates. Typically, as the kissing complexes are magnesium sensitive, the elution of the positive candidates will be carried out with EDTA (ethylenediaminetetraacetic acid). Any other methods used in classical SELEX methods for elution of the positive candidates could be performed. New methods could be considered for the specific elution of the positive candidates: i) The immobilized hairpins would be a DNA-RNA chimeric molecule showing at the bottom of the stem a DNA enzyme restriction site. The enzymatic digestion would allow the elution of the complexe (Aptamer-target-immobilized hairpin) avoiding the elution of the non specific candidates ii) In the same way, the immobilized hairpin would be a DNA-RNA chimeric molecule consisting of a DNA strand (NS1) and a RNA complementary strand (NS2) in the stem. The elution step could be done by enzymatic digestion with the RNase H that recognizes the DNA-RNA duplexes.

Any method as above described may be used for the detection of the complexes formed between the nucleic acid molecules NA1 and NA2 (e.g. chromatography, electrophoresis, filtration, FRET, surface plasmon resonance, luminescence . . . ).

Typically target molecules can be—but are not restricted to—small organic or inorganic molecules, carbohydrates, nucleic acid molecule and derivatives, lipids, microorganisms, viruses, amino acids, antibiotics, peptides, polypeptides, proteins, polymers, macromolecules, complex targets, etc. as above defined.

The invention will be further illustrated by the following figures and examples. However, these examples and figures should not be interpreted in any way as limiting the scope of the present invention.

### FIGURES

FIG. 1. Secondary structures of aptakiss and aptaswitches used in this study. The sequence of the different oligonucleotide derivatives used is given in the Table S 1. Deoxyribonucleotides are indicated in blue and ribonucleotides in black except those that engage loop-loop interaction shown in red. Point mutations in the aptakiss/adenoswitch loop appear in black.

FIG. 2. SPR analysis of GTPswitch/aptakiss complex. GTPswitch (20 microM in 10 mM K₂HPO₄ pH 6.2 containing 200 mM KCl and 10 mM MgCl₂) was injected over a chip on which biotinylated aptakiss was immobilized in the presence of increasing concentrations (from 0 to 1 mM) of either GTP (top left) or ATP (bottom left). The maximum SPR signal obtained was plotted as a function of nucleotide triphosphate concentration (top right); results are expressed as a mean±sem of two individuals experiments. As a control GTPswitch in the presence of GTP (from 0 to 1 mM) was injected over a chip functionalized with biotinylated aptakissmut (bottom right).

FIG. 3. SPR sensorgrams of adenoswitch/aptakiss complex against immobilized biotinylated aptakiss. Upper: adenoswitch (5 microM in 10 mM Tris pH 7.4 containing 100 mM NaCl and 10 mM MgCl₂) was injected in the presence of increasing amounts (0, 0.125, 0.25, 0.5, 1, 2, 4, 8 mM) of adenosine. Lower: similar experiment under same conditions at 8 mM adenosine with increasing concentrations of adenoswitch (0, 0.08, 0.16 0.31, 0.63, 1.25, 2.5 or 5 microM).

FIG. 4. SPR sensorgrams of three adenoswitch variants (0.625 microM) were injected (in 10 mM Tris, 100 mM NaCl, 10 mM MgCl₂) in the presence of 8 mM adenosine against immobilized biotinylated apatkiss (upper panel). SPR sensorgrams for the adenoswitchATGC in the presence of increasing amounts of adenosine (0, 0.13, 0.25, 0.5, 1, 2, 4 or 8 mM) (lower panel).

FIG. 5. Homogeneous fluorescence anisotropy assay of aptakiss-adenoswitch complexes (binding buffer: 10 mM Tris, pH 7.5, 100 mM NaCl, 10 mM MgCl2; reaction temperature: 4° C.). Dose response curves were obtained using 10 nM aptakiss-TR and 10 nM adenoswitch (black triangle, adenosine). 10 nM adenoswitchTAGC (black diamonds, adenosine; open square, inosine) or 10 nM adenoswitchTAGCmut2 (open diamonds, adenosine). Δr=r−r0 where r0 is the fluorescence anisotropy in the absence of ligand. Triplicate experiments.

FIG. 6. SPR analysis of Kx1 to Kx4 hairpins.

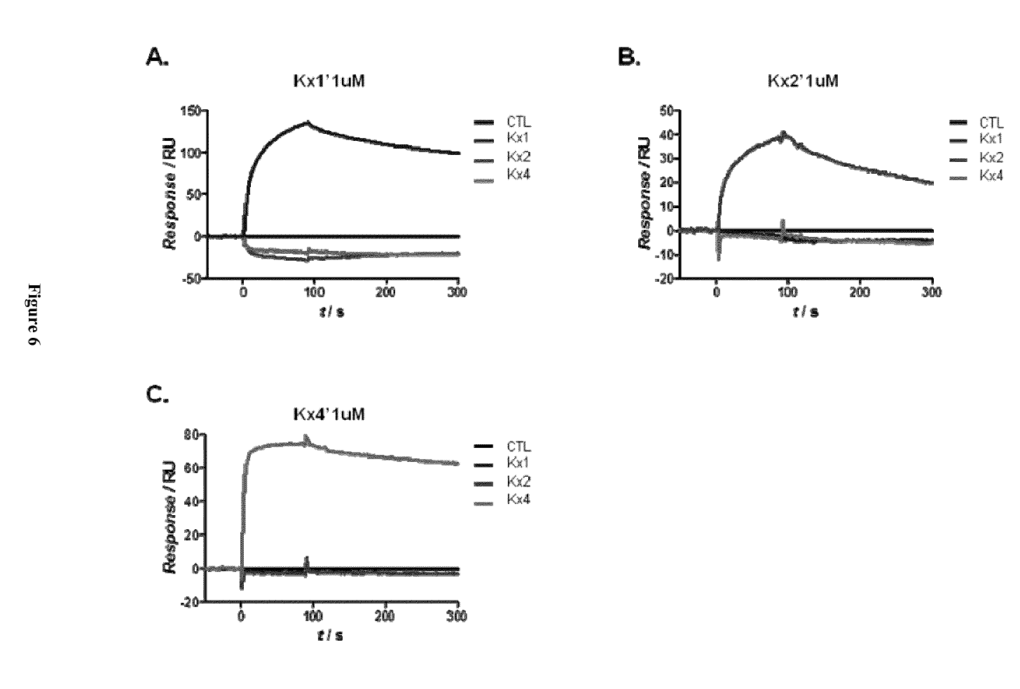

FIG. 7. Melting transition of different complexes.

FIG. 8. Sequence and structure of anti-adenosine, ADOsw1′, anti-GTP, GTPsw2′, anti-theophylin, THEsw4′ aptamers.

FIG. 9. SPR analysis of GTPsw2′-GTP and THEsw4′-theophyllin complexes by immobilized Kx2 or Kx4.

FIG. 10. SPR analysis of aptaswitch-ligand mixtures on 4 channel SPR chip.

FIG. 11. a) Schematic representation of the apical loop of the selected DNA aptamer DII21 against the RNA hairpin TAR of HIV-1. b) Adenoswitches D1121 models A, B and C with the DNA loop of the aptamer DII21, three connectors of varying size (3, 2 and 1 base pair) combining the DNA DII21 loop with the part of the DNA aptamer that bind the adenosine.

FIG. 12. Comparison by using fluorescence anisotropy of the capacity of these three DII21A, DII21B, and DII21C models to bind TAR.

FIG. 13. Schematic representation of the libraries used for the “DNA SELKISS.” a) Degenerated sequences are in the connector located between the DII21 loop and the adenosine aptamer binding region. b) Degenerated sequences are located in the region of the aptamer responsible for the binding to adenosine.

### EXAMPLE 1

### Summary

Kissing complexes are formed by RNA stem loops interacting to each other through the loops. These complexes are involved in numerous biological processes such as the control of the DNA replication of plasmids or the dimerization of the genomic RNA of virus. Moreover, RNA hairpins have been targeted by <<in vitro>> selection and RNA hairpin aptamers have been identified. It has been shown that the interacting loops generated kissing complexes. Studies of these loop-loop interactions have been well documented but in order to investigate if some rules could guide their formation, specificity and stability, we have performed an <<in vitro>> selection of RNA hairpins for their capacity to kiss. Some loop-loop complexes of high affinity have been identified. By sequence analysis of the <<in vitro>> selection results, we have characterized new RNA motifs in the double helix forming by the interaction of the loops. These works allowed to obtain a catalog of RNA hairpins able to interact via their loops with high affinity to form kissing complexes. We named these nucleic acid molecules: Aptakiss.

Materials and Methods:

Oligonucleotides

RNA random libraries used for selection I and II, containing 10 or 11 random nucleotides or a consensus motif flanked by invariant primer annealing sites:

| | |
|---|---|
| A | UUACCAGCCUUCACUGCUCG- |
| | N10/11-CGAGCACCACGGUCGGUCACAC |
| B | 5′ACGAGC-NRNGGN-GCUCGU biotin |
| C | 5′GGUUACCAGCCUUCACUGCUCG- |
| | NCCNYN- |
| | CGAGCACCACGGUCGGUCACAC |
| D | 5′GGGAGGACGAAGCGGACGAGC- |
| | NRNGGN- |
| | GCUCGUCAGAAGACACGCCCGA |

and various RNA aptamers were chemically synthesized on an Expedite 8909 synthesizer (Applied Biosystems). The stem sequences are underlined. Two different primers (Proligo): P20 5′GTGTGACCGACCGTGGTGC complementary to the 3′ end of the libraries A and C and 3′SL, same polarity as the RNA pool and containing the T7 transcription promoter (underlined)

5′TAATACGACTCACTATAGGTTACCAGCCTTCACTGC

were used for PCR amplification. Primers P1A

5′TAATACGACTCACTATAGGGAGGACGAAGCGG

and P2A 5′TCGGGCGTGTCTTCTG were used for handle library D. All oligonucleotides and transcription products were purified by electrophoresis on denaturing 20% polyacrylamide, 7M urea gels.

In Vitro Selection I:

The labeled RNA library A (50 picomoles) with [γ 32-P]ATP (10 mCi/mL) (4500 Ci/mmol) from ICN Pharmaceutical, was mixed at room temperature in a final volume of 10 μL of the R buffer (20 mM HEPES, 20 mM sodium acetate, 140 mM potassium acetate, and 3 mM magnesium acetate, pH 7.4) for 24 h. In the first round of selection, the stringency was low enough to retain in the selected pool the sequences able to kiss. In subsequent rounds of selection, to keep only high stability complexes, the RNA hairpin concentration was decreased 10 times at each round. Moreover, the time of incubation was decreased (24 h for round 1, 6 h for round 2, 1 h for round 3 and 10 min for the final round). RNA population was separated by Electrophoretic Mobility Shift Assay, EMSA. Samples were runned on a native gel (15% [w/v], 75:1 acrylamide:bis-acrylamide) in 50 mM Tris-acetate (pH 7.3 at 20° C.) and 3 mM magnesium acetate (TAC buffer) at 100 V and 4° C. for 15 h. The bands were visualized and quantified by Instant Imager (Packard Instrument). The bands corresponding to the RNA shifted complexes were extracted from the gel, eluted for 16 h at 4° C., in 600 μl of the elution buffer (10 mM Tris-HCl, pH 7.4, 1 mM ethylenediaminetetraacetic acid (EDTA), and 25 mM NaCl.), and then, ethanol precipitated.

RNA Amplification, Cloning, and Sequencing:

Extracted RNA hairpins were denatured at 95° C. for 40 sec and placed on ice for 2 min. Then, RNA pool was copied into cDNA using 5 units of the EZrTth (Perkin elmer) polymerase at 63° C. for 30 min according to the manufacturer's protocol. The candidates were amplified in the same tube containing the EZrTth buffer in addition to 300 μM of dNTP, 25 mM of MnOAc and 2 μM of each primer. Then, the reaction mixture was denatured to 94° C. for 2 min and was subjected to repeated cycles: 94° C. for 1 min, 63° C. for 1 min, for 40 cycles and 63° C. for 7 min, for one final cycle. RNA hairpins were obtained by in vitro transcription, after precipitation of the PCR products with the Ampliscribe T7 high yield transcription kit from TEBU including [α32-P]UTP (10 mCi/mL) (4500 Ci/mmol) from ICN Pharmaceutical. The transcription products were purified by electrophoresis on 20% denaturing polyacrylamide gels and then used for the next selection cycle. After 4 cycles, selected sequences were cloned using the TOPO TA cloning kit from Invitrogen and sequenced by using the dRhodamine Terminator Cycle sequencing kit from Perkin-Elmer, according to the manufacturers' instructions.

In Vitro Selection II a:

The biotinylated RNA library B was mixed for 1 hour at room temperature in the R buffer with library A (CCNY) at 50 nM (FIG. 14). Prior to use, library A was submitted to a counter-selection. Library A was mixed with streptavidin beads (20 μg of Streptavidin MagneSphere Paramagnetic Particles from Promega) previously equilibrated in R buffer and RNA candidates non retained by the beads were used for selection II. RNA complexes formed with library B, containing a biotin, and library A were captured with streptavidin beads for 10 min Unbound RNA was removed, and the beads were washed with 100 μl of R buffer. The bound candidates of library A were eluted from the library B by heating for 45 s at 85° C. in 50 μl of water. RNA candidates were submitted to RT-PCR and transcription as described for selection I. A second round of selection with 5 nM of libraries A and B was added. Sequences from the two rounds of selection were cloned as described above. These sequences were classified in five different families according to consensus nucleotide sequences at the stem-loop junction. Members of family 1 had got a GG closing base pair, family 2 a AG, family 3 a GU or UG, family 4 and family 5 all other sequences of the first or second round of selection, respectively.

In Vitro Selection II b

Amplification of these candidates were performed with a new primer P20 containing a poly-T tail at the 5′ end. So, PCR products were in vitro transcribed into poly-A tailed RNA candidates. Poly-A candidates were immobilised on streptavidin beads by hybridization to a biotinylated complementary poly-T oligonucleotide. A new round of selection at 50 nM for families 1, 2, 3, 4 and 5 nM for family 5 were performed against these candidates with the D library to identify NRNGGN partners of NCCNYN candidates. Primers of the D library have been changed compared to library A. This change was important because it allowed to amplify only D candidates and not C known candidates. The selection protocol was the same as the first round of selection IIa with the A library excepted that the counter-selection has been carried out against a mixture of Poly-T-biotinylated primer alone and Poly-T-biotinylated primer hybridized with a RNA poly-A oligonucleotide on streptavidin beads.

Electrophoretic Mobility Shift Assay (EMSA)

Dissociation constant (Kd) of loop-loop RNA complexes was determined using electrophoretic mobility shift assay. In general, 0.1 or 1 nM of 32P 5′ end-labeled hairpin was incubated with increasing concentrations of partners for 20 min at 23° C. in 10 μl of R buffer. Binding reactions were loaded onto non denaturing native gels [12% (wt/v) 19:1 acrylamide/bis(acrylamide) in 50 mM Tris-acetate (pH7.3 at 20° C.) and 3 mM magnesium acetate] equilibrated at 4° C. and electrophoresed overnight at 120 V (6V/cm). Complexes were quantified by Instant Imager analysis (Hewlett-Packard). Kd values were deduced from data point fitting with Kaleidagraph 3.0 (Abelbeck software), according to the equation: B=(Bmax)([L]0)/([L]0+Kd), where B is the proportion of complex, Bmax the maximum of complex formed and [L]0 the total of unlabeled ligand.

Thermal Denaturation of RNA Complexes

RNA hairpins and complexes were prepared in 20 mM sodium cacodylate buffer, pH 7.3 at 20° C., containing 140 mM potassium chloride, 20 mM sodium chloride and 0,3; 3 or 10 mM magnesium chloride. RNA samples were prepared at 1 μM final concentration. Samples were denatured at 90° C. for 1 min and 30 sec and placed on ice for 10 min After an incubation of 10 min at room temperature, RNA sequences were mixed and incubated for 30 min. Denaturation of the samples was achieved by increasing the temperature at 0.4° C./min from 4 to 90° C. and was followed at 260 nm Thermal denaturation was monitored in a Cary 1 spectrophotometer interfaced with a Peltier effect device that controls temperature within +0.1° C.

Surface Plasmon Resonance Kinetic Measurements

SPR experiments were performed on a BIAcore 2000 or 3000 apparatus (Biacore AB, Sweden) running with the BIAcore 2.1 software. Biotinylated hairpin RNA (150-1000 RU), was immobilized at 50 nM at a flow rate of 5 μl/min on SA sensorchips in the R selection buffer according to the procedure described in the BIA applications handbook. One streptavidin-coated flow-cell was used to check for nonspecific binding of RNA hairpins. The signals from these control channels served as base lines and were subtracted to the RU change observed when complexes were formed. The sensorship surface was successfully regenerated with one 20-μl pulse of 3 mM EDTA, followed by one 20-μl pulse of distilled water and finally one 20 μl pulse of R buffer. Nonlinear regression analysis of single sensorgrams at five concentrations, at least, of injected RNAs at 23° C. was used to determine the kinetic parameters of the complex formation. The data were analyzed with the BIA evaluation 2.2.4 software, assuming a pseudo-first order model, according to Equations 1-2, for the association and dissociation phases, respectively, where R is the signal response, Rmax the maximum response level, C the molar concentration of the injected RNA molecule, kon the association rate constant, and koff the dissociation rate constant.

Results:

Selex I

Kissing complexes are formed by RNA stem loops interacting to each other through the loops. These complexes are involved in numerous biological processes such as the control of the DNA replication of plasmids or the dimerization of the genomic RNA of virus. Moreover, RNA hairpins have been targeted by <<in vitro>> selection and RNA hairpin aptamers have been identified. It has been shown that the interacting loops generated kissing complexes. Studies of these loop-loop interactions have been well documented but in order to investigate if some rules could guide their formation, specificity and stability, we have performed an <<in vitro>> selection of RNA hairpins for their capacity to kiss.

RNA random libraries used for selection I, contained 10 or 11 random nucleotides flanked by invariant primer annealing sites.

Some loop-loop complexes of high affinity have been identified. By sequence analysis of the <<in vitro>> selection results, we have characterized new RNA motifs in the double helix formed by the interaction of the loops. These works allowed to obtain a catalog of RNA hairpins able to interact via their loops with high affinity to form kissing complexes.

RNA population was separated by Electrophoretic Mobility Shift Assay, EMSA. Samples were runned on a native gel (15% [w/v], 75:1 acrylamide:bis-acrylamide) in 50 mM Tris-acetate (pH 7.3 at 20° C.) and 3 mM magnesium acetate (TAC buffer) at 100 V and 4° C. for 15 h. The bands were visualized and quantified by Instant Imager (Packard Instrument). The bands corresponding to the RNA shifted complexes were extracted from the gel, eluted for 16 h at 4° C., in 600 μl of the elution buffer (10 mM Tris-HCl, pH 7.4, 1 mM ethylenediaminetetraacetic acid (EDTA), and 25 mM NaCl.), and then, ethanol precipitated.

Extracted RNA hairpins were denatured at 95° C. for 40 sec and placed on ice for 2 min. Then, RNA pool was copied into cDNA using 5 units of the EZrTth (Perkin elmer) polymerase at 63° C. for 30 mM according to the manufacturer's protocol. The candidates were amplified in the same tube containing the EZrTth buffer in addition to 300 μM of dNTP, 25 mM of MnOAc and 2 μM of each primer. Then, the reaction mixture was denatured to 94° C. for 2 min and was subjected to repeated cycles: 94° C. for 1 min, 63° C. for 1 min, for 40 cycles and 63° C. for 7 min, for one final cycle. RNA hairpins were obtained by in vitro transcription, after precipitation of the PCR products with the Ampliscribe T7 high yield transcription kit from TEBU including [α32-P]UTP (10 mCi/mL) (4500 Ci/mmol) from ICN Pharmaceutical. The transcription products were purified by electrophoresis on 20% denaturing polyacrylamide gels and then used for the next selection cycle. After 4 cycles, selected sequences were cloned using the TOPO TA cloning kit from Invitrogen and sequenced by using the dRhodamine Terminator Cycle sequencing kit from Perkin-Elmer, according to the manufacturers' instructions.

110 RNA candidates were analysed. Consensus and complementary sequences were searched. For example: Sequences were classified in 71 families, each family containing at least 3 sequences able to match one presumed complementary sequence through four contiguous base pairing:

Sequences of the loops with at least 3 partners and alignments:

K1 -> K11:K14:K15:K1:K28:K34:K39:K51:K52:K53:K59:K61:K73:K75:K85:

| | |
|---|---|
| K1 | ATGTGCGCCAA |
| K11 | TGGACGCCTTC |
| K14 | AGGCGCGCAATG |
| K15 | CNGGTGGTTA |
| K28 | TCTTCGCGTT |
| K34 | TACAGGAATA |
| K39 | ACGGGGTTCT |
| K51 | TGGTTTTTACG |
| K52 | AAGGCGGAG |
| K53 | TACACGGTCT |
| K59 | GATTCGGTGT |
| K61 | TGTCCCGCGTT |
| K73 | AGGTTAGCGA |
| K75 | TTTGGTTTCT |
| K85 | ACACTG |

K10 -> K13:K25:K29:K37:K4:K60bis:K61:K74:K86:K94:

| | |
|---|---|
| K10 | ACAGCTCAGAA |
| K13 | TGAGTCGCA |
| K25 | GCCTGTCTAA |
| K29 | AGTCTGAATG |
| K37 | TGTCCGCTGT |
| K4 | TAGTGAGTTTT |
| K60bis | TGTCATCTCCc |
| K61 | TGTCCCGCGTT |
| K74 | TCCGGAGTTT |
| K86 | TTTGTCCTTC |
| K94 | GTCGTTCTCG |

K11 -> K14:K17:K1:K30:K37:K44:K52:K61:K78:K82:K86:K87:K98:

| | |
|---|---|
| K11 | CTTCCGCAGGT |
| K14 | AGGCGCGCAATG |
| K17 | ACGTCCCCT |
| K1 | AACCGCGTGTA |
| K30 | GTCCGGGTTA |
| K37 | TGTCCGCTGT |
| K44 | ACAGGGCGTCT |
| K52 | AAGGCGGAG |
| K61 | TGTCCCGCGTT |
| K78 | ATCGGCGTTT |
| K82 | GGAAGGTGAG |
| K86 | TTTGTCCTTC |
| K87 | ATCGGCGTTT |
| K98 | TCCATGCGCA |

K13 -> K10:K18:K24:K35:K36:K50:K73:K96:K98:

| | |
|---|---|
| K13 | ACGCTGAGT |
| K10 | AAGACTCGACA |
| K18 | TCCTGCGCA |
| K24 | TGCGCCGATC |
| K35 | GCGAAATG |
| K36 | ATTACTTGCG |
| K50 | GACTTCCGCA |
| K73 | AGGTTAGCGA |
| K96 | CGACGTGACA |
| K98 | TCCATGCGCA |

K14 -> K11:K14:K18:K21:K24:K2:K32:K61:K70:K97:K98:

| | |
|---|---|
| K14 | GTAACGCGCGGA |
| K11 | TGGACGCCTTC |
| K18 | TCCTGCGCA |
| K21 | GCCTCCCCTTT |
| K24 | TGCGCCGATC |
| K2 | GCGCCAGTAG |
| K32 | TGCCTCCGTT |
| K61 | TGTCCCGCGTT |
| K70 | CTAGGAGCCTGG |
| K97 | TTGCAGTGGG |
| K98 | TCCATGCGCA |

K15 -> K1:K76:K89:

| | |
|---|---|
| K15 | ATTGGTGGNC |
| K1 | AACCGCGTGTA |
| K76 | GTCACCTGTTA |
| K89 | AGTATTAACG |

K16 -> K24:K30:K36:K39:K52:K63:K90:K98:

| | |
|---|---|
| K16 | GTAACGCCCA |
| K24 | TGCGCCGATC |
| K30 | GTCCGGGTTA |
| K36 | ATTACTTGCG |
| K39 | ACGGGGTTCT |
| K52 | AAGGCGGAG |
| K63 | ATGATGGGTG |
| K90 | TGGGTGGTAAT |
| K98 | TCCATGCGCA |

K17 -> K11:K17:K20:K38:K39:K8:K96:

| | |
|---|---|
| K17 | TCCCCTGCA |
| K11 | TGGACGCCTTC |
| K20 | TGGGATGGGTG |
| K38 | TCACTTGACG |
| K39 | ACGGGGTTCT |
| K8 | AGGGGGGATAG |
| K96 | CGACGTGACA |

K18 -> K14:K16:K18:K24:K2:K34:K36:K41:K44:K50:K58:K5:K70:K97:K98:

| | |
|---|---|
| K18 | ACGCGTCCT |
| K14 | AGGCGCGCAATG |
| K16 | ACCCGCAATG |
| K24 | TGCGCCGATC |
| K2 | GCGCCAGTAG |
| K34 | TACAGGAATA |
| K36 | ATTACTTGCG |
| K41 | TCGGCAGGTAT |
| K44 | ACAGGGCGTCT |
| K50 | GACTTCCGCA |
| K58 | AGGTGGAGGA |
| K5 | TCGCACCCTCA |
| K70 | CTAGGAGCCTGG |
| K97 | TTGCAGTGGG |
| K98 | TCCATGCGCA |

K2 -> K14:K18:K24:K2:K36:K45:K52:K78:K87:K98:

| | |
|---|---|
| K2 | GATGACCGCG |
| K14 | AGGCGCGCAATG |
| K18 | TCCTGCGCA |
| K24 | TGCGCCGATC |
| K36 | ATTACTTGCG |
| K45 | GAGCTGGTGTT |
| K52 | AAGGCGGAG |
| K78 | ATCGGCGTTT |
| K87 | ATCGGCGTTT |
| K98 | TCCATGCGCA |

K20 -> K16:K17:K5:K60bis:K61:K76:K98:

| | |
|---|---|
| K20 | GTGGGTAGGGT |
| K16 | ACCCGCAATG |
| K17 | ACGTCCCCT |
| K5 | TCGCACCCTCA |
| K60bis | TGTCATCTCCc |
| K61 | TGTCCCGCGTT |
| K76 | GTCACCTGTTA |
| K98 | TCCATGCGCA |

K21 -> K39:K44:K52:K58:K64:K82:K8:

| | |
|---|---|
| K21 | TTTCCCCTCCG |
| K39 | ACGGGGTTCT |
| K44 | ACAGGGCGTCT |
| K52 | AAGGCGGAG |
| K58 | AGGTGGAGGA |
| K64 | GGGGTGAGAG |
| K82 | GGAAGGTGAG |
| K8 | AGGGGGGATAG |

K24 -> K14:K18:K24:K2:K41:K42:K50:K52:K5:K77:K78:K87:K92bis:

| | |
|---|---|
| K24 | CTAGCCGCGT |
| K14 | AGGCGCGCAATG |
| K18 | TCCTGCGCA |
| K2 | GCGCCAGTAG |
| K41 | TCGGCAGGTAT |
| K42 | TTTATCGCTTTT |
| K50 | GACTTCCGCA |
| K52 | AAGGCGGAG |
| K5 | TCGCACCCTCA |
| K77 | ACGGCCAGA |
| K78 | ATCGGCGTTT |
| K87 | ATCGGCGTTT |
| K92bis | ACGGCTG |

K25 -> K10:K34:K41:K44:

| | |
|---|---|
| K25 | AATCTGTCCG |
| K10 | AAGACTCGACA |
| K34 | TACAGGAATA |
| K41 | TCGGCAGGTAT |
| K44 | ACAGGGCGTCT |

K27 -> K27:K2:K32:K5:K76:K80:K91:K98:

| | |
|---|---|
| K27 | GTACGGTGGA |
| K2 | GCGCCAGTAG |
| K32 | TGCCTCCGTT |
| K5 | TCGCACCCTCA |
| K76 | GTCACCTGTTA |
| K80 | TAGTGCCGTA |
| K91 | GGGCATGTTA |
| K98 | TCCATGCGCA |

K28 -> K11:K14:K1:K28:K35:K61:K89:

| | |
|---|---|
| K28 | TTGCGCTTCT |
| K11 | TGGACGCCTTC |
| K14 | AGGCGCGCAATG |
| K1 | AACCGCGTGTA |
| K35 | GCGAAATG |
| K61 | TGTCCCGCGTT |
| K89 | AGTATTAACG |

K29 -> K10:K50:K59:K77:

| | |
|---|---|
| K29 | GTAAGTCTGA |
| K10 | AAGACTCGACA |
| K50 | GACTTCCGCA |
| K59 | GATTCGGTGT |
| K77 | ACGGCCAGA |

K30 -> K11:K16:K1:K30:K52:K61:K74:K89:

| | |
|---|---|
| K30 | ATTGGGCCTG |
| K11 | TGGACGCCTTC |
| K16 | ACCCGCAATG |
| K1 | AACCGCGTGTA |
| K52 | AAGGCGGAG |
| K61 | TGTCCCGCGTT |
| K74 | TCCGGAGTTT |
| K89 | AGTATTAACG |

K32 -> K14:K27:K39:K52:K53:K58:K74:K77:K89:K91:K92bis:K9:

| | |
|---|---|
| K32 | TTGCCTCCGT |
| K14 | AGGCGCGCAATG |
| K27 | AGGTGGCATG |
| K39 | ACGGGGTTCT |
| K52 | AAGGCGGAG |
| K53 | TACACGGTCT |
| K58 | AGGTGGAGGA |
| K74 | TCCGGAGTTT |
| K77 | ACGGCCAGA |
| K89 | AGTATTAACG |
| K91 | GGGCATGTTA |
| K92bis | ACGGCTG |
| K9 | TGGCAACAGG |

K34 -> K18:K25:K55:K76:K86:K89:

| | |
|---|---|
| K34 | ATAAGGACAT |
| K18 | TCCTGCGCA |
| K25 | GCCTGTCTAA |
| K55 | GTTTTGTAAG |
| K76 | GTCACCTGTTA |
| K86 | TTTGTCCTTC |
| K89 | AGTATTAACG |

K35 -> K13:K28:K42:K59:K5:K75:K88:K95:

| | |
|---|---|
| K35 | GTAAAGCG |
| K13 | TGAGTCGCA |
| K28 | TCTTCGCGTT |
| K42 | TTTATCGCTTTT |
| K59 | GATTCGGTGT |
| K5 | TCGCACCCTCA |
| K75 | TTTGGTTTCT |
| K88 | AGATTTGATAG |
| K95 | TCTCTCCTTTC |

K36 -> K13:K14:K16:K18:K2:K50:K5:K89:K98:K9:

| | |
|---|---|
| K36 | GCGTTCATTA |
| K13 | TGAGTCGCA |
| K14 | AGGCGCGCAATG |
| K16 | ACCCGCAATG |
| K18 | TCCTGCGCA |
| K2 | GCGCCAGTAG |
| K50 | GACTTCCGCA |
| K5 | TCGCACCCTCA |
| K89 | AGTATTAACG |
| K98 | TCCATGCGCA |
| K9 | TGGCAACAGG |

K37 -> K10:K11:K34:K44:K52:K73:K74:K9:

| | |
|---|---|
| K37 | TGTCGCCTGT |
| K10 | AAGACTCGACA |
| K11 | TGGACGCCTTC |
| K34 | TACAGGAATA |
| K44 | ACAGGGCGTCT |
| K52 | AAGGCGGAG |
| K73 | AGGTTAGCGA |
| K74 | TCCGGAGTTT |
| K9 | TGGCAACAGG |

K38 -> K17:K44:K4:K60bis:K64:K76:K82:

| | |
|---|---|
| K38 | GCAGTTCACT |
| K17 | ACGTCCCCT |
| K44 | ACAGGGCGTCT |
| K4 | TAGTGAGTTTT |
| K60bis | TGTCATCTCCc |
| K64 | GGGGTGAGAG |
| K76 | GTCACCTGTTA |
| K82 | GGAAGGTGAG |

K39 -> K16:K17:Kl:K32:K5:K61:K80:

| | |
|---|---|
| K39 | TCTTGGGGCA |
| K16 | ACCCGCAATG |
| K17 | ACGTCCCCT |
| K1 | AACCGCGTGTA |
| K32 | TGCCTCCGTT |
| K5 | TCGCACCCTCA |
| K61 | TGTCCCGCGTT |
| K80 | TAGTGCCGTA |

K4 -> K10:K38:K76:K85:

| | |
|---|---|
| K4 | TTTTGAGTGAT |
| K10 | AAGACTCGACA |
| K38 | TCACTTGACG |
| K76 | GTCACCTGTTA |
| K85 | ACACTG |

K40 -> K45:K56:K64:K70:K76:K77:K79:K94:

| | |
|---|---|
| K40 | ACAAGTCTCG |
| K45 | GAGCTGGTGTT |
| K56 | TGTGTTTGCT |
| K64 | GGGGTGAGAG |
| K70 | CTAGGAGCCTGG |
| K76 | GTCACCTGTTA |
| K77 | ACGGCCAGA |
| K79 | ATGTGTTTTG |
| K94 | GTCGTTCTCG |

K41 -> K18:K24:K25:K70:K76:

| | |
|---|---|
| K41 | TATGGACGGCT |
| K18 | TCCTGCGCA |
| K24 | TGCGCCGATC |
| K25 | GCCTGTCTAA |
| K70 | CTAGGAGCCTGG |
| K76 | GTCACCTGTTA |

K42 -> K35:K73:K88:K8:

| | |
|---|---|
| K42 | TTTTCGCTATTT |
| K35 | GCGAAATG |
| K73 | AGGTTAGCGA |
| K88 | AGATTTGATAG |
| K8 | AGGGGGGATAG |

K44 -> K10:K11:K18:K24:K25:K2:K38:K5:K76:

| | |
|---|---|
| K44 | TCTGCGGGACA |
| K10 | AAGACTCGACA |
| K11 | TGGACGCCTTC |
| K18 | TCCTGCGCA |
| K24 | TGCGCCGATC |
| K25 | GCCTGTCTAA |
| K2 | GCGCCAGTAG |
| K38 | TCACTTGACG |
| K5 | TCGCACCCTCA |
| K76 | GTCACCTGTTA |

K45 -> K2:K40:K53:K5:K6:K76:K77:K85:

| | |
|---|---|
| K45 | TTGTGGTCGAG |
| K2 | GCGCCAGTAG |
| K40 | GCTCTGAACA |
| K53 | TACACGGTCT |
| K5 | TCGCACCCTCA |
| K6 | AGCTTAATGT |
| K76 | GTCACCTGTTA |
| K77 | ACGGCCAGA |
| K85 | ACACTG |

K5 -> K13:K24:K35:K39:K45:K4:K64:K73:K80:K82:K8:K90:K99:

| | |
|---|---|
| K5 | ACTCCCACGCT |
| K13 | TGAGTCGCA |
| K24 | TGCGCCGATC |
| K35 | GCGAAATG |
| K39 | ACGGGGTTCT |
| K45 | GAGCTGGTGTT |
| K4 | TAGTGAGTTTT |
| K64 | GGGGTGAGAG |
| K73 | AGGTTAGCGA |
| K80 | TAGTGCCGTA |
| K82 | GGAAGGTGAG |
| K8 | AGGGGGGATAG |
| K90 | TGGGTGGTAAT |
| K99 | GATGGTGCTA |

K50 -> K13:K18:K24:K29:K36:K52:K74:K82:K98:

| | |
|---|---|
| K50 | ACGCCTTCAG |
| K13 | TGAGTCGCA |
| K18 | TCCTGCGCA |
| K24 | TGCGCCGATC |
| K29 | AGTCTGAATG |
| K36 | ATTACTTGCG |
| K52 | AAGGCGGAG |
| K74 | TCCGGAGTTT |
| K82 | GGAAGGTGAG |
| K98 | TCCATGCGCA |

K51 -> Kl:K55:K80:

| | |
|---|---|
| K51 | GCATTTTTGGT |
| K1 | AACCGCGTGTA |
| K55 | GTTTTGTAAG |
| K80 | TAGTGCCGTA |

K52 -> K11:K16:K1:K21:K24:K25:K2:K30:K32:K37:K50:K60bis:K61:K74:

| | |
|---|---|
| K52 | GAGGCGGAA |
| K11 | TGGACGCCTTC |
| K16 | ACCCGCAATG |
| K1 | AACCGCGTGTA |
| K21 | GCCTCCCCTTT |
| K24 | TGCGCCGATC |
| K25 | GCCTGTCTAA |
| K2 | GCGCCAGTAG |
| K30 | GTCCGGGTTA |
| K32 | TGCCTCCGTT |
| K37 | TGTCCGCTGT |
| K50 | GACTTCCGCA |
| K60bis | TGTCATCTCCc |
| K61 | TGTCCCGCGTT |
| K74 | TCCGGAGTTT |

K53 -> K10:K1:K45:K55:K56:K59:K79:K80:K96:

| | |
|---|---|
| K53 | TCTGGCACAT |
| K10 | AAGACTCGACA |
| K1 | AACCGCGTGTA |
| K45 | GAGCTGGTGTT |
| K55 | GTTTTGTAAG |
| K56 | TGTGTTTGCT |
| K59 | GATTCGGTGT |
| K79 | ATGTGTTTTG |
| K80 | TAGTGCCGTA |
| K96 | CGACGTGACA |

K55 -> K34:K51:K53:K6:K71:

| | |
|---|---|
| K55 | GAATGTTTTG |
| K34 | TACAGGAATA |
| K51 | TGGTTTTTACG |
| K53 | TACACGGTCT |
| K6 | AGCTTAATGT |
| K71 | GGCTTCTTAT |

K56 -> K16:K53:K85:K9:

| | |
|---|---|
| K56 | TCGTTTGTGT |
| K16 | ACCCGCAATG |
| K53 | TACACGGTCT |
| K85 | ACACTG |
| K9 | TGGCAACAGG |

K58 -> K18:K21:K32:K60bis:K76:K86:K98:

| | |
|---|---|
| K58 | AGGAGGTGGA |
| K18 | TCCTGCGCA |
| K21 | GCCTCCCCTTT |
| K32 | TGCCTCCGTT |
| K60bis | TGTCATCTCCc |
| K76 | GTCACCTGTTA |
| K86 | TTTGTCCTTC |
| K98 | TCCATGCGCA |

K59 -> K1:K24:K29:K35:K53:K5:K76:K85:

| | |
|---|---|
| K59 | TGTGGCTTAG |
| K1 | AACCGCGTGTA |
| K24 | TGCGCCGATC |
| K29 | AGTCTGAATG |
| K35 | GCGAAATG |
| K53 | TACACGGTCT |
| K5 | TCGCACCCTCA |
| K76 | GTCACCTGTTA |
| K85 | ACACTG |

K6 -> K36:K45:K6:K89:

| | |
|---|---|
| K6 | TGTAATTCGA |
| K36 | ATTACTTGCG |
| K45 | GAGCTGGTGTT |
| K89 | AGTATTAACG |

K60bis -> K20:K70:K74:K96:K99:

| | |
|---|---|
| K60bis | cCCTCTACTGT |
| K20 | TGGGATGGGTG |
| K70 | CTAGGAGCCTGG |
| K74 | TCCGGAGTTT |
| K96 | CGACGTGACA |
| K99 | GATGGTGCTA |

K61 -> K11:K14:K1:K20:K28:K30:K52:K61:K89:K8:

| | |
|---|---|
| K61 | TTGCGCCCTGT |
| K11 | TGGACGCCTTC |
| K14 | AGGCGCGCAATG |
| K1 | AACCGCGTGTA |
| K20 | TGGGATGGGTG |
| K28 | TCTTCGCGTT |
| K30 | GTCCGGGTTA |
| K52 | AAGGCGGAG |
| K89 | AGTATTAACG |
| K8 | AGGGGGGATAG |

K63 -> K16:K5:K76:K98:

| | |
|---|---|
| K63 | GTGGGTAGTA |
| K16 | ACCCGCAATG |
| K5 | TCGCACCCTCA |
| K76 | GTCACCTGTTA |
| K98 | TCCATGCGCA |

K64 -> K16:K38:K40:K5:K76:K94:K95:

| | |
|---|---|
| K64 | GAGAGTGGGG |
| K16 | ACCCGCAATG |
| K38 | TCACTTGACG |
| K40 | GCTCTGAACA |
| K5 | TCGCACCCTCA |
| K76 | GTCACCTGTTA |
| K94 | GTCGTTCTCG |
| K95 | TCTCTCCTTTC |

K70 -> K14:K18:K21:K2:K32:K34:K40:K41:K52:K71:K77:K92bis:K95:

| | |
|---|---|
| K70 | GGTCCGAGGATC |
| K14 | AGGCGCGCAATG |
| K18 | TCCTGCGCA |
| K21 | GCCTCCCCTTT |
| K2 | GCGCCAGTAG |
| K32 | TGCCTCCGTT |
| K34 | TACAGGAATA |
| K40 | GCTCTGAACA |
| K41 | TCGGCAGGTAT |
| K52 | AAGGCGGAG |
| K71 | GGCTTCTTAT |
| K77 | ACGGCCAGA |
| K92bis | ACGGCTG |
| K95 | TCTCTCCTTTC |

K71 -> K10:K55:K70:K82:

| | |
|---|---|
| K71 | TATTCTTCGG |
| K10 | AAGACTCGACA |
| K55 | GTTTTGTAAG |
| K70 | CTAGGAGCCTGG |
| K82 | GGAAGGTGAG |

K73 -> K13:K1:K28:K37:K42:K5:K76:K89:K99:

| | |
|---|---|
| K73 | AGCGATTGGA |
| K13 | TGAGTCGCA |
| K1 | AACCGCGTGTA |
| K28 | TCTTCGCGTT |
| K37 | TGTCCGCTGT |
| K42 | TTTATCGCTTTT |
| K5 | TCGCACCCTCA |
| K76 | GTCACCTGTTA |
| K89 | AGTATTAACG |
| K99 | GATGGTGCTA |

K74 -> K10:K21:K30:K32:K50:K52:K60bis:K74:

| | |
|---|---|
| K74 | TTTGAGGCCT |
| K10 | AAGACTCGACA |
| K21 | GCCTCCCCTTT |
| K30 | GTCCGGGTTA |
| K32 | TGCCTCCGTT |
| K50 | GACTTCCGCA |
| K52 | AAGGCGGAG |
| K60bis | TGTCATCTCCc |
| K76 -> | |
| K15:K20:K27:K34:K38:K3:K40:K41:K44:K45:K58:K59:K63:K64:K73:K82:K89:K90:K96:K99: | |
| K9: | |
| K76 | ATTGTCCACTG |
| K15 | CNGGTGGTTA |
| K20 | TGGGATGGGTG |
| K27 | AGGTGGCATG |
| K34 | TACAGGAATA |
| K38 | TCACTTGACG |
| K3 | TAGGTA |
| K40 | GCTCTGAACA |
| K41 | TCGGCAGGTAT |
| K44 | ACAGGGCGTCT |
| K45 | GAGCTGGTGTT |
| K58 | AGGTGGAGGA |
| K59 | GATTCGGTGT |
| K63 | ATGATGGGTG |
| K64 | GGGGTGAGAG |
| K73 | AGGTTAGCGA |
| K82 | GGAAGGTGAG |
| K89 | AGTATTAACG |
| K90 | TGGGTGGTAAT |
| K96 | CGACGTGACA |
| K99 | GATGGTGCTA |
| K9 | TGGCAACAGG |

K77 -> K24:K29:K40:K45:K70:K77:K80:K9:

| | |
|---|---|
| K77 | AGACCGGCA |
| K24 | TGCGCCGATC |
| K29 | AGTCTGAATG |
| K40 | GCTCTGAACA |
| K45 | GAGCTGGTGTT |
| K70 | CTAGGAGCCTGG |
| K80 | TAGTGCCGTA |
| K9 | TGGCAACAGG |

K78 -> K11:K24:K2:K80:K89:

| | |
|---|---|
| K78 | TTTGCGGCTA |
| K11 | TGGACGCCTTC |
| K24 | TGCGCCGATC |
| K2 | GCGCCAGTAG |
| K80 | TAGTGCCGTA |
| K89 | AGTATTAACG |

K79 -> K40:K53:K85:

| | |
|---|---|
| K79 | GTTTTGTGTA |
| K40 | GCTCTGAACA |
| K53 | TACACGGTCT |
| K85 | ACACTG |

K8 -> K17:K21:K42:K61:

| | |
|---|---|
| K8 | GATAGGGGGGA |
| K17 | ACGTCCCCT |
| K21 | GCCTCCCCTTT |
| K42 | TTTATCGCTTTT |
| K61 | TGTCCCGCGTT |

K80 -> K27:K38:K39:K41:K51:K53:K5:K77:K78:K85:K87:K91:K92bis:K9:

| | |
|---|---|
| K80 | ATGCCGTGAT |
| K27 | AGGTGGCATG |
| K38 | TCACTTGACG |
| K39 | ACGGGGTTCT |
| K41 | TCGGCAGGTAT |
| K51 | TGGTTTTTACG |
| K53 | TACACGGTCT |
| K5 | TCGCACCCTCA |
| K77 | ACGGCCAGA |
| K78 | ATCGGCGTTT |
| K85 | ACACTG |
| K87 | ATCGGCGTTT |
| K91 | GGGCATGTTA |
| K92bis | ACGGCTG |
| K9 | TGGCAACAGG |

K82 -> K11:K21:K38:K50:K5:K71:K76:K86:K95:

| | |
|---|---|
| K82 | GAGTGGAAGG |
| K11 | TGGACGCCTTC |
| K21 | GCCTCCCCTTT |
| K38 | TCACTTGACG |
| K50 | GACTTCCGCA |
| K5 | TCGCACCCTCA |
| K71 | GGCTTCTTAT |
| K76 | GTCACCTGTTA |
| K86 | TTTGTCCTTC |
| K95 | TCTCTCCTTTC |

K85-> K2:K45:K4:K56:K59:K79:K80:K97:

| | |
|---|---|
| K85 | GTCACA |
| K2 | GCGCCAGTAG |
| K45 | GAGCTGGTGTT |
| K4 | TAGTGAGTTTT |
| K56 | TGTGTTTGCT |
| K59 | GATTCGGTGT |
| K79 | ATGTGTTTTG |
| K80 | TAGTGCCGTA |
| K97 | TTGCAGTGGG |

K86 -> K11:K58:K82:

| | |
|---|---|
| K86 | CTTCCTGTTT |
| K11 | TGGACGCCTTC |
| K58 | AGGTGGAGGA |
| K82 | GGAAGGTGAG |

K87 -> K11:K24:K2:K80:K89:

| | |
|---|---|
| K87 | TTTGCGGCTA |
| K11 | TGGACGCCTTC |
| K24 | TGCGCCGATC |
| K2 | GCGCCAGTAG |
| K80 | TAGTGCCGTA |
| K89 | AGTATTAACG |

K89 -> K15:K28:K32:K36:K61:K6:K73:K76:K78:K87:K90:K91:K94:

| | |
|---|---|
| K89 | GCAATTATGA |
| K15 | CNGGTGGTTA |
| K28 | TCTTCGCGTT |
| K32 | TGCCTCCGTT |
| K36 | ATTACTTGCG |
| K61 | TGTCCCGCGTT |
| K6 | AGCTTAATGT |
| K73 | AGGTTAGCGA |
| K76 | GTCACCTGTTA |
| K78 | ATCGGCGTTT |
| K87 | ATCGGCGTTT |
| K90 | TGGGTGGTAAT |
| K91 | GGGCATGTTA |
| K94 | GTCGTTCTCG |

K9 -> K18:K25:K32:K56:K76:K79:K80:K91:K97:

| | |
|---|---|
| K9 | GGACAACGGT |
| K18 | TCCTGCGCA |
| K25 | GCCTGTCTAA |
| K32 | TGCCTCCGTT |
| K56 | TGTGTTTGCT |
| K76 | GTCACCTGTTA |
| K79 | ATGTGTTTTG |
| K80 | TAGTGCCGTA |
| K91 | GGGCATGTTA |
| K97 | TTGCAGTGGG |

K90 -> K16:K36:K5:K76:K89:

| | |
|---|---|
| K90 | TAATGGTGGGT |
| K16 | ACCCGCAATG |
| K36 | ATTACTTGCG |
| K5 | TCGCACCCTCA |
| K76 | GTCACCTGTTA |
| K89 | AGTATTAACG |

K91 -> K27:K32:K89:K91:K98:

| | |
|---|---|
| K91 | ATTGTACGGG |
| K27 | AGGTGGCATG |
| K32 | TGCCTCCGTT |
| K89 | AGTATTAACG |
| K98 | TCCATGCGCA |

K92bis -> K24:K32:K70:K80:

| | |
|---|---|
| K92bis | GTCGGCA |
| K24 | TGCGCCGATC |
| K32 | TGCCTCCGTT |
| K70 | CTAGGAGCCTGG |
| K80 | TAGTGCCGTA |

K94 -> K40:K64:K96:

| | |
|---|---|
| K94 | GCTCTTGCTG |
| K40 | GCTCTGAACA |
| K64 | GGGGTGAGAG |
| K96 | CGACGTGACA |

K95 -> K34:K35:K64:K70:K82:

| | |
|---|---|
| K95 | CTTTCCTCTCT |
| K34 | TACAGGAATA |
| K35 | GCGAAATG |
| K64 | GGGGTGAGAG |
| K70 | CTAGGAGCCTGG |
| K82 | GGAAGGTGAG |

K96 -> K13:K17:K25:K37:K38:K53:K60bis:K61:K76:K86:K94:

| | |
|---|---|
| K96 | ACAGTGCAGC |
| K13 | TGAGTCGCA |
| K17 | ACGTCCCCT |
| K25 | GCCTGTCTAA |
| K37 | TGTCCGCTGT |
| K38 | TCACTTGACG |
| K53 | TACACGGTCT |
| K60bis | TGTCATCTCCc |
| K61 | TGTCCCGCGTT |
| K76 | GTCACCTGTTA |
| K86 | TTTGTCCTTC |
| K94 | GTCGTTCTCG |

K97 -> K38:K85:K97:K9:

| | |
|---|---|
| K97 | GGGTGACGTT |
| K38 | TCACTTGACG |
| K85 | ACACTG |
| K9 | TGGCAACAGG |

K98 -> K11:K13:K14:K16:K18:K24:K27:K2:K36:K50:K5:K91:K98:

| | |
|---|---|
| K98 | ACGCGTACCT |
| K11 | TGGACGCCTTC |
| K13 | TGAGTCGCA |
| K14 | AGGCGCGCAATG |
| K16 | ACCCGCAATG |
| K18 | TCCTGCGCA |
| K24 | TGCGCCGATC |
| K27 | AGGTGGCATG |
| K2 | GCGCCAGTAG |
| K36 | ATTACTTGCG |
| K50 | GACTTCCGCA |
| K5 | TCGCACCCTCA |
| K91 | GGGCATGTTA |

K99 -> K5:K60bis:K73:K76:K98:

| | |
|---|---|
| K99 | ATCGTGGTAG |
| K5 | TCGCACCCTCA |
| K60bis | TGTCATCTCCc |
| K73 | AGGTTAGCGA |
| K76 | GTCACCTGTTA |
| K98 | TCCATGCGCA |

Loop sequences that show the best score for the complementarity are presented herein after:

Best scores for each sequence

| SEQ ID | SCORE RESULT | ALN | SEQ ID |
|---|---|---|---|
| K1 | 20.0 | ATGTGCGCCAA | K1 |
| | | \|\|\|.\|\|\|\| | |
| | | ACGGGGTTCT | K39 |
| K10 | 17.0 | ACAGCTCAGAA | K10 |
| | | :\|\|\|\|\| | |
| | | TGAGTCGCA | K13 |
| K10 | 17.0 | ACAGCTCAGAA | K10 |
| | | \|\|\|\|:\|\| | |
| | | TAGTGAGTTTT | K4 |
| K11 | 26.0 | CTTCCGCAGGT | K11 |
| | | \|:\|\|\|\|\|\|: | |
| | | ACAGGGCGTCT | K44 |
| K13 | 17.0 | ACGCTGAGT | K13 |
| | | \|\|\|\|\|: | |
| | | AAGACTCGACA | K10 |
| K14 | 24.0 | GTAACGCGCGGA | K14 |
| | | \|\|\|\|\|\| | |
| | | AGGCGCGCAATG | K14 |
| K15 | 18.0 | ATTGGTGGNC | K15 |
| | | ::\|\|\|\| \| | |
| | | GTCACCTGTTA | K76 |
| K16 | 18.0 | GTAACGCCCA | K16 |
| | | \|\|\|\|\| | |
| | | GTCCGGGTTA | K30 |
| K17 | 20.0 | TCCCCTGCA | K17 |
| | | :\|\|\|\|\|: | |
| | | AGGGGGGATAG | K8 |
| K18 | 20.0 | ACGCGTCCT | K18 |
| | | \|\|\|\|\|\| | |
| | | TCCTGCGCA | K18 |
| K18 | 20.0 | ACGCGTCCT | K18 |
| | | \|\|\|\|\|.\| | |
| | | TGCGCCGATC | K24 |
| K18 | 20.0 | ACGCGTCCT | K18 |
| | | \|\|\|\|\|\| | |
| | | TCCATGCGCA | K98 |
| K2 | 21.0 | GATGACCGCG | K2 |
| | | :\|\|\|\|.\|\|\| | |
| | | ATTACTTGCG | K36 |
| K20 | 19.0 | GTGGGTAGGGT | K20 |
| | | \|:\|\|\|\| | |
| | | ACGTCCCCT | K17 |
| K20 | 19.0 | GTGGGTAGGGT | K20 |
| | | \|:\|\|.\|\|\| | |
| | | TGCGCCGATC | K24 |
| K20 | 19.0 | GTGGGTAGGGT | K20 |
| | | \|\|\|\|::\|: | |
| | | GTCACCTGTTA | K76 |
| K21 | 23.0 | TTTCCCCTCCG | K21 |
| | | \|:\|.\|\|\|\|\| | |
| | | AGGTGGAGGA | K58 |
| K21 | 23.0 | TTTCCCCTCCG | K21 |
| | | \|\|\|\|\|:\| | |
| | | AGGGGGGATAG | K8 |
| K24 | 25.0 | CTAGCCGCGT | K24 |
| | | \|\|\|\|\|\|\|: | |
| | | ATCGGCGTTT | K78 |
| K24 | 25.0 | CTAGCCGCGT | K24 |
| | | \|\|\|\|\|\|\|: | |
| | | ATCGGCGTTT | K87 |
| K25 | 20.0 | AATCTGTCCG | K25 |
| | | \|.\|:\|\|\|\|: | |
| | | TCGGCAGGTAT | K41 |
| K27 | 21.0 | GTACGGTGGA | K27 |
| | | \|:\|\|\|\|\| | |
| | | GTCACCTGTTA | K76 |
| K28 | 21.0 | TTGCGCTTCT | K28 |
| | | \|\|.\|\|\|:\|\| | |
| | | AAGGCGGAG | K52 |
| K29 | 15.0 | GTAAGTCTGA | K29 |
| | | \|\|\|\|:\| | |
| | | GATTCGGTGT | K59 |
| K3 | 13.0 | ATGGAT | K3 |
| | | \|:\|\|\| | |
| | | TGCCTCCGTT | K32 |
| K3 | 13.0 | ATGGAT | K3 |
| | | \|\|\|\|: | |
| | | GTCACCTGTTA | K76 |
| K30 | 20.0 | ATTGGGCCTG | K30 |
| | | ::\|\|\|\|:: | |
| | | GTCCGGGTTA | K30 |
| K30 | 20.0 | ATTGGGCCTG | K30 |
| | | \|:\|\|:\|\| | |
| | | CTAGGAGCCTGG | K70 |
| K32 | 21.0 | TTGCCTCCGT | K32 |
| | | ::\|\|\|\|\| | |
| | | AGGTGGAGGA | K58 |
| K34 | 16.0 | ATAAGGACAT | K34 |
| | | \|\|\|\|\| | |
| | | TCCTGCGCA | K18 |
| K34 | 16.0 | ATAAGGACAT | K34 |
| | | \|\|\|\|\| | |
| | | GCCTGTCTAA | K25 |
| K35 | 14.0 | GTAAAGCG | K35 |
| | | \|\|\|\| | |
| | | TGAGTCGCA | K13 |
| K35 | 14.0 | GTAAAGCG | K35 |
| | | \|\|\|\| | |
| | | TCTTCGCGTT | K28 |
| K35 | 14.0 | GTAAAGCG | K35 |
| | | \|\|\|\| | |
| | | TTTATCGCTTTT | K42 |
| K35 | 14.0 | GTAAAGCG | K35 |
| | | \|\|\|\| | |
| | | TCGCACCCTCA | K5 |
| K36 | 21.0 | GCGTTCATTA | K36 |
| | | \|\|\|.\|\|\|\|: | |
| | | GCGCCAGTAG | K2 |
| K37 | 21.0 | TGTCGCCTGT | K37 |
| | | :\|\|\|:\|\|: | |
| | | TTGCAGTGGG | K97 |
| K38 | 19.0 | GCAGTTCACT | K38 |
| | | \|\|.\|\|:\|\| | |
| | | TCGGCAGGTAT | K41 |
| K39 | 20.0 | TCTTGGGGCA | K39 |
| | | \|\|\|\|.\|\|\| | |
| | | AACCGCGTGTA | K1 |
| K39 | 20.0 | TCTTGGGGCA | K39 |
| | | \|\|:\|.\|\|:\| | |
| | | CTAGGAGCCTGG | K70 |
| K4 | 18.0 | TTTTGAGTGAT | K4 |
| | | \|\|:\|\|\|\|: | |
| | | AAGACTCGACA | K10 |
| K40 | 19.0 | ACAAGTCTCG | K40 |
| | | \|\|\|:\|\| | |
| | | ACAGGGCGTCT | K44 |
| K41 | 20.0 | TATGGACGGCT | K41 |
| | | :\|\|\|\|:\| | |
| | | GCCTGTCTAA | K25 |
| K42 | 18.0 | TTTTCGCTATTT | K42 |
| | | \|\|:\|\|\|: | |
| | | AAGGCGGAG | K52 |
| K42 | 18.0 | TTTTCGCTATTT | K42 |
| | | \|:::\|.\|\|\|\|: | |
| | | AGGGGGGATAG | K8 |
| K44 | 26.0 | TCTGCGGGACA | K44 |
| | | :\|\|\|\|\|\|:\| | |
| | | TGGACGCCTTC | K11 |
| K45 | 21.0 | TTGTGGTCGAG | K45 |
| | | :\|:\|\|\|\|: | |
| | | GCGCCAGTAG | K2 |
| K5 | 23.0 | ACTCCCACGCT | K5 |
| | | :\|\|\|\|\|.\|\| | |
| | | GGGGTGAGAG | K64 |
| K50 | 20.0 | ACGCCTTCAG | K50 |
| | | \|:\|\|\|:\| | |
| | | AGGTGGAGGA | K58 |
| K51 | 17.0 | GCATTTTTGGT | K51 |
| | | \|\|::\|:\|\| | |
| | | CTAGGAGCCTGG | K70 |
| K52 | 21.0 | GAGGCGGAA | K52 |
| | | \|\|\|\|.\|:\|\| | |
| | | GCCTCCCCTTT | K21 |
| K52 | 21.0 | GAGGCGGAA | K52 |
| | | \|\|:\|\|\|.\|\| | |
| | | TCTTCGCGTT | K28 |
| K53 | 19.0 | TCTGGCACAT | K53 |
| | | \|\|\|\|.\|\|: | |
| | | AACCGCGTGTA | K1 |
| K55 | 15.0 | GAATGTTTTG | K55 |
| | | \|\|\|:::\| | |
| | | ACAGGGCGTCT | K44 |
| K56 | 17.0 | TCGTTTGTGT | K56 |
| | | :\|\|\|:::\|: | |
| | | TCGGCAGGTAT | K41 |
| K58 | 23.0 | AGGAGGTGGA | K58 |
| | | \|\|\|\|\|.\|:\| | |
| | | GCCTCCCCTTT | K21 |
| K59 | 20.0 | TGTGGCTTAG | K59 |
| | | :\|:\|\|\|\| | |
| | | TGCGCCGATC | K24 |
| K6 | 13.0 | TGTAATTCGA | K6 |
| | | :\|\|\|\| | |
| | | GAGCTGGTGTT | K45 |
| K6 | 13.0 | TGTAATTCGA | K6 |
| | | :\|.\|:\|\|:\| | |
| | | TAGTGAGTTTT | K4 |
| K6 | 13.0 | TGTAATTCGA | K6 |
| | | :\|\|.\|::\| | |
| | | TTGCAGTGGG | K97 |
| K60bis | 21.0 | cCCTCTACTGT | K60bis |
| | | \|\|.\|\|:\|\|\| | |
| | | GGAAGGTGAG | K82 |
| K60bis | 21.0 | cCCTCTACTGT | K60bis |
| | | \|\|\|:\|\|\| | |
| | | AGGGGGGATAG | K8 |
| K61 | 19.0 | TTGCGCCCTGT | K61 |
| | | \|.\|.\|\|\|\|:\| | |
| | | AGGGGGGATAG | K8 |
| K63 | 21.0 | GTGGGTAGTA | K63 |
| | | \|\|\|\|::\|:\| | |
| | | GTCACCTGTTA | K76 |
| K64 | 23.0 | GAGAGTGGGG | K64 |
| | | \|\|.\|\|\|\|\|: | |
| | | TCGCACCCTCA | K5 |
| K70 | 26.0 | GGTCCGAGGATC | K70 |
| | | \|:\|\|\|..\|\|\|:\| | |
| | | CTAGGAGCCTGG | K70 |
| K71 | 19.0 | TATTCTTCGG | K71 |
| | | \|:\|\|\|\| | |
| | | CTAGGAGCCTGG | K70 |
| K73 | 18.0 | AGCGATTGGA | K73 |
| | | \|\|\|.:\|:\| | |
| | | TGCGCCGATC | K24 |
| K74 | 20.0 | TTTGAGGCCT | K74 |
| | | \|\|\|\|\|\| | |
| | | TCCGGAGTTT | K74 |
| K75 | 18.0 | TCTTTGGTTT | K75 |
| | | \|\|:\|:\|\| | |
| | | CTAGGAGCCTGG | K70 |
| K76 | 21.0 | ATTGTCCACTG | K76 |
| | | \|\|\|\|\|:\| | |
| | | AGGTGGCATG | K27 |
| K76 | 21.0 | ATTGTCCACTG | K76 |
| | | \|:\|::\|\|\|\| | |
| | | ATGATGGGTG | K63 |
| K77 | 18.0 | AGACCGGCA | K77 |
| | | \|.\|\|.\|\|\| | |
| | | ACAGGGCGTCT | K44 |
| K77 | 18.0 | AGACCGGCA | K77 |
| | | \|\|\|\|\| | |
| | | TAGTGCCGTA | K80 |
| K78 | 24.0 | TTTGCGGCTA | K78 |
| | | \|\|\|\|\|\|\| | |
| | | TGCGCCGATC | K24 |
| K79 | 15.0 | GTTTTGTGTAK | 79 |
| | | \|\|:::\|:: | |
| | | ACAGGGCGTCT | K44 |
| K8 | 23.0 | GATAGGGGGGA | K8 |
| | | \|:\|\|\|\|\| | |
| | | GCCTCCCCTTT | K21 |
| K80 | 21.0 | ATGCCGTGAT | K80 |
| | | \|:\|\|::\|\|\| | |
| | | GATGGTGCTA | K99 |
| K82 | 25.0 | GAGTGGAAGG | K82 |
| | | \|\|\|.\|\|\|\|:\| | |
| | | TCTCTCCTTTC | K95 |
| K85 | 17.0 | GTCACA | K85 |
| | | \|:\|\|\|\| | |
| | | GATTCGGTGT | K59 |
| K86 | 20.0 | CTTCCTGTTT | K86 |
| | | \|::\|\|\|:\|: | |
| | | AGGGGGGATAG | K8 |
| K87 | 24.0 | TTTGCGGCTA | K87 |
| | | \|\|\|\|\|\|\| | |
| | | TGCGCCGATC | K24 |
| K88 | 15.0 | GATAGTTTAGA | K88 |
| | | \|\|:\|\|.\|\| | |
| | | GCCTGTCTAA | K25 |
| K89 | 15.0 | GCAATTATGA | K89 |
| | | \|.\|::\|:\|\| | |
| | | GATGGTGCTA | K99 |
| K9 | 18.0 | GGACAACGGT | K9 |
| | | \|\|\|\|\|\| | |
| | | GTCACCTGTTAK | 76 |
| K90 | 19.0 | TAATGGTGGGT | K90 |
| | | :\|\|.\|\|\| | |
| | | GCCTCCCCTTT | K21 |
| K91 | 19.0 | ATTGTACGGG | K91 |
| | | \|\|:\|.\|\|\| | |
| | | ACGTCCCCT | K17 |
| K92bis | 18.0 | GTCGGCA | K92bis |
| | | \|\|\|\|\| | |
| | | TAGTGCCGTA | K80 |
| K92bis | 18.0 | GTCGGCA | K92bis |
| | | \|:\|\|:\| | |
| | | ACGGCTG | K92bis |
| K94 | 17.0 | GCTCTTGCTG | K94 |
| | | \|:\|\|\|.\| | |
| | | TGGACGCCTTC | K11 |
| K95 | 25.0 | CTTTCCTCTCT | K95 |
| | | \|:\|\|\|\|.\|\|\| | |
| | | GGAAGGTGAG | K82 |
| K96 | 19.0 | ACAGTGCAGC | K96 |
| | | \|\|\|\|\|:.\|\| | |
| | | TGTCATCTCCc | K60bis |
| K97 | 21.0 | GGGTGACGTT | K97 |
| | | :\|\|:\|\|\|: | |
| | | TGTCCGCTGT | K37 |
| K98 | 20.0 | ACGCGTACCT | K98 |
| | | \|\|\|\|\|\| | |
| | | TCCTGCGCA | K18 |
| K98 | 20.0 | ACGCGTACCT | K98 |
| | | \|\|\|\|\|\| | |
| | | TCCATGCGCA | K98 |
| K99 | 22.0 | ATCGTGGTAG | K99 |
| | | \|.\|\|\|\|\|.\|\| | |
| | | TCGCACCCTCA | K5 |

We also checked, by EMSA at 10 nM and 200 nM, the ability to interact for RNA sequences of each family. 50 putative complexes have been screened. The affinity was lower than 10 nM for 7 complexes and for 3 palindromic sequences tested alone, 21 complexes showed an affinity between 10 and 200 nM.

The best results, where more than 50% of complexes were formed at a hairpin concentration of 10 nM is indicated on Table 1:

TABLE 1
Examples of complementarity of selected loop sequences. Names
are indicated on the left. Sequences are assembled in families
according to their homologies and complementarities. Each
family contains at least 3 sequences (5′ to 3′ from
left to right) able to match with one presumed complementary
sequence (5′ to 3′ from right to left) through five
contiguous base pairing. Putative complementary bases are
represented in grey. The first family shows the octa or
hexanucleotide autocomplementary sequences sharing a common
repeat purine/pyrimidine motif. Estimated KD (nM) are indicated
on the right.

| K14 | A G G C G C G C A A U G 5′ | KD |
|---|---|---|
| K14 | G U A A C G C G C G G A | |
| K18 | g A C G C G U C C U | |
| K98 | A C G C G U A C C U | |
| K24 | C U A G C C G C G U | |
| K2 | G A U G A C C G C G | |
| K11 | a c g U G G A C G C C U U C c g u 5′ | |
| K44 | u g c u c g U C U G C G G G A C A c g u g c a | 10-200 |
| K52 | u g c u G A G G C G G A A c g a g c a | 10-200 |
| K78 | u g c u c g U U U G C G G C U A c g a g c a | 10-200 |
| K14 | u g c c c g G U A A C G C G C G G A g a g c a | >200 |
| K76 | a c g a g c G U C A C C U G U U A g c u c g u 5′ | |
| K41 | u g c u u g U A U G G A C G G C U c g a g c a | <10 |
| K27 | u g c u c g G U A C G G U G G A c g a g c a | 10-200 |
| K58 | u g c u c g A G G A G G U G G A c g a g c a | <10 |
| K82 | u g c u c g G A G U G G A A G G c g a g c a | |
| K5 | a c g a g c U C G C A C C C U C A g c u c g u 5′ | |
| K64 | u g c u c g G A G A G U G G G G c g a g c a | |
| K90 | u g c u c g U A A U G G U G G G U c g a g c a | |
| K63 | u g c c c g G U G G G U A G U A c u g a g c | |
| K99 | u g c u c g A U C G U G G U A G c g a g c a | |
| K52 | a c g a g c A A G G C G G A G u c g u 5′ | |
| K32 | u g c u c g U U G C C U C C G U c g a g c a | <<10 |
| K37 | U G U C G C C U G U | |
| K50 | u g c u c g A C G C C U U C A G c g a g c a | <<10 |
| K36 | a c g a g c A U U A C U U G C G g c u c g u 5′ | |
| K13 | A C G C U G A G U | |
| K14 | G U A A C G C G C G G A | >200 |
| K16 | G U A A C G C C C A | |
| K18 | g A C G C G U C C U | >>200 |
| K50 | g A C G C C U U C A G | >>200 |
| K5 | A C U C C C A C G C U | ? |
| K98 | A C G C G U A C C U | >>200 |
| K32 | a c g a g c U G C C U C C G U U g c u c g u 5′ | |
| K52 | u g c u G A G G C G G A A c g a g c a | <<10 |
| K58 | u g c u c g A G G A G G U G G A c g a g c a | <<10 |
| K74 | U U U G A G G C C U | |
| K24 | a c g a g c U G C G C C G A U C g c c c g u 5′ | |
| K14 | G U A A C G C G C G G A | >200 |
| K18 | A C G C G U C C U | 10-200 |
| K98 | A C G C G U A C C U | 10-200 |
| K41 | U A U G G A C G G C U | 10-200 |
| K78 | U U U G C G G C U A | <<10 |

A short consensus motif composed of two intermolecular G-C base pairs in which the G were contiguous in the same loop and the C were on the complementary loop. A purine base (R) and preferentially a G was found at n−2 bases of the previous GG motif and consequently a pyrimidine (Y) was present at n+2 of the CC motif. The CC bases were separated from the pyrimidine by A, U or G. This CC(A/U/G)Y or CCDY/RHGG motif has been extended to the CCNY sequence even if the C was missing because it has not been tested and we have searched for these motifs RNGG and CCNY in the whole selected population of hairpins (110 sequences). We have observed that 52% of the sequences possessed either motif, which indicates that the population has evolved toward this degenerated sequence (Table 2)

TABLE 2
Best results of the screen by electrophoretic mobility shift assay of 50 putative
complexes and alignement of the RNA sequences which more than 50% of complex were formed
at a hairpin concentration of 10 nM. Estimation of the affinity is indicated on the right.
Complementary bases are underlined and common sequences are boxed.

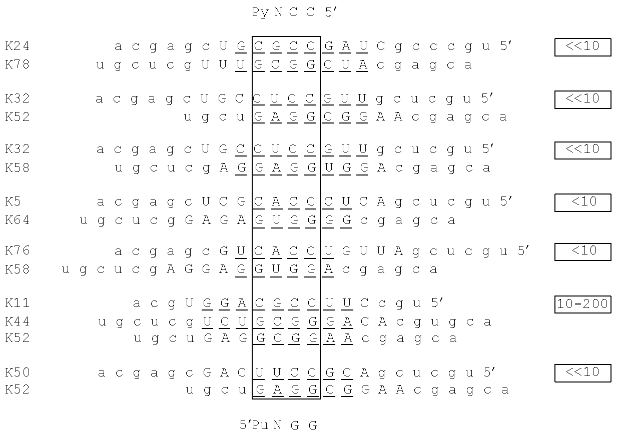

The K18, K14 and K98 loops contained autocomplementary octa or hexanucleotide sequences (Table 3). These autocomplementary sequences could be able to generate dimers as described for the DIS (dimerization initiation site of HIV-1, AMV). Interacting regions of these loops shared a common 4 GC contiguous base pair motif again. The putative interloop sequence was a linking of purine/pyrimidine. This purine/pyrimidine was repeated 3 times. The measure of the dissociation constants by EMSA for K14, K18 and K98 was uninterpretable because values where 50% of RNA was shifted into complexes, were less than 1 nM. In order to confirm the formation of complexes with high affinity and stability, we have performed thermal denaturation of RNA K18 complex (5′UGCUCGACGCGUCCUCGGCA). The melting temperature of K18 complex was studied at different concentrations and results indicated that dimers could formed. Tms at 1, 5 and 10 μM with 3 mM of magnesium were 60.6, 61.5 and 62.3° C., respectively. As the Tms observed were dependent on the K18 concentration, it means that Tms correspond to the fusion of K18-K18 complexes and not only to the fusion of the K18 hairpin. Values at 1, 5 or 10 μM were increased with 10 mM magnesium at 62.6, 65.4 and 66.5° C., respectively; showing that stability of the K18 duplex depends on magnesium.

TABLE 3
Sequence analysis of selection I for the CCNY and RNGG
motifs.

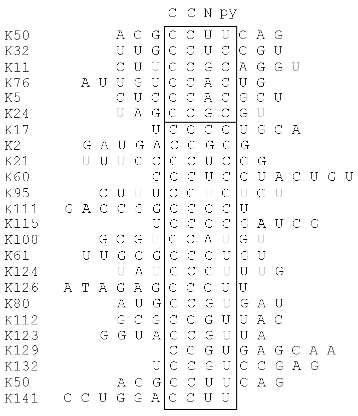

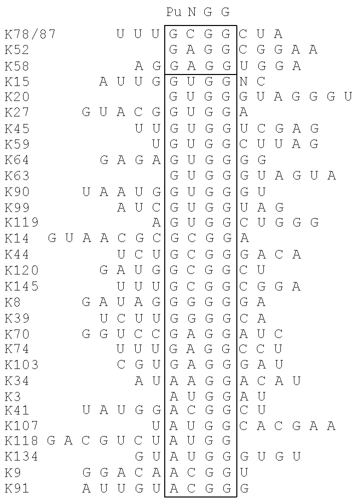

TABLE 4
Sequences and KD (10 nM) of the palindromic loop sequences

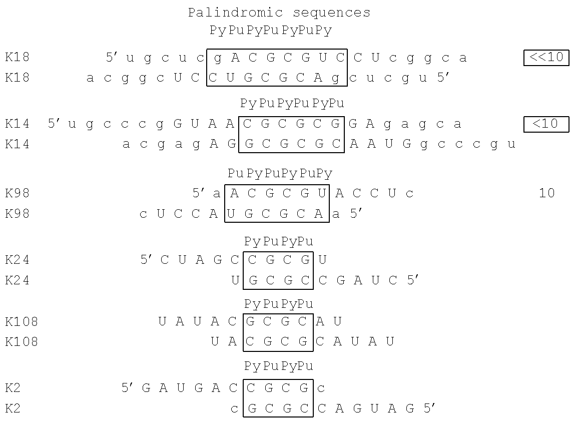

TABLE 5
The computational analysis of the 110
sequences of the selection confirms the
relevance of the YRYR and RYRY motifs.

| | RYRY sequences |
|---|---|
| | R Y R Y R Y R Y |
| K18 | A C G C G U C C U |
| K14 | G U A A C G C G C G G A |
| K98 | A C G C G U A C C U |
| K50 | A C G C C U U C A G |
| K16 | G U A A C G C C C A |
| K13 | A C G C U G A G U |
| K118 | G A C G U C U A U G G |
| K34 | A U A A G G A C A U |
| K80 | A U G C C G U G A U |
| K1 | A U G U G C G C C A A |
| K55 | G A A U G U U U U G |
| K108 | G C G U C C A U G U |
| K36 | G C G U U C A U U A |
| K112 | G C G C C G U U A C |
| K142 | A G A U C A G C G C G |
| K53 | U C U G G C A C A U |
| K107 | U A U G G C A C G A A |
| K27 | G C A C G G U G G A |
| K104 | A U U U A G C A U U |
| K123 | G G U A C C G U U A |
| K27 | G U A C G G U G G A |
| K134 | G U A U G G G U G U |
| K137 | U G G U A U U G U G |
| K139 | G U A U U G G G C A |
| K113 | G U G C U G A C A U |
| K96 | A C A G U G C A G C |
| | Y R Y R Y R Y R Y R Y |
| K102 | U A U A C G C G C A U |
| K144 | G G U U A U A C U |
| K41 | U A U G G A C G G C U |
| K89 | G C A A U U A U G A |
| K121 | U G C A C A U A U U |
| K17 | U C C C C U G C A |
| K117 | A U U U G U A C G U |
| K28 | U U G C G C U U C U |
| K61 | U U G C G C C C U G U |
| K78 | U U U G C G G C U A |
| K44 | U C U G C G G G A C A |
| K133 | C G U C C U G C G A |
| K145 | U U U G C G G C G G A |
| K6 | U G U A A U U C G A |
| K79 | U U U U G U G U A |
| K56 | U C G U U U G U G U |
| K59 | U G U G G C U U A G |
| K45 | U U G U G G U C G A G |
| K91 | A U U G U A C G G G |
| K131 | G G U U G U A G U U |
| K2 | G A U G A C C G C G |
| K24 | C U A G C C G C G U |
| K11 | C U U C C G C A G G U |
| K99 | A U C G U G G U A G |
| K103 | C G U G A G G G A U |
| K129 | C C G U G A G C A A |
| K110 | U C G U A G G C U U |
| K5 | A C U C C C A C G C U |
| K85 | G U C A C A |

Analysis of the frequencies of the four bases degenerated motives within 110 sequences gives the following results (the most represented are indicated from left to the right): YRYR, RYRY, YYRY, RYRR, YYYR, YRYY, RYYR, YRRY, YRRR, RYYY, RRYR, RRYY, RRRR, RRRY, YYYY, and YYRR.

In order to increase the number of usable kissing complexes and to determine other key determinants to the formation of RNA loop-loop complexes, as the preferential bases located at the stem-loop junction, a second in vitro selection has been performed around the previous described motif.

Selex II

A second SELEX has been performed with library A containing the consensus sequence CCNY against the biotinylated library B with the RNGG motif. Two rounds were done. We first analysed the candidates of library A. 45 sequences from the first round of selection and 43 from the second round were studied, respectively. All these hairpins contained the motif CCNY. Amplification of these CCNY candidates were performed with a new primer P20 containing a poly-T tail at the 5′ end. So, PCR products were in vitro transcribed into poly-A tailed RNA candidates. Poly-A candidates were immobilised on streptavidin beads by hybridization to a biotinylated complementary poly-T oligonucleotide. A new round of selection was performed against these candidates with the D library to identify NRNGGN partners of NCCNYN candidates. Primers of the D library have been changed compared to library A.

TABLE 6
Loop sequences of CCNY and RNGG that may interact with at least four central
nucleotidess after the Sekex II

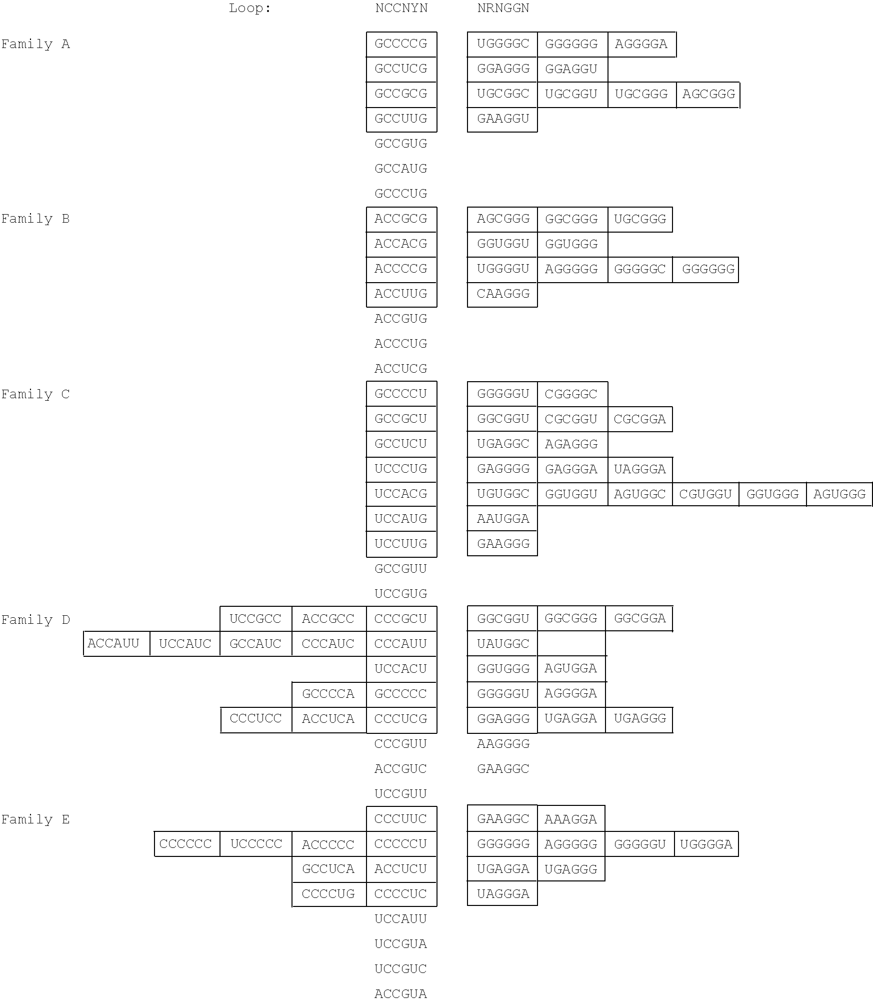

Results are depicted in Table 6. Analysis of N1 and N6 positions (N1CCNYN6) led to the emergence of three classes of sequences. Class A was composed of 24 sequences containing G-G bases at the stem loop junction. Class B was made up of 16 sequences presenting a consensus sequence A-G bases at these positions. Fifteen other sequences with a G-U or U-G bases at these positions composed the class C. Three families have emerged in regard to the N1-N6 composition of N1RNGGN6. Again, class A′ was enriched with G-G bases (21 sequences). Class B′ and class C′ were composed of 16 sequences containing U-C and 15 sequences containing G-U or U-G, respectively.

Sequences KC24: UGCUCGGCCCCGCGAGCA and KC23 UGCUCGGCCGUGCGAGCA were the most represented (8 and 7 times, respectively). Two other sequences UGCUCGACCGCGCGAGCA and UGCUCGACCCCCCGAGCA were found five and four times, respectively, in the NCCNYN hairpins, and for the NRNGGN partners, two sequences were found 10 times: KG51 GACGAGCUGGGGCGCUCGUC and KG114 GACGAGCGGGGGGGCUCGUC. The sequence KG71 GACGAGCUGGGGUGCUCGUC was represented 5 times.

Using thermal denaturation, the melting point of different complexes was defined at different concentration of magnesium showing that complexes were sensitive to the magnesium.

High affinity of these complexes has been shown by SPR experiments, for example: biotinylated KC24 (UGCUCGGCCCCGCGAGCA) was immobilized on streptavidin sensorships. KG51 (GACGAGCUGGGGCGCUCGUC) was injected at different concentrations that allowed to determine a Kd of 8 nM with 3 mM magnesium.

In conclusion, the in vitro selection used for selecting loop-loop complexes has allowed the identification of RNA kissing complexes sensitive to the magnesium and exhibiting high affinity.

### EXAMPLE 2

### Kissing Complex-Based Riboswitches for the Detection of Small Ligands

Aptamers are single chain nucleic acids obtained through a combinatorial process teemed SELEX {C. Tuerk, L. Gold, Science 1990, 249, 505-510; A. D. Ellington, J. W. Szostak, Nature 1990, 346, 818-822}. They display strong affinity and high specificity for a pre-determined target thanks to their 3D shape resulting from aptamer intramolecular folding that subsequently leads to optimized intermolecular interactions with the target molecule. Selection of RNA candidates to RNA hairpins led to hairpin aptamers whose loop is complementary to that of the target hairpin thus generating loop-loop interaction {a) F. Ducongé, J. J. Toulmé, R N A 1999, 5, 1605-1614; b) K. Kikuchi, T. Umehara, K. Fukuda, J. Hwang, A. Kuno, T. Hasegawa, S. Nishikawa, J. Biochem. (Tokyo) 2003, 133, 263-270; c) S. Da Rocha Gomes, E. Dausse, J. J. Toulmé, Biochem. Biophys. Res. Commun. 2004, 322, 820-826}. The stability of such so-called kissing complexes originates in Watson Crick base pairs of loop-loop helix but also in stacking interactions at the junctions between the loop-loop module and the double stranded stem of each hairpin partner {a) F. Beaurain, C. Di Primo, J. J. Toulmé, M. Laguerre, Nucleic Acids Res. 2003, 31, 4275-4284; b) I. Lebars, P. Legrand, A. Aime, N. Pinaud, S. Fribourg, C. Di Primo, Nucleic Acids Res. 2008, 36, 7146-7156; c) H. Van Melckebeke, M. Devany, C. Di Primo, F. Beaurain, J. J. Toulmé, D. L. Bryce, J. Boisbouvier, Proc. Natl. Acad. Sci. USA 2008, 105, 9210-9215}. Indeed the binding of the Trans-Activating Responsive (TAR) RNA imperfect stem loop element of the Human Immunodeficiency Virus to a hairpin aptamer generating a 6 base pair loop-loop helix was characterized by a melting temperature 20° C. higher than that of the complex between TAR and an antisense oligomer giving rise to the same 6 base pair duplex {F. Ducongé, C. Di Primo, J. J. Toulmé, J. Biol. Chem. 2000, 275, 21287-21294}. We exploited the potential of RNA hairpins to discriminate between folded and linear structures for designing aptamer-based sensors.

Riboswitches are RNA modules identified in prokaryotes that are constituted of a sensor including the binding site for a small ligand, that responds to the association with the ligand by a conformational change {a) A. Serganov, E. Nudler, Cell 2013, 152, 17-24; b) B. J. Tucker, R. R. Breaker, Curr. Opin. Struct. Biol. 2005, 15, 342-348}. The sensor is the functional equivalent of an aptamer and displays similar properties as regards specificity, in particular. One might derive an aptamer into a molecule switching between a folded and an open conformation in the presence and in the absence of its cognate target, respectively. Several examples of such aptamers have been described in the literature. However, no study describes an aptamer which can switch to a conformation comprising a loop able to form a kissing complex once said aptamer is bound to its target molecule.

We exploited the formation of kissing complexes for sensing the presence of a ligand that is specifically recognized by a hairpin aptamer. The aptamer is engineered in such a way that the binding of the small molecule shifts its conformation from an unfolded to a folded (hairpin) shape, hence its name aptaswitch. The recognition of the folded structure is ensured by a second hairpin able to form a kissing complex with the aptaswitch. This second molecule is termed aptakiss. Therefore the formation of the aptaswitch-aptakiss complex signals the presence of the small molecule.

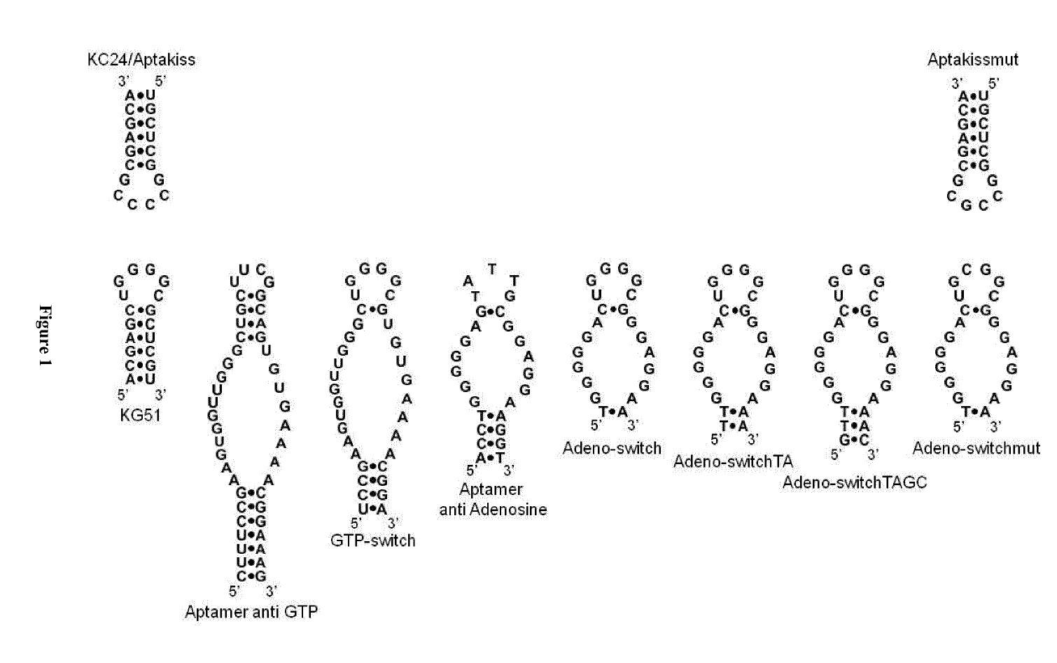

We validated this concept with aptamers previously raised against nucleic acid base derivatives, namely GTP and adenosine. Both of them exhibit a purine rich central loop that constitutes the binding site of the ligand (FIG. 1). We demonstrated that our strategy can be adapted to either RNA or DNA aptamers. As described in the following, aptaswitch-aptakiss combination allowed the specific and quantitative detection of the target ligand by either surface plasmon resonance (SPR) or by fluorescence anisotropy using an immobilized or a fluorescently labelled aptakiss, respectively.

We tailored our sensors on the basis of KC24-KG51, a RNA-RNA kissing complex previously identified in EXAMPLE 1 and characterized by a low Kd (5 nM at room temperature, in 20 mM HEPES buffer pH 7.4, containing 140 mM K⁺, 20 mM Na⁺ and 10 mM Mg⁺⁺) as evaluated by SPR. These hairpins potentially form a 6 bp loop-loop helix, including 5 GC and 1 GU pairs. KC24 was truncated down to 18 nt thus generating the aptakiss used in this study with a 6 bp stem and a 6 nt loop (FIG. 1). Aptaswitches were engineered by inserting the KG51 loop sequence 5′CUGGGGCG prone to interaction with the KC24 (aptakiss) loop, in the apical loop of previously described imperfect hairpin aptamers raised against either GTP or adenosine, thus generating GTPswitch and adenoswitch, respectively (FIG. 1 and Table 7).

TABLE 7
sequences of oligonucleotides used in the aptaswitch engineering experiments

| | |
|---|---|
| Aptakiss-biot | 5′ U G C U C G G C C C C G C G A G C A - biot |
| Aptakiss-TR | 5′ U G C U C G G C C C C G C G A G C A - TR |
| Aptakissmut-biot | 5′ U G C U C G G C C G C G C G A G C A - biot |
| Aptakissmut-TR | 5′ U G C U C G G C C G C G C G A G C A - TR |
| KG51 | 5′ A C G A G C U G G G G C G C U C G U |
| GTPswitch | 5′ U C C G A A G U G G U U G G G C U G G G G C G U G U G A A A A C G G A |
| Adenoswitch | 5′ T G G G G G A C U G G G G C G G G A G G A A |
| Adenoswitchmut1 | 5′ T G G G G G A C U G C G G C G G G A G G A A |
| AdenoswitchTA | 5′ T T G G G G G A C U G G G G C G G G A G G A A A |
| AdenoswitchTAGC | 5′ G T T G G G G G A C U G G G G C G G G A G G A A A C |
| AdenoswitchTAGCmut2 | 5′ G T T G A G G A A C U G G G G C G G G A G G A A A C |
| Aptamer anti-GTP | 5′ C U U U C C G A A G U G G U U G G G C U G C U U C G G C A G U G U |
| | G A A A A C G G A A A G |
| Aptamer anti-Adenosine | 5′ A C C T G G G G G A G T A T T G C G G A G G A A G G T |

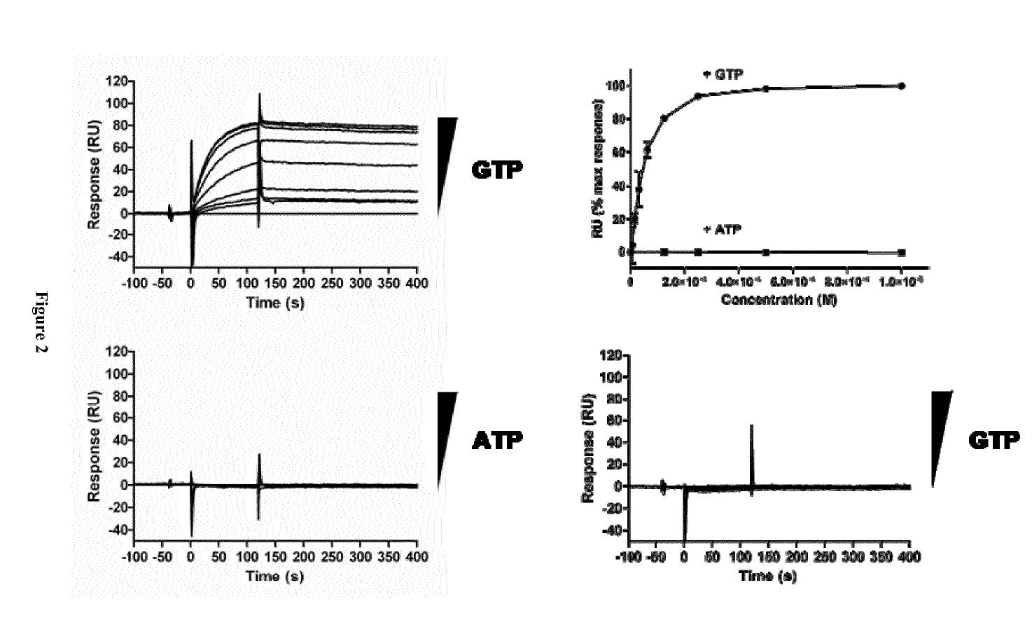

The previously described anti-GTP RNA aptamer was converted into a GTPswitch by substituting the 5′CUGGGGCG sequence to the original apical part of the aptamer that was demonstrated not to interact with GTP. In addition the aptamer stem was reduced to 4 base pairs, the central 27 nt of the resulting oligonucleotide likely remaining as a large non structured single-stranded internal loop. This GTPswitch did not give rise to a detectable SPR signal when flown over a chip on which the aptakiss was immobilized. In contrast injection of a preincubated GTPswitch/GTP mixture led to a signal the amplitude of which increased with GTP concentration up to 0.25 mM (FIG. 2): at 8 microM a signal of 10 RU was detected under our experimental conditions. This is likely due to the recognition by the aptakiss of the structure induced by GTP binding to the GTPswitch. No SPR signal is observed when ATP that is not recognized by the aptamer is substituted for GTP (FIG. 2). It is not detected either when a point-mutated apatkiss that introduces a G-G missmatch in the loop-loop helix (FIG. 1) is immobilized on the sensor chip (FIG. 2). These experiments demonstrate the high specificity of the sensor and underline the role played by ligand-aptaswitch interactions on the one hand, by kissing complex formation on the other hand, thus validating our design.

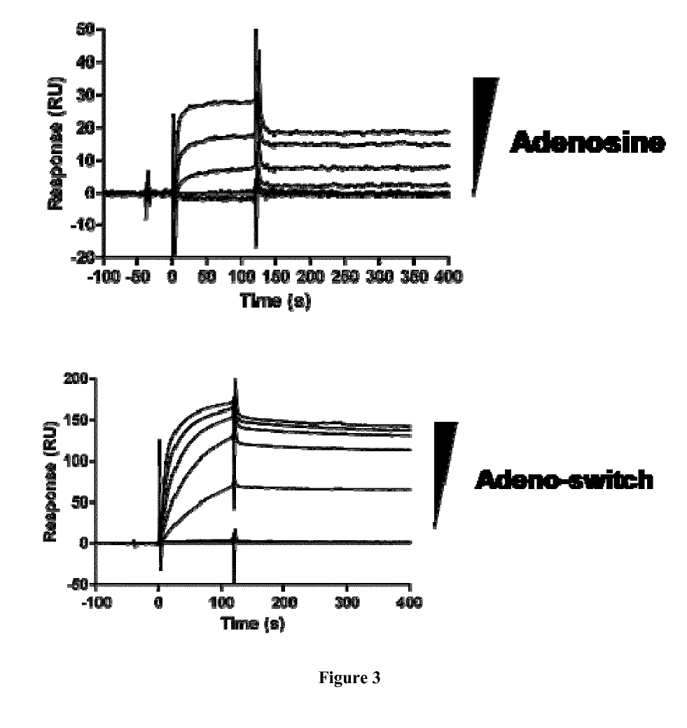

In the second case the same approach was used except that it resulted in a chimeric aptaswitch as the starting point was a DNA aptamer to adenosine. The 5′CUGGGGCG sequence was substituted to the original apical part of the parent hairpin (FIG. 1). The stem of the parent aptamer was then drastically shortened leaving only a single potential AT pair at the very bottom of the structure and leading to a 21 nt long adenoswitch. Its properties were then investigated by SPR against the biotinylated aptakiss immobilized on the chip. As above for the GTP switch the amplitude of the signal was correlated to the concentration of added adenosine, no resonance being detected when the adenoswitch was injected alone (FIG. 3).

At a fixed adenosine concentration the SPR signal also increased with the adenoswitch concentration, indicating that the adenosine adenoswitch complex was the species recognized by the immobilized aptakiss and not the free adenoswitch. The sensor constituted by the aptakiss-adenoswitch tandem proved to be specific: no signal was detected when adenosine was substituted by inosine that is not recognized by the parent aptamer. The introduction of a point mutation either in the aptakiss or in the adenoswitch loop also resulted in no signal demonstrating the absolute requirement of kissing interaction for a functional sensor. Interestingly no detection was observed by the aptakissmut-adenoswitchmut combination even though complementarity of the two loops is ensured. Indeed the stability of kissing complexes is highly dependent on loop sequences and this particular loop-loop duplex is significantly less stable than the parent one.

Previous NMR study demonstrated stacking of the adenosine ring with purine pairs of the central loop of the aptamer. This contributes to shaping the apical part of the adenoswitch as a loop prone to kissing recognition. The sensitivity of the sensor will depend on the equilibrium between the unfolded and folded state of the aptaswitch i.e. to the binding constant of the aptaswitch for its ligand. In other words the extent of the stacking contribution brought by the adenosine to formation of the hairpin structure of the adenoswitch recognized by the aptakiss will impact the detection. In the extreme case the hairpin is formed in the absence of the ligand, the aptakiss will bind the free adenoswitch.

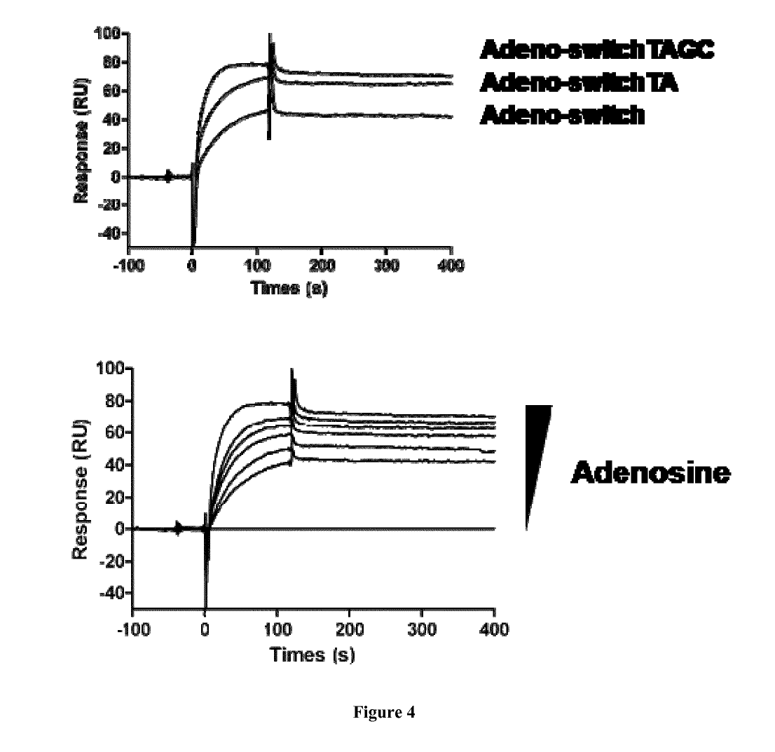

One might therefore expect that the sensor response will be related to the stability of the adenoswitch hairpin in the absence of the ligand as previously reported for an aptamer-based molecular beacon. In order to test this hypothesis we evaluated the properties of adenoswitch variants in which we added one or two base pairs at the bottom of the stem (FIG. 1). As shown in FIG. 4 the SPR response for a fixed adenosine concentration increased in the order adenoswitch <adenoswitchTA<adenoswitchTAGC, i.e. with the number of base pairs in the hairpin stem. Indeed the latter variant allowed the detection of adenosine at a concentration of 0.125 mM (FIG. 4) compared to 2 mM for the parent adenoswitch (FIG. 3). The increased sensitivity was not achieved at the expense of the specificity: the adenoswitchATGC (otherwise referred to as ADOsw1′ in the following) did not recognise inosine and no signal was observed on a chip functionalised with aptakissmut.

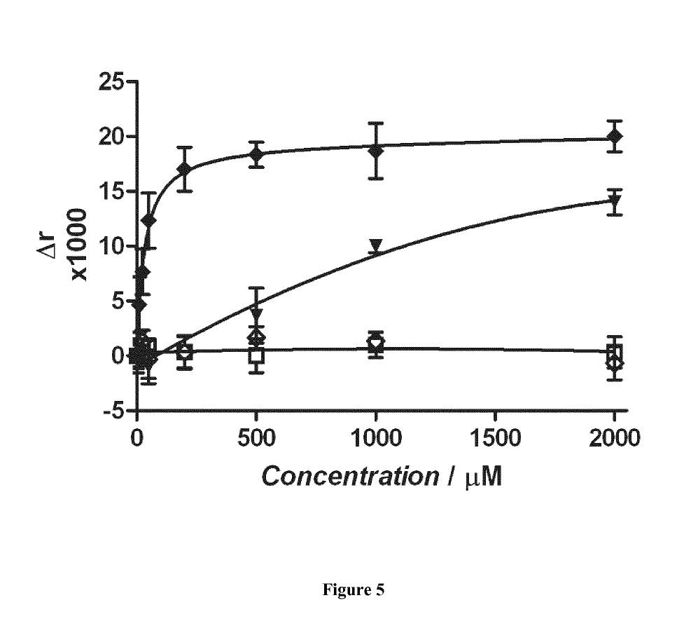

We further considered the possibility to detect aptakiss-aptaswitch complexes in solution by using fluorescence anisotropy (FA). The chimeric adenoswitch described above was employed to this end using a Texas red (TR) 3′ end conjugated aptakiss as fluorescent probe (aptakiss-TR). The binding of the aptakiss to the adenoswitch will result in increased overall size and consequently in increased FA. In the presence of 10 nM adenoswitch, the FA signal (r) of aptakiss-TR (10 nM) was enhanced when adenosine was added to the reaction mixture. The FA change, i.e. Δr=r−r₀ where r₀ is the anisotropy in absence of ligand, reached ˜0.015 at the 2 mM adenosine concentration (FIG. 5).

Dose response curves were then established with the optimized adenoswitchTAGC. As shown in FIG. 5, the sensitivity was greatly improved relatively to the parent adenoswitch. An apparent dissociation constant of 35 μM was obtained for the ternary complex formed with adenoswitchTAGC, close to the 5-10 μM value reported for the original aptamer. The FA response was invariant upon adenosine addition when 2 As on the 5′ side of the internal purine loop that are part of the adenosine binding site were exchanged for 2 Gs (adenoswitchTAGCmut2). As expected no FA variation was observed either upon addition of inosine that does not bind to the adenosine aptamer. This confirms that the signal transduction is dependent on the adenosine binding to the aptamer domain of the adenoswitch.

In conclusion we engineered aptamers against purine derivatives for generating switching sensors. The structure of the aptamers was optimized in such a way that the hairpin shape is adopted exclusively in the presence of the cognate ligand i.e. GTP or adenosine. In addition we successfully substituted part of the apical loop of the parent aptamers by a short RNA sequence prone to loop-loop interaction with a hairpin aptakiss without altering significantly the binding properties of the aptaswitch. The formation of the aptaswitch-aptakiss complex signaling the presence of the ligand can be monitored by various techniques (fluorescence, SPR) that could be automated. The same aptakiss can be used for detecting any aptaswitch the loop of which is appropriately modified with the complementary sequence as demonstrated here for GTP and adenosine.

Other aptamer-based sensors were described for the detection of adenosine. For the aptaswitch-based fluorescence anisotropy assay, the limit of detection for adenosine was estimated to be about 10 μM, in the same range as those commonly reported with fluorescent aptasensing methods (excluding sophisticated amplification-based biosensors).

### EXAMPLE 3

### Characterization of Kx1, Kx2, Kx3 and Kx4 Complexes

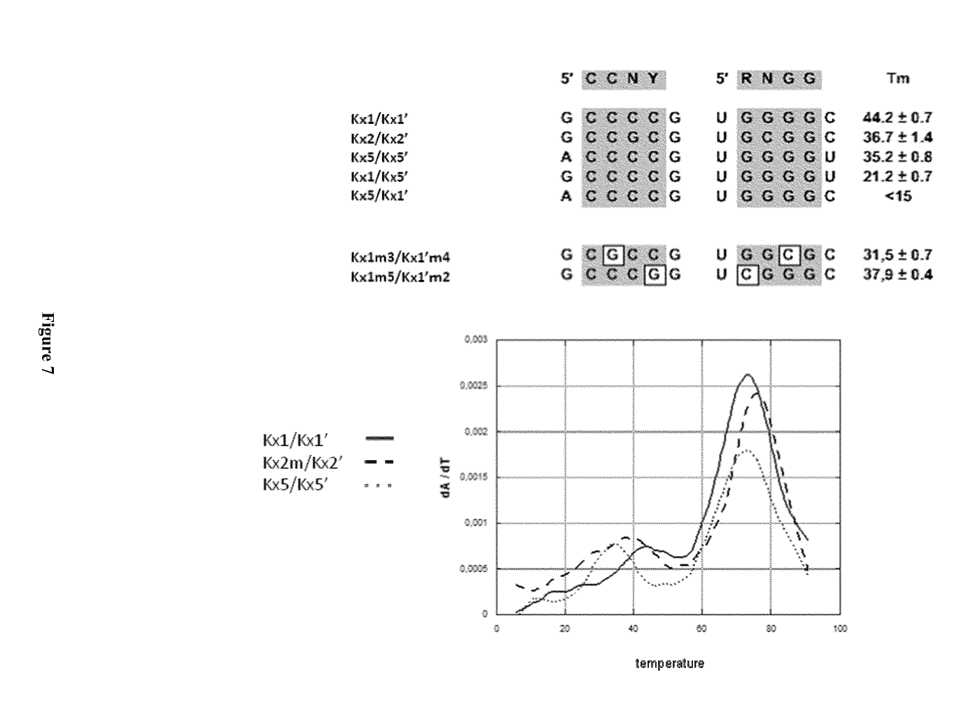

We then undertook the characterization of a few kissing couples that will be secondarily used for designing aptaswitches that could allow the simultaneous detection of several ligands. To this end we chose four sequences that for sake of simplicity we termed Kx1 to Kx4 and their kissing complement Kx1′ to Kx4′. Kx1, Kx1′, Kx2 and Kx2′, correspond to the above KC24, KG51, KC28 and KG49 sequences, respectively. Kx3, Kx4, Kx3′ and Kx4′ have the following sequences, respectively: GGUCGGUCCCAGACGACC (loop sequence GUCCCAGA), GGUUUCAGGGCAGUGAUGUUGCCCCUCGGAAGAUAACC (loop sequence GUGAUGU), CGAGCCUGGGAGCUCG (loop sequence CUGGGA) and CCUGACAUCACCAGG (loop sequence ACAUCAC). Hairpins Kx1 to Kx4 were chemically synthesized with a 3′ biotinyl residue thus allowing their immobilization on a streptavidin sensor chip. The kissing motif is displayed in the context of a hairpin with a 6 nt stem and a 6 nt loop. In addition, in order to prevent the formation of extended duplexes that might have occurred in the original pool due to the presence of an identical stem for every candidate hairpin, we designed partners with different stems. We first investigated in details one of these complexes: Kx1-Kx1′ displaying the loop sequences 5′GCCCCG and 5′UGGGGC, respectively (the stem used in this experiment for Kx1′ being formed with two complementary sequences: 5′-ACGAGC . . . GCUCGU-3′; the stem for Kx1 is formed with two complementary sequences: 5′-UGCUCG . . . CGAGCA-3′). We checked the effect of point mutations on complex stability by UV absorbance-monitored thermal denaturation and by surface plasmon resonance (SPR). In a buffer containing 3 mM Mg²⁺ the Tm of the transition for the Kx1-Kx1′ parent complex was 44.2+0.7° C. (FIG. 7). SPR analysis with immobilized Kx1 led to a Kd of 6.9+1.1 nM (FIG. 7). Substituting the 5′ U in Kx1′ loop by C in order to generate an additional GC pair in the loop-loop duplex actually resulted in an increase of the Kd (18+1.1 nM). Other point modifications in Kx1 combined with complementary mutations in Kx1′ led to an even more drastic effect: for instance inversion of the second GC pair of the loop-loop duplex resulted in a weak complex (Kx1m3/Kx1′m4 complex: Tm=31.5±0.7° C., compared to the wild type, pointing out both the significance of the CCNY/RNGG motif and the interest of our selection approach. In addition, different complexes with sequences Kx5 (ACCCCG) and Kx5′ (UGGGGU) were also evaluated (FIG. 7) and formed stable complexes.

We investigated by SPR the compatibility of the four pre-selected kissing complexes by immobilizing the biotinylated hairpins Kx1 to Kx4 on different channels of the biochip. Individual solutions of Kx1′, Kx2′, Kx3′ or Kx4′ were flown over the chip. A nice resonance signal was obtained as expected for each cognate combination Kx1-Kx1′, Kx2-Kx2′ and Kx4-Kx4′ (FIG. 7). In contrast no signal was observed for any other combination, except Kx3′-Kx1 (not shown); consequently we no longer used the Kx3-Kx3′ complex and restricted ourselves to the 3 other combinations for the design of kissing complex-based aptasensors.

### EXAMPLE 4

### Kissing Complex-Based Riboswitches for the Detection of Theophylline

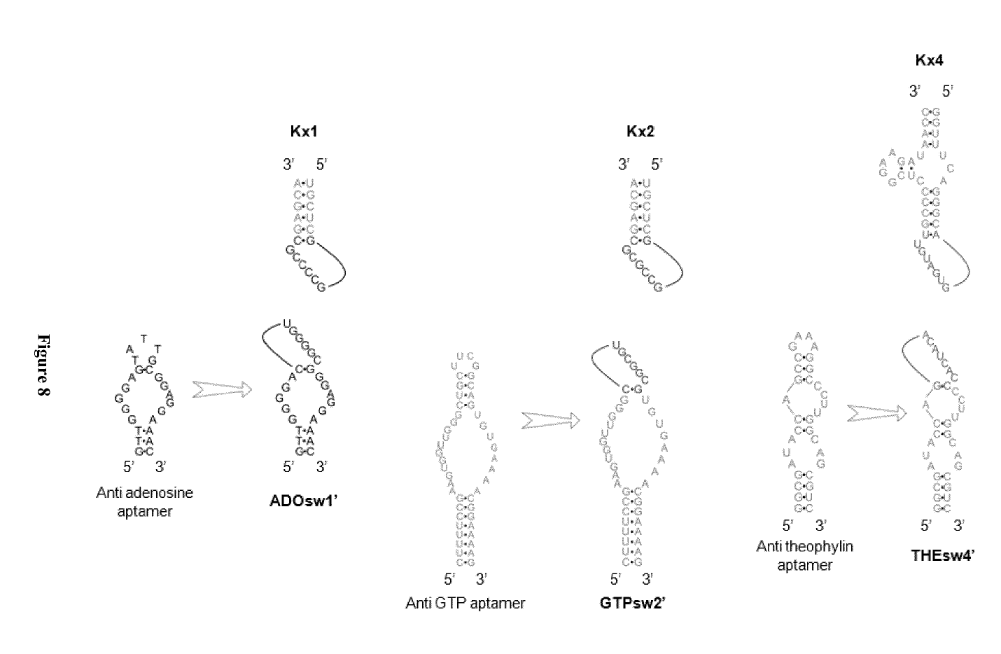

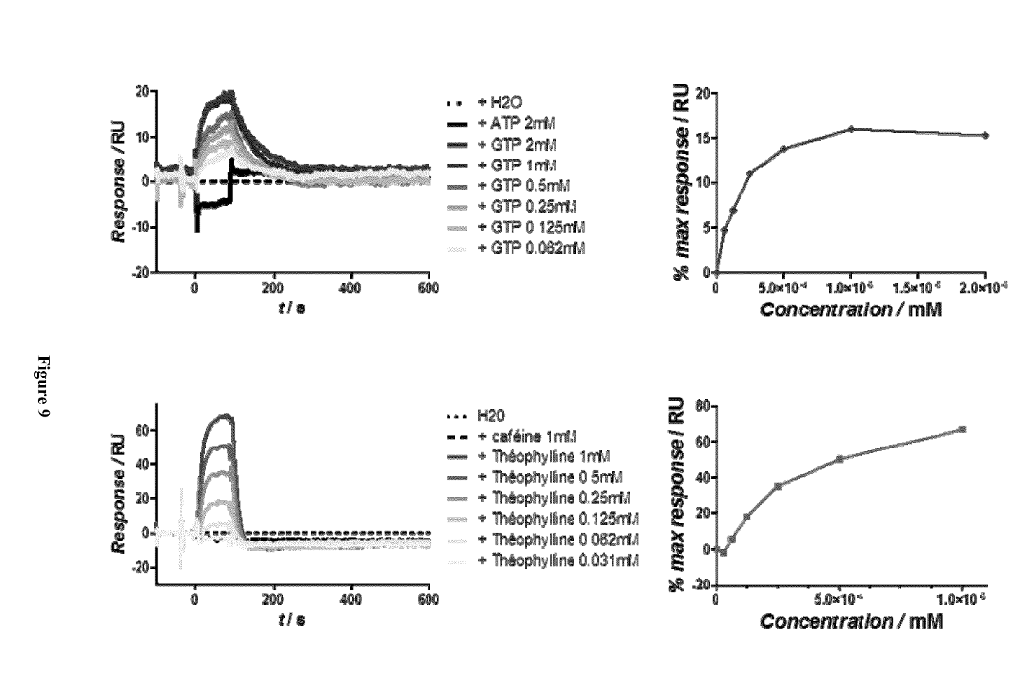

As described above aptamers organized as imperfect hairpins whose apical loop is not involved in the binding of their cognate ligands can potentially be engineered into aptaswitches. To this end we need first to substitute a short sequence prone to kissing interaction to the original aptamer loop. We introduced the loops of Kx1′, Kx2′ or Kx4′ in aptamers previously selected against adenosine, GTP or theophylline, thus generating ADOsw1 GTPsw2′ and THEsw4′, respectively (FIG. 8). These aptamers are characterized by a purine rich internal loop that constitutes the binding site of their respective ligand. We taylored the short double-stranded regions above and below the central loop in such a way that the aptaswitch conditionally fold into a hairpin upon addition of the cognate aptamer target. The aptaswitch-ligand complex is then recognized by the aptakiss Kx1, Kx2 or Kx3 whereas the unfolded aptamer is not. ADOsw1′ was previously characterized above (Adeno-switchTAGC): a specific SPR or fluorescence signal was observed upon the simultaneous addition of adenosine and Kx1 whereas no signal was detected in the presence of inosine. GTPsw2′ is derived from an aptaswitch described above, by substituting the Kx2′ loop to the formerly used Kx1′. Its specific responsiveness relative to GTP and to the aptakiss are retained: a dose-dependent SPR signal was observed upon addition of GTP whereas no resonance was seen with ATP (FIG. 9). Designing THEsw4′ required trial and errors: the best aptaswitch (FIG. 8) contains x and y putative base pairs below and above the internal loop. A mixture of THEsw4′ and of theophylline induces a SPR signal when flown over a chip on which Kx4 was immobilized (FIG. 9). No signal was detected in the presence of caffeine indicating that the specificity of binding is retained. However the affinity of THEsw4′ for theophylline is reduced compared to the parent aptamer. It should be pointed out that the three aptaswitch-aptakiss complexes were evaluated under the same ionic conditions, a prerequisite for multiplex analysis, even though this does not correspond to the best medium for all complexes.

### EXAMPLE 5

### Simultaneous Detection of Ligands by SPR and by Fluorescence Anisotropy

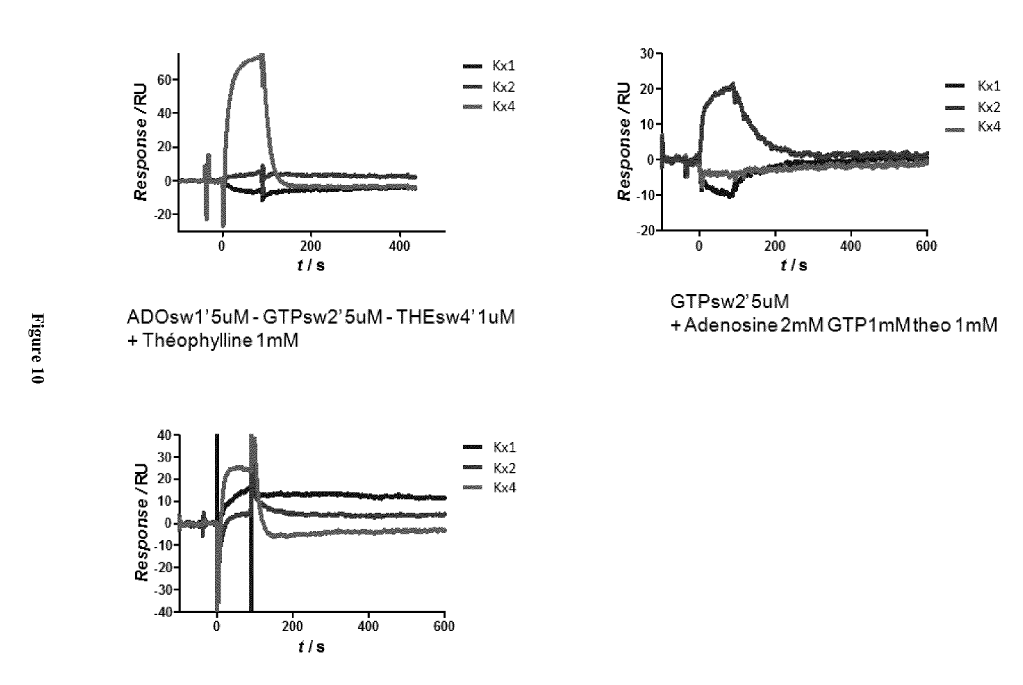

To this end we used a SPR streptavidin biochip with four channels. Biotinylated Kx1, Kx2 and Kx4 were immobilized on channels 1, 2 and 3, respectively, the 4th one was saturated with biotinylated linker and used as a control. As shown (FIG. 10) flowing ADOsw1 GTPsw2′ or THEsw4′ with saturating conditions of the cognate ligand, adenosine, GTP or theophylline, respectively, under saturating conditions resulted exclusively in a signal on the corresponding functionalized channel i.e. 1, 2 or 4, respectively. More complex mixtures were then tested: the simultaneous presence of the three aptaswitches allowed the specific detection of one ligand: for instance 1 mM theophylline added to 5 μM ADOsw1′+5 μM GTPsw2′+1 μM THEsw4′ yielded a signal exclusively on channel 3 (FIG. 10). Similar results were obtained for 2 mM GTP (not shown). The mixture with one aptaswitch and three ligands also generated a specific response: for instance 5 μM GTPsw2′ with 2 mM adenosine+1 mM GTP+1 mM theophylline gave a signal only on channel 2 (FIG. 10). The use of either ADOsw1′ or THEsw4′ with the three ligands also allowed the detection of the cognate ligand (not shown). Finally we injected over the 4 channel chip a mixture of three aptaswitches and three ligands and observed a signal on the three channels bearing the three different aptakisses (FIG. 10).

### EXAMPLE 6

### Use of a DNA Aptaswitch—RNA Aptakiss Couple

In the above, we describe chimeric DNA-RNA, which means that the small molecule (adenosine) bind to the region of the aptaswitch derived from the DNA aptamer previously identified against adenosine. RNA loop able to form a kissing complex with the aptakiss RNA has been added to this DNA aptamer. This constraint is related to the fact that kissing complexes involving two DNA loops have not yet been described. In contrast, a kissing complex involving RNA and DNA loop interaction has been selected (Darfeuille, F., Sekkai, D., Dausse, E., Kolb, G., Yurchenko, L., Boiziau, C., and Toulme, J. J. (2002) Comb Chem High Throughput Screen 5, 313-25). This SELEX from a random DNA library was directed against the TAR RNA hairpin structure of HIV-1. Selected candidates were tested for their affinity to TAR by EMSA (electrophoresis mobility shift assay) and one of them, called DII21, showed a 20 nM affinity at room temperature in the presence of 3 mM magnesium.

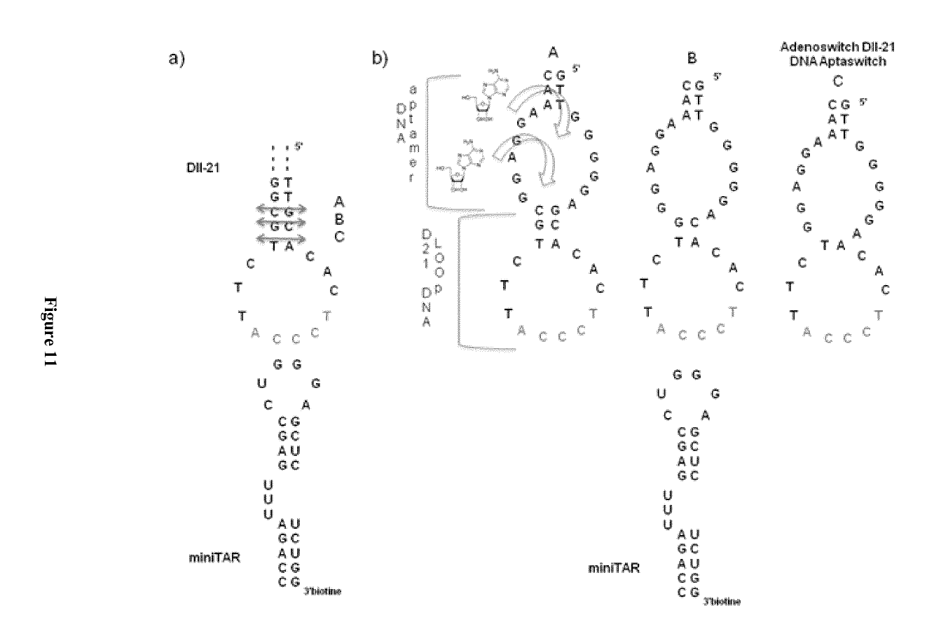

So we took advantage of this complex to replace the DNA apical loop of the aptamer directed against adenosine with the DNA loop of the DII21 aptamer able to form kissing complexes. Three models (A, B and C) having three different connectors introduced between the loop and the region of binding of adenosine have been synthesized (FIG. 11).

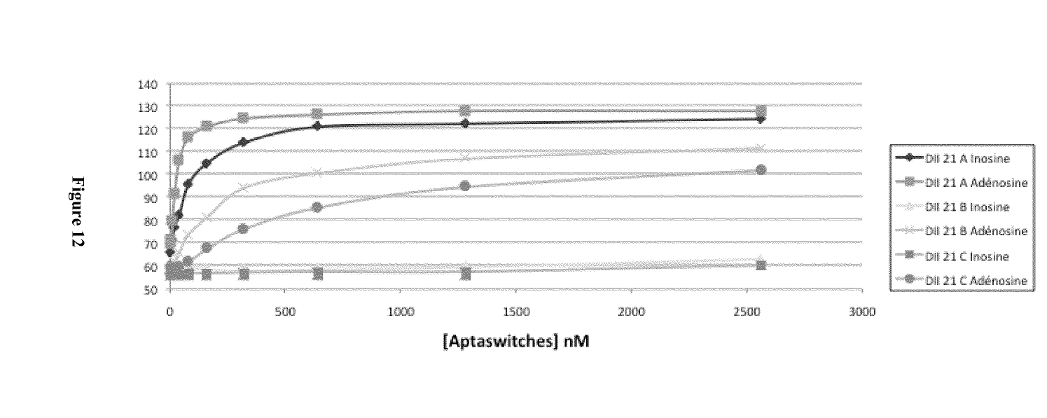

These three models were tested by fluorescence anisotropy for their ability to bind TAR labeled with a fluorescent group (Texas Red) in the presence on one hand of Inosine (control of specificity of adenosine) and on the other hand of adenosine (FIG. 12). The results show a strong non-specific binding of DII21 A in the presence of inosine. The aptaswitches DII21B and C show a highly specific binding to TAR.

These results demonstrate that fully DNA aptaswitches can be functional. Therefore our aptakiss-aptaswitch strategy is not restricted to RNA-RNA kissing complexes but can be extended to RNA-DNA kissing complexes. This possibility is not restricted to the DII21 sequence but can be extended to any sequence capable of generating RNA-DNA stable kissing complex.

### EXAMPLE 7

### DNA Combinatorial Libraries

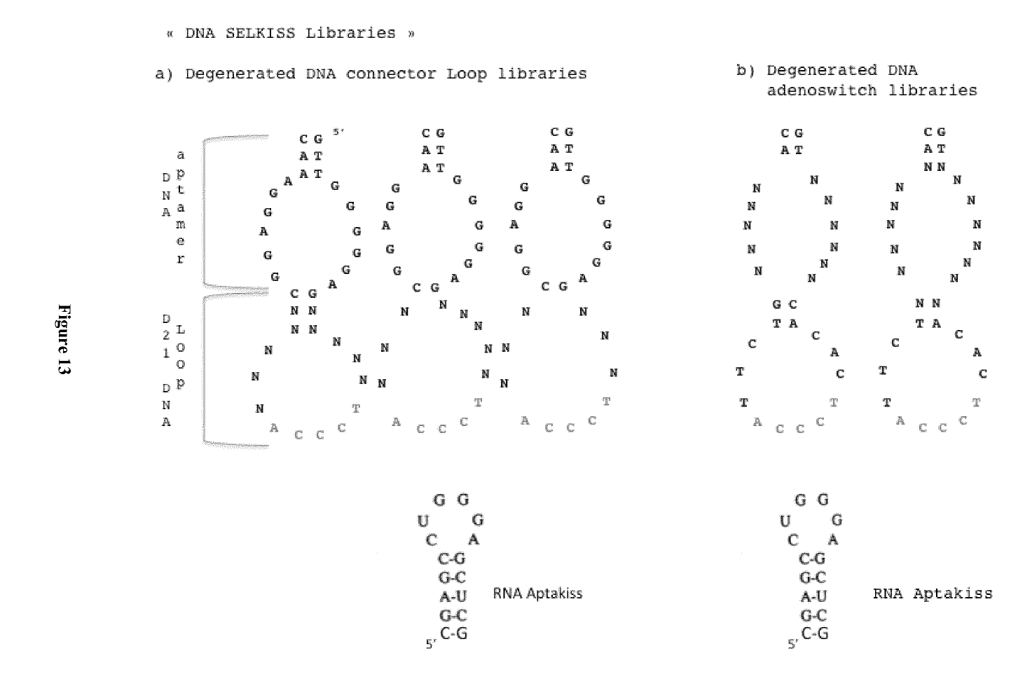

DNA libraries were synthesized based on the results obtained with the aptaswitch DII21B model to perform SELKISS (i.e. SELEX which implements kissing complex formation) with a DNA library. DNA libraries have been produced using the same desing as RNA libraries: one fixed region is flanked by two random windows which are themselves flanked by two primers. Five libraries with different random windows were designed with two distinct objectives: a) three libraries contain the random areas on the connector in order to find the best sequences that can bind TAR in the presence of adenosine, b) two libraries with the random windows in the region responsible for the binding to the adenosine (FIG. 13).

Initial analyzes by fluorescence anisotropy of the populations generated by DNA SELKISS show the existence of a small proportion of specific candidates for the adenosine. High throughput sequencing of samples obtained with different selection pressures (DNA and adenosine concentrations, dissociation time . . . ) allows to obtain aptaswitches families and to identify the specific sequences by checking their ability to bind TAR specifically.

The aptakiss used herein is always an RNA hairpin. However, experiments confirm that RNA 2′-fluoropyrimidine aptakiss could be used (data not shown).

Furthermore, in order to obtain DNA aptaswitches and DNA aptakiss, a SELEX of DNA kissing complexes was undertaken and is now being sequenced.

The DNA SELKISS provides DNA aptaswitches more resistant to nucleases than unmodified RNA. In addition, the cost of synthesis of DNA oligonucleotides is much lower than that of RNA (factor 5).

The present sensing format can be considered as an original sandwich-like assay for small ligand detection with unique binding specificity features originating from the double recognition mechanism involved in the ternary complex formation. Moreover, such sandwich-like assay could be easily adapted to the ELISA-type format to achieve signal amplification, by using both enzyme-linked aptakiss and surface-immobilized aptaswitch. Our strategy could also allow multiplexed analysis: we actually identified a repertoire of several kissing pairs that do not cross-interact. We can therefore introduce different kissing prone sequences in different aptamers thus generating a series of aptaswitches that could be used simultaneously as far as they could be monitored independently. As this approach can be adapted to any aptamer folded as imperfect hairpin whose apical loop is not crucial for the interaction with the ligand, aptakiss-aptaswitch combination has a wide potential interest for analytical applications.

SEQUENCE LISTING:

| SEQ ID | | |
|---|---|---|
| NO: | Name | Sequence |
| 1 | K10 | ACAGCTCAGAA |
| 2 | K11 | CTTCCGCAGGT |
| 3 | K13 | ACGCTGAGT |
| 4 | K15 | ATTGGTGGNC |
| 5 | K17 | TCCCCTGCA |
| 6 | K18 | ACGCGTCCT |
| 7 | K1 | ATGTGCGCCAA |
| 8 | K20 | GTGGGTAGGGT |
| 9 | K24 | CTAGCCGCGT |
| 10 | K25 | AATCTGTCCG |
| 11 | K27 | GTACGGTGGA |
| 12 | K28 | TTGCGCTTCT |
| 13 | K29 | GTAAGtCTGA |
| 14 | K30 | ATTGGgCCTG |
| 15 | K8 | GATAGGGGGGA |
| 16 | K2 | GATGACCGCG |
| 17 | K34 | ATAAGGACAT |
| 18 | K35 | GTAAAGCG |
| 19 | K14 | GTAACGCGCGGA |
| 20 | K16 | GTAACGCCCA |
| 21 | K3 | ATGGAT |
| 22 | K4 | TTTTGAGTGAT |
| 23 | K5 | ACTCCCACGCT |
| 24 | K6 | TGTAATTCGA |
| 25 | K9 | GGACAACGGT |
| 26 | K21 | TTTCCCCTCCG |
| 27 | K32 | TTGCCTCCGT |
| 28 | K37 | TGTCGCCTGT |
| 29 | K42 | TCTTCGCTATC |
| 30 | K38 | GCAGTTCACT |
| 31 | K36 | GCGTTCATTA |
| 32 | K39 | TCTTGGGGCA |
| 33 | K44 | TCTGCGGGACA |
| 34 | K40 | ACAAGTCTCG |
| 35 | K41 | TATGGACGGCT |
| 36 | K45 | TTGTGGTCGAG |
| 37 | K50 | aCGCCTTCAG |
| 38 | K51 | GATTTtTGGT |
| 39 | K52 | GAGGCGGAA |
| 40 | K53 | TCTGGCACAT |
| 41 | K55 | gAATgtTTtG |
| 42 | K56 | TCGTTTGTGT |
| 43 | K58 | AGGAGGTGGA |
| 44 | K59 | TGTGGCTTAG |
| 45 | K60 | CCcTCCTACTGT |
| 46 | K61 | TTGCGCCCTGT |
| 47 | K63 | GTGGGTAGTA |
| 48 | K64 | GAGAGTGGGG |
| 49 | K70 | GGTCCGAGGATC |
| 50 | K71 | TATTCTTCGG |
| 51 | K73 | AGCGATTGGA |
| 52 | K74 | TTTGAGGCCT |
| 53 | K75 | TCTTTGGTTT |
| 54 | K76 | ATTGTCCAcTG |
| 55 | K77 | AGACCGGCA |
| 56 | K78 | TTTGCGGCTA |
| 57 | K79 | GTTTTGTGTA |
| 58 | K80 | ATGCCGTGAT |
| 59 | K82 | GAGtGgAAGG |
| 60 | K85 | GTCACA |
| 61 | K86 | CTTCCTGTTT |
| 62 | K87 | TTTGCGGCTA |
| 63 | K88 | GATAGTTTAgA |
| 64 | K89 | GCAATTATGA |
| 65 | K90 | TAATGGTGGGT |
| 66 | K91 | ATTGTACGGG |
| 67 | K92 | GTCGGTCA |
| 68 | K94 | GCTCTTGCTG |
| 69 | K95 | CTTTCCTCTCT |
| 70 | K96 | ACAGTGCAGC |
| 71 | K97 | GGGTGACGTT |
| 72 | K98 | ACGCGTACCT |
| 73 | K99 | ATCGTGGTAG |
| 74 | K101 | GGGTAGGTTGC |
| 75 | K102 | TATACGCGCAT |
| 76 | K103 | CGTGAGGGAT |
| 77 | K104 | ATTtAGCATT |
| 78 | K105 | TCGATGGNNNT |
| 79 | K107 | TATGGCACGAA |
| 80 | K108 | GCGTCCATGT |
| 81 | K109 | GTTGGCCGGG |
| 82 | K110 | TCGTAGGCTT |
| 83 | K111 | GACCGGCCCCT |
| 84 | K112 | GCGCCGTTAC |
| 85 | K113 | GTGCTGACAT |
| 86 | K115 | TCCCCGATcG |
| 87 | K116 | GGGTAGAGAA |
| 88 | K117 | ATTTGTACGT |
| 89 | K118 | GACGTCTATGG |
| 90 | K119 | AGTGGCTGGG |
| 91 | K120 | GATGGCGGCT |
| 92 | K121 | TGCACATATT |
| 93 | K122 | GTTATTGTTC |
| 94 | K123 | GGTACCGTTA |
| 95 | K124 | TATCCCTTTG |
| 96 | K126 | ATAGAGCCCTT |
| 97 | K128 | TTGGTTTTGT |
| 98 | K129 | CCGTGAGCAA |
| 99 | K131 | GGTTGTAGTT |
| 100 | K132 | TCCGTCCGAG |
| 101 | K133 | CGTCCTGCGa |
| 102 | K134 | GTATGGGTGT |
| 103 | K137 | TGGTATTGTG |
| 104 | K138 | GGTCCAAAGT |
| 105 | K139 | GTATTGGGCA |
| 106 | K141 | CCTGGaCCTT |
| 107 | K142 | AGATCAGCGCG |
| 108 | K143 | ATTAGCCTGG |
| 109 | K144 | GGTTATACT |
| 110 | K145 | TTTGCGGCGGA |
| 111 | | GCCCCG |
| 112 | | GCCUCG |
| 113 | | GCCGCG |
| 114 | | GCCUUG |
| 115 | | ACCGCG |
| 116 | | ACCACG |
| 117 | | ACCCCG |
| 118 | | ACCUUG |
| 119 | | GCCCCU |
| 120 | | GCCGCU |
| 121 | | GCCUCU |
| 122 | | UCCCUG |
| 123 | | UCCACG |
| 124 | | UCCAUG |
| 125 | | UCCUUG |
| 126 | | ACCGCC |
| 127 | | UCCGCC |
| 128 | | CCCGCU |
| 129 | | CCCAUC |
| 130 | | GCCAUC |
| 131 | | UCCAUC |
| 132 | | ACCAUU |
| 133 | | CCCAUU |
| 134 | | UCCACU |
| 135 | | GCCCCA |
| 136 | | GCCCCC |
| 137 | | ACCUCA |
| 138 | | CCCUCC |
| 139 | | CCCUCG |
| 140 | | CCCUUC |
| 141 | | ACCCCC |
| 142 | | UCCCCC |
| 143 | | CCCCCC |
| 144 | | CCCCCU |
| 145 | | ACCUCU |
| 146 | | CCCCUC |
| 147 | | UGGGGC |
| 148 | | GGGGGG |
| 149 | | AGGGGA |
| 150 | | GGAGGG |
| 151 | | GGAGGU |
| 152 | | UGCGGC |
| 153 | | UGCGGU |
| 154 | | UGCGGG |
| 155 | | AGCGGG |
| 156 | | GAAGGU |
| 157 | | AGCGGG |
| 158 | | GGCGGG |
| 159 | | UGCGGG |
| 160 | | GGUGGU |
| 161 | | GGUGGG |
| 162 | | UGGGGU |
| 163 | | AGGGGG |
| 164 | | GGGGGC |
| 165 | | GGGGGG |
| 166 | | CAAGGG |
| 167 | | GGGGGU |
| 168 | | CGGGGC |
| 169 | | GGCGGU |
| 170 | | CGCGGU |
| 171 | | CGCGGA |
| 172 | | UGAGGC |
| 173 | | AGAGGG |
| 174 | | GAGGGG |
| 175 | | GAGGGA |
| 176 | | UAGGGA |
| 177 | | UGUGGC |
| 178 | | GGUGGU |
| 179 | | AGUGGC |
| 180 | | CGUGGU |
| 181 | | GGUGGG |
| 182 | | AGUGGG |
| 183 | | AAUGGA |
| 184 | | GAAGGG |
| 185 | | GGCGGU |
| 186 | | GGCGGG |
| 187 | | GGCGGA |
| 188 | | UAUGGC |
| 189 | | GGUGGG |
| 190 | | AGUGGA |
| 191 | | GGGGGU |
| 192 | | AGGGGA |
| 193 | | GGAGGG |
| 194 | | UGAGGA |
| 195 | | UGAGGG |
| 196 | | GAAGGC |
| 197 | | AAAGGA |
| 198 | | GGGGGG |
| 199 | | AGGGGG |
| 200 | | GGGGGU |
| 201 | | UGGGGA |
| 202 | | UGAGGA |
| 203 | | UGAGGG |
| 204 | | UAGGGA |
| 205 | NS1 | UGCUCG |
| 206 | NS2 | CGAGCA |
| 207 | NS3 | ACGAGC |
| 208 | NS4 | GCUCGU |
| 209 | KG51 | ACGAGCUGGGGCGCUCGU |
| 210 | KC24 [Aptakiss] | UGCUCGGCCCCGCGAGCA |
| 211 | Adenoswitch | TGGGGGACUGGGGCGGGAGGAA |
| 212 | AdenoswitchTA | TTGGGGGACUGGGGCGGGAGGAAA |
| 213 | AdenoswitchTAGC | GTTGGGGGACUGGGGCGGGAGGAAAC |
| 214 | | UGCUCGGCCCCGCGAGCA |
| 215 | | CUGGGGCG |
| 216 | | UGCUCGGCCCCGCGAGCA |
| 217 | | GGUUACCAGCCUUCACUGCUCG-N10/11- |
| | | CGAGCACCACGGUCGGUCACAC |
| 218 | | ACGAGC-NRNGGN-GCUCGU |
| 219 | | GGUUACCAGCCUUCACUGCUCG-NCCNYN- |
| | | CGAGCACCACGGUCGGUCACAC |
| 220 | | GGGAGGACGAAGCGGACGAGC-NRNGGN- |
| | | GCUCGUCAGAAGACACGCCCGA |
| 221 | Primer | TAATACGACTCACTATAGGTTACCAGCCTTCACTGC |
| 222 | Primer | TAATACGACTCACTATAGGGAGGACGAAGCGG |
| 223 | Primer | TCGGGCGTGTCTTCTG |
| 224 | K18 (long) | UGCUCGACGCGUCCUCGGCA |
| 225 | Kx3 | GGUCGGUCCCAGACGACC |
| 226 | Kx4 | GGUUUCAGGGCAGUGAUGUUGCCCCUCGGAAGAUAACC |
| 227 | Kx3′ | CGAGCCUGGGAGCUCG |
| 228 | Kx4′ | CCUGACAUCACCAGG |
| 229 | GTPswitch | UCCGAAGUGGUUGGGCUGGGGCGUGUGAAAACGGA |
| 230 | Aptakissmut | UGCUCGGCCGCGCGAGCA |
| 231 | adenoswitchmut | TGGGGGACUGCGGCGGGAGGAA |

### REFERENCES

Throughout this application, various references describe the state of the art to which this invention pertains. The disclosures of these references are hereby incorporated by reference into the present disclosure.

1. A kit-of-parts comprising at least one nucleic acid molecule NA1 and at least one nucleic acid molecule NA2 wherein:
a) the nucleic acid molecule NA1 comprises a nucleotide acid sequence NS1-NSK1-NS2, wherein
NS1 and NS2 are at least 1 nucleotide in length, and NS1 and NS2 are complementary sequences;
NSK1 has a nucleotide acid sequence of at least 2 nucleotides,
b) the nucleic acid molecule NA2 comprises a nucleotide sequence NS3-NSK2-NS4 wherein:
NS3 and NS4 are at least 1 nucleotide in length, and NS3 and NS4 are complementary sequences;
NSK2 has a nucleotide acid sequence of at least 2 nucleotides
c) NA1 and NA2 each form under appropriate conditions at least one hairpin loop comprising NSK1 and NSK2 respectively; and
d) NA1 and NA2 form a duplex by an interaction of NSK1 and NSK2 which forms a kissing complex between hairpin loops comprising NSK1 and NSK2; and
e) at least one of NA1 or NA2 is an aptamer exhibiting specificity and affinity for a target molecule.

2. (canceled)

3. The kit-of-parts of claim 1 wherein one or both of NKS1 and NSK2 has a nucleotide acid sequence of 2, 3, 4, 5, 6, 7, 8, 9, 10, 11, 12, 13, 14, or 15 nucleotides.

4. (canceled)

5. The kit-of-parts of claim 1 which comprises a first nucleic acid molecule NA1 wherein NSK1 has a sequence motif selected from the group consisting of YRYR, RYRY, YYRY, RYRR, YYYR, YRYY, RYYR, YRRY, YRRR, RYYY, RRYR, RRYY, RRRR, RRRY, YYYY, YYRR.

6. The kit-of-parts of claim 1 wherein NSK1 is represented by Kn and NKS2 is represented by Kn′, wherein Kn and Kn′ are selected from Table B or Table C1, and Kn and Kn′ may be identical or not.

7. (canceled)

8. The kit-of-parts of claim 1 wherein NSK1 comprises a nucleic acid sequence motif CCNY and NKS2 comprises a nucleic acid sequence motif RNGG.

9. The kit-of-parts of claim 1 wherein NSK1 comprises a nucleic acid sequence motif NCCNYN and comprises a nucleic acid sequence motif NRNGGN.

10. The kit-of-parts of claim 1 wherein NSK1 comprises a nucleic acid sequence motif NCCNYN and wherein NKS2 comprises a nucleic acid sequence motif NRNGGN, wherein sequence motif NCCNYN and sequence motif NRNGGN are selected from Table C2.

11. The kit-of-parts according to claim 1 wherein each of NS1, NS2, NS3 and NS4 comprise 1, 2, 3, 4, 5, 6, 7, 8, 9, 10, 11, 12, 13, 14 or 15 nucleotides.

12. The kit-of-parts according to claim 1 wherein NS1 has a nucleotide sequence UGCUCG and NS2 has a nucleotide sequence CGAGCA.

13. The kit-of-parts according to claim 1 wherein NS3 has a nucleotide sequence ACGAGC and NS4 has a nucleotide sequence GCUCGU.

14. The kit-of-parts of claim 1 wherein NA1 is or comprises a nucleic acid sequence selected form the group consisting of: ACGAGCUGGGGCGCUCGU (KG51); TGGGGGACUGGGGCGGGAGGAA; GTTGGGGGACUGGGGCGGGAGGAAAC; and UCCGAAGUGGUUGGGCUGGGGCGUGUGAAAACGGA; and NA2 comprises a nucleic acid sequence UGCUCGGCCCCGCGAGCA (KC24-Aptakiss).

15-18. (canceled)

19. The kit-of-parts of claim 1, wherein the aptamer is specific for a small organic molecule, in particular a small organic molecule which contains at least one aromatic ring group.

20. The kit-of-parts of claim 1, wherein the aptamer forms a duplex only when it binds to the target molecule.

21. (canceled)

22. The kit-of-parts of claim 1 wherein at least one of NA1 and NA2 is labeled with a detectable label selected from the group consisting of a visual label, an optical label, a photonic label, an electronic label, an acoustic label, an opto-acoustic label, a mass label, an electro-chemical label, an electro-optical label, a spectrometry label, and an enzymatic label.

23. The kit-of-parts of claim 1 wherein at least one of NA1 or NA2 is immobilized on a solid support.

24. A combinatorial random library comprising nucleic acid molecules each of which has an internal region comprising a sequence NA1 or NA2 that forms a kissing complex which is flanked by at least one variable region.

25. The combinatorial random library of claim 24 comprising a plurality of nucleic acid molecules having the general formula
5′-P1-V-NSKn-P2-3′ or 5′-P1-NSKn-V-P2-3′ wherein P1 and P2 represent primer regions, V represents a variable region of at least 2 nucleotides, and NSKn represents a nucleic acid sequence NSK1 or a nucleic acid sequence NSK2, or
5′-P1-V1-NSKn-V2-P2-3′ wherein P1 and P2 represent primer regions, V1 and V2 represent variable regions of at least 2 nucleotides, and NSKn represents a nucleic acid sequence NSK1 or a nucleic acid sequence NSK2, or
5′-P1-Xn-V1-NSKn-V2-Yn-P2 wherein P1 and P2 represent primer regions, V1 and V2 represent variable regions of at least 2 nucleotides, Xn and Yn represent nucleotide sequences of 1, 2, 3 or more nucleotides and Xn and Yn can hybridize, and NSKn represents a nucleic acid sequence NSK1 or a nucleic acid sequence NSK2;
wherein
NSKn has a nucleotide acid sequence of at least 2 nucleotides, the nucleic acid molecules form, under appropriate conditions, at least one hairpin loop comprising NSKn; and
NSKn is able to form a kissing complex with another hairpin loop.

26. The combinatorial random library of claim 24 wherein the variable region comprises 2; 3; 4; 5; 6; 7; 8; 9; 10; 11; 12; 13; 14; 15; 16; 17; 18; 19; 20; 21; 22; 23; 24; 25; 26; 27; 28; 29; 30 or more nucleotides.

27. (canceled)

28. The combinatorial random library of claim 24, wherein NSKn has a sequence motif selected from the group consisting of YRYR, RYRY, YYRY, RYRR, YYYR, YRYY, RYYR, YRRY, YRRR, RYYY, RRYR, RRYY, RRRR, RRRY, YYYY, and YYRR.

29. The combinatorial random library of claim 24, wherein NSK1 and NSK2 are represented by Kn and Kn′, wherein Kn and Kn′ are selected from Table B or Table C1.

30. The combinatorial random library of claim 24, wherein NSK1 comprises a nucleic acid sequence motif CCNY and NKS2 comprises a nucleic acid sequence motif RNGG.

31. The combinatorial random library of claim 24, wherein NSK1 comprises a nucleic acid sequence motif NCCNYN and NKS2 comprises a nucleic acid sequence motif NRNGGN.

32. The combinatorial random library of claim 31, wherein sequence motif NCCNYN and sequence motif NRNGGN are selected from Table C2.

33. The combinatorial random library of claim 24, wherein the variable regions V1 and V2 comprise 5; 6; 7; 8; 9; 10; 11; 12; 13; 14; 15; 16; 17; 18; 19; 20; 21; 22; 23; 24; 25; 26; 27; 28; 29; or 30 nucleotides.

34. The combinatorial random library of claim 24, wherein one or both of Xn and Yn represents a nucleotide sequence of 1; 2; 3; 4; 5; 6; 7; 8; 9; 10; 11; 12; 13; 14; 15; 16; 17; 18; 19; 20; 21; 22; 23; 24; 25; 26; 27; 28; 29; or 30 nucleotides.

35. (canceled)

36. A method for identifying an aptamer comprising a nucleic acid sequence having affinity for a target molecule, comprising:
i) contacting the target molecule with a combinatorial random library according to claim 24, thereby forming a mixture comprising the target molecule, a nucleic acid molecule comprising NSK1, and a nucleic acid molecule comprising NSK2;
iii) partitioning nucleic acids having affinity for the target molecule from nucleic acids not having affinity for the target molecule by detecting duplexes formed between the nucleic acid molecule comprising NSK1 and the nucleic acid molecule comprising NSK2, wherein detection of duplexes indicates that a nucleic acid sequence having affinity for the target molecule is present in the aptamer.

37-44. (canceled)

45. A method for detecting at least one target molecule in a sample comprising
i) providing a kit-of-parts according to claim 1 which comprises nucleic acid molecules NA1 and NA2, at least one of which is an aptamer specific for the at least one target molecule, wherein NA1 and NA2 form a complex only when the aptamer binds to the at least one target molecule;
ii) contacting the sample with the nucleic acid molecules NA1 and NA2 and
iii) detecting duplexes formed between the nucleic acid molecules NA1 and NA2, wherein detection of duplexes indicates the presence of the at least one target molecule in the sample.

46. The method of claim 45, wherein step i) comprises providing a plurality of kit-of-parts each of which comprises an aptamer specific for a different target molecule, thereby detecting the presence or the absence of a plurality of different target molecules in the sample.

47. The method of claim 45 wherein the target molecule is a small organic molecule.

48. The method of claim 45, wherein the sample is selected from the group consisting of biological material that have been isolated from individuals, such as, biological tissues and fluids, which include blood, skin, plasma, serum, lymph, urine, cerebrospinal fluid, tears, smears, a sample of water, in particular drinking water, ground water, surface water or wastewater sample, a sample prepared from a material from the environment, a clinical specimen and a food sample.

49. (canceled)

50. The method of claim 45, wherein detection is conducted in a liquid phase by chromatography, electrophoresis, or filtration.

51-56. (canceled)

57. A nucleic acid molecule which is or which includes a sequence selected from the group consisting of ACGAGCUGGGGCGCUCGU, UGCUCGGCCCCGCGAGCA, TGGGGGACUGGGGCGGGAGGAA, TTGGGGGACUGGGGCGGGAGGAAA, GTTGGGGGACUGGGGCGGGAGGAAAC, UCCGAAGUGGUUGGGCUGGGGCGUGUGAAAACGGA, UGCUCGGCCGCGCGAGCA, and TGGGGGACUGCGGCGGGAGGAA.
